(19) United States
(12) Patent Application Publication
(10) Pub. No.: US 2019/0262826 A1
(43) Pub. Date: Aug. 29, 2019

(54) Reaction Vessel Holder And Molecule Detection Device

(71) Applicant: Ubiquitome Limited, Auckland (NZ)

(72) Inventor: Christopher Bruce Rawle, Dunedin (NZ)

(21) Appl. No.: 16/406,423

(22) Filed: May 8, 2019

Related U.S. Application Data
(63) Continuation of application No. 14/773,077, filed on Sep. 4, 2015, now abandoned.
(60) Provisional application No. 61/875,229, filed on Sep. 9, 2013.
(60) Provisional application No. 61/774,814, filed on Mar. 8, 2013.

(51) Int. Cl. B01L 3/00; G01N 21/64; G01N 21/03; B01L 7/00; C12Q 1/68

(52) U.S. Cl. CPC B01L 3/50851; G01N 21/6452; G01N 21/0332; B01L 7/52; G01N 21/6428; H01L 35/30; G01N 2021/0371; B01L 2300/1822; B01L 2200/025; B01L 2300/168; B01L 2300/1894; C12Q 1/68

(57) ABSTRACT

A reaction vessel holder (120) receives a reaction vessel (110). The reaction vessel (110) has a portion that is substantially optically transparent to light of a first range of wavelengths. The reaction vessel holder (120) comprises a body having a high thermal conductivity that is thermally coupled to and supports the reaction vessel (120). The body is further thermally coupled to a thermal device (130) for heating or cooling the reaction vessel holder (120) and thereby the reaction vessel (110). The body comprises a transparent portion that is substantially optically transparent to the light of the first range of wavelengths. The optically transparent portion of the reaction vessel (110) faces the transparent portion of the body such that light of the first range of wavelengths to and/or from the sample in the reaction vessel (120) can pass through the transparent portion.

### FIELD OF THE INVENTION

The present invention generally relates to a reaction vessel holder and to a molecule detection device comprising the reaction vessel holder.

### BACKGROUND

Systems and methods for molecule detection are widely known and used.

Amplification of nucleic acids can be performed using quantitative polymerase chain reaction (Q-PCR) analysis for example. Generally for a Q-PCR analysis, the temperature of a sample within a reaction vessel is repeatedly cycled between a higher temperature at which the template DNA is denatured, and a lower temperature at which the primers in the sample anneal to a targeted DNA sequence and the DNA replicates. This thermal cycling is commonly repeated up to 40 times until the DNA in the sample is amplified or replicated sufficiently to enable detection of a fluorescing reagent dye bound to the DNA.

Proteins within a sample can be detected through antibody binding approaches for example. Fluorescently labelled antibodies can be mixed with a sample and the protein-antibody complexes captured. Presence of florescence indicates the presence of the protein in the sample.

Detection of metabolites can be performed for example. For this use a sample is mixed with a reporter dye. The metabolite in the sample either directly or indirectly converts the reporter to a fluorescent dye in proportion to the amount of metabolite in the original sample.

The Q-PCR method is described generally in U.S. Pat. No. 5,994,056 to Russell Higuchi entitled ‘Homogenous methods for nucleic acid amplification and detection’, for example. This document discloses a method for detecting amplification by exposing the reaction mixture to ultraviolet light and detecting fluorescence of ethidium bromide fluorescent dye using a spectra fluorometer.

Various apparatuses for performing Q-PCR analysis, protein analysis, or ligand analysis are commercially available and used in laboratories by skilled and trained users to amplify and quantify a targeted molecule.

Existing apparatuses for detection of molecules are generally designed exclusively for use in a laboratory environment, and may be adapted to thermally cycle a large array of samples (for example, in 96 or more reaction vessels or wells as in a microtiter plate) at the same time. As a result, these apparatuses are generally relatively large, heavy, and inefficient in terms of power use, in particular when analysing only a small number of samples. These apparatuses are also commonly expensive and/or complex.

An example of such an apparatus is described in U.S. Pat. No. 6,814,934 to Russell Higuchi, entitled ‘Instrument for monitoring nucleic acid amplification’. This document discloses a detection system comprising a thermal cycler and an independently-housed spectra fluorometer, whereby the fluorometer is optically coupled with the reaction vessels by way of fibre optic cables. Although integration of the thermal cycler and fluorometer is suggested in this document, there is no detailed disclosure of an integrated apparatus, let alone an apparatus which is small, efficient, and portable.

There is a need for a suitable apparatus for detecting molecule(s) which is compact and portable, robust, efficient and/or relatively simple to operate. Such a portable apparatus has potential uses ‘in the field’ (ie outside the laboratory environment) by semi-skilled users with limited or no training, for environmental testing for example.

Some systems have detector arrangement and excitation arrangement positioned facing opposite sides of a reaction vessel. However, the applications of these devices are limited. The reaction vessel cannot be heated by the system, and needs to be heated as a separate step prior to being analysed by the system.

It is an object of at least preferred embodiments of the present invention to provide a reaction vessel holder that allows for a compact molecule detection device that addresses one or more of the disadvantages of the existing devices, or to at least provide the public with a useful alternative.

### SUMMARY OF THE INVENTION

In a first aspect, the present invention provides a reaction vessel holder for receiving a reaction vessel, the reaction vessel for containing a sample and having at least one portion that is substantially optically transparent to light of at least a first range of wavelengths, the reaction vessel holder comprising:
a body having a high thermal conductivity, the body being arranged to be thermally coupled to and support the reaction vessel, the body further being arranged to be thermally coupled to a thermal device for heating or cooling the reaction vessel holder and thereby the reaction vessel, the body comprising at least one transparent portion that is substantially optically transparent to the light of at least the first range of wavelengths, such that the optically transparent portion of the reaction vessel is adapted to face the transparent portion of the body such that light of the first range of wavelengths to and/or from the sample in the reaction vessel can pass through the transparent portion.

In an embodiment, the thermal conductivity of the body is about 25 Wm⁻¹K⁻¹ or higher. In a further embodiment, the thermal conductivity of the body is about 285 Wm⁻¹K⁻¹ or higher. In a further embodiment, the thermal conductivity of the body is higher than about 1500 Wm⁻¹K⁻¹. In a further embodiment, the thermal conductivity of the body is between about 1800 Wm⁻¹K⁻¹ and about 2100 Wm⁻¹K⁻¹.

In an embodiment, the body comprises a low thermal mass. In a further embodiment, the body comprises a low specific heat capacity. In a further embodiment, the specific heat capacity of the body is less than about 1.0 Jg⁻¹K⁻¹ at about 300K. In a further embodiment, the specific heat capacity of the body is less than about 0.8 Jg⁻¹K⁻¹ at about 300K. In a further embodiment, the specific heat capacity of the body is less than about 0.6 Jg⁻¹K⁻¹ at about 300K. In a further embodiment, the specific heat capacity of the body is about 0.418 Jg⁻¹K⁻¹.

In an embodiment, the mass of the body is between about 1 g and about 10 g. In an embodiment, the mass of the body is between about 1 g and about 5 g. In an embodiment, the mass of the body is between about 1 g and about 2.5 g. In an embodiment, the mass of the body is between about 1 g and about 2 g. In an embodiment, the mass of the body is about 1.9 g.

In an embodiment, the body has a low thermal expansion coefficient. In a further embodiment, the thermal expansion coefficient of the body is less that about 1×10⁻⁵ K⁻¹ at about 300K. In a further embodiment, the thermal expansion coefficient of the body is less than about 6.0×10⁻⁶ K⁻¹ at about 300K. In a further embodiment, the thermal expansion coefficient of the body is less that about 5.5×10⁻⁶ K⁻¹ at about 300K. In a further embodiment, the thermal expansion coefficient of the body is between about 0.8×10⁻⁶ K⁻¹ and about 1.2×10⁻⁶ K⁻¹ at about 300K. In a further embodiment, the thermal expansion of the body is about 1.0×10⁻⁶ K⁻¹ at about 300K.

In an embodiment, the body is an optically transparent plate. In a further embodiment, the plate is substantially formed of a synthetic diamond material. In a further embodiment, the synthetic diamond material is produced by chemical vapour deposition. In an alternative embodiment, the synthetic diamond material is produced by a high-pressure high temperature formation method. In an additional or alternative embodiment, the plate is formed of synthetic sapphire (Corundum or aluminium oxide Al₂O₃). In an additional or alternative embodiment, the plate is formed of substantially optically transparent aluminium nitride (AlN).

In an embodiment, where the thermal device is a single thermoelectric cooling unit, the body is configured to be thermally cycled at up to about 20° C. per second. In a further embodiment, where the thermal device comprises two thermoelectric cooling units, the body is configured to be thermally cycled at up to about 40° C. per second.

In an embodiment, the body is arranged to be physically mounted to the thermal device. In a further embodiment, the thermal device comprises a thermoelectric cooling unit. In a further embodiment, the thermal device comprises a plurality of thermoelectric cooling units. In a further embodiment, the thermal device comprises two thermoelectric cooling units, and the body is physically mounted to each of the thermoelectric cooling units. In a further embodiment, the reaction vessel holder comprises a thermal coupling medium for thermally coupling the body and the thermoelectric cooling unit. In a further embodiment, the thermal coupling medium comprises a heat sink paste. In a further embodiment, the thermal coupling medium comprises a silver compound heat sink paste and/or indium foil. In a further embodiment, an end of the body is mounted to one of the thermoelectric cooling units and an opposite end of the body is mounted to the other thermoelectric cooling unit.

In an embodiment, the reaction vessel holder comprises a member for securing the body to the thermal device. In a further embodiment, the member comprises a clamp or a fastener. In a further embodiment, the clamp comprises a plastic material. In a further embodiment, the clamp has a low thermal conductivity of about 0.2 Wm⁻¹K⁻¹. In a further embodiment, where the thermal device comprises two thermoelectric cooling units, the device comprises two clamps, wherein each clamp is adapted to secure the body to a respective one of the thermoelectric cooling units. In a further embodiment, each clamp is positioned above a respective one of the thermoelectric cooling units. In a further embodiment, each clamp is centrally positioned above a respective one of the thermoelectric cooling units.

In an embodiment, the reaction vessel holder further comprises a temperature sensor for sensing the temperature of the body. The temperature sensor may be separate from or integral with the body. In a further embodiment, the temperature sensor is in electronic communication with a controller for controlling an operation of the thermal device for heating and/or cooling the body.

In an embodiment, the reaction vessel holder comprises a thermal coupling medium for thermally coupling the reaction vessel to the body. In a further embodiment, the thermal coupling medium comprises a thermal coupling oil.

In an embodiment, the reaction vessel holder comprises a reaction vessel securing member for removably securing the reaction vessel to the body. In a further embodiment, the reaction vessel securing member is configured to apply a downward force onto the reaction vessel when the reaction vessel is placed on top of the body for increasing the physical and thermal contact between the reaction vessel and the body.

In an alternative embodiment, the reaction vessel holder comprises two reaction vessel securing members for securing opposite sides or ends of the reaction vessel. In one embodiment, the reaction vessel securing member(s) is/are moveable between a release configuration in which the reaction vessel can be positioned on or removed from the body and a secure configuration in which the reaction vessel is secured to the body.

In an embodiment, the reaction vessel holder comprises a filter for passing light having the first range of wavelengths and for blocking or reflecting light with at least a second range of wavelengths. In a further embodiment, the filter is separate from or integral with the body. In a further embodiment, the filter is an optical coating on a surface of the transparent portion of the body.

In an embodiment, the first range of wavelengths comprises a range of excitation wavelengths of an excitation beam for exciting an emission of reaction light from the sample, and the second range of wavelengths comprises a range of reaction light wavelengths of reaction light from the sample. In an alternative embodiment, the first range of wavelengths comprises a range of reaction light wavelengths of reaction light from the sample, and the second range of wavelengths comprise excitation wavelengths of an excitation beam for exciting an emission of reaction light from the sample.

In an embodiment, the range of excitation wavelengths and the range of reaction light wavelengths are dependent on a dye used in the sample, and comprise the following excitation wavelength and reaction light wavelength when the following dyes are used (dye—excitation wavelength in nm—reaction light wavelength in nm): SYBR—497-520; FAM—495-520; TET—521-536; JOE—520-548; VIC—538-554; HEX—535-556; R6G—524-557; Cy3—550-570; TAMRA—555-576; NED—546-575; Cy3.5—581-596; ROX—575-602; Texas Red—583-603; Cy5—649-670; Cy5.5—675-694.

In a further embodiment, said at least one transparent portion of the body is substantially optically transparent to light having wavelengths between about 400 nm and about 800 nm. In a further embodiment, said at least one transparent portion of the body is substantially optically transparent to light having wavelengths greater than about 225 nm.

According to a second aspect, the present invention provides a device for molecule analysis of a sample in a reaction vessel, the reaction vessel having at least one portion that is substantially optically transparent to a light of at least a first range of wavelengths, the device comprising the reaction vessel holder of the first aspect described above.

The device of the second aspect of the invention may have one or more features outlined in relation to the first aspect above.

In an embodiment, the device comprises a thermal device that is thermally coupled to the reaction vessel holder. In a further embodiment, the thermal device comprises a thermoelectric cooling unit. In a further embodiment, the thermal device comprises a plurality of thermoelectric cooling units. In a further embodiment, the thermal device comprises two thermoelectric cooling units. In a further embodiment, where the thermal device is a single thermoelectric cooling unit, the body is configured to be thermally cycled at up to about 20° C. per second. In a further embodiment, where the thermal device comprises two thermoelectric cooling units, the body is configured to be thermally cycled at up to about 40° C. per second. In a further embodiment, the thermal device comprises a heat source thermally coupled to the reaction vessel holder, which is activated when a temperature of the device is below a temperature threshold. In a further embodiment, the heat source comprises a resistive heater.

In an embodiment, the reaction vessel holder is arranged to be physically mounted to the thermal device. In a further embodiment, where the thermal device comprises two thermoelectric cooling units, the reaction vessel holder is physically mounted to each of the thermoelectric cooling units. In a further embodiment, an end of the reaction vessel holder is mounted to one of the thermoelectric cooling unit and an opposite end of the reaction vessel holder is mounted to the other thermoelectric cooling unit.

In an embodiment, the device comprises a member for securing the reaction vessel holder to the thermal device. In a further embodiment, the member comprises a clamp or a fastener. In a further embodiment, the fastener has a low thermal conductivity. In a further embodiment, where the thermal device comprises two thermoelectric cooling units, the device comprises two clamps, wherein each clamp is adapted to secure the reaction vessel holder to a respective one of the thermoelectric cooling unit.

In an embodiment, the device further comprises a temperature sensor for sensing the temperature of the reaction vessel holder. In a further embodiment, the temperature sensor is separate from or integral with the reaction vessel holder. In a further embodiment, the temperature sensor is in electronic communication with a controller for controlling an operation of the thermal device for heating and/or cooling the reaction vessel holder. In a further embodiment, the temperature sensor comprises a resistive temperature detector. In a further embodiment, the resistive temperature detector comprises a 100 ohm platinum resistive temperature detector.

In an embodiment, the device comprises a thermal coupling medium for thermally coupling the reaction vessel to the reaction vessel holder. In a further embodiment, the thermal coupling medium comprises a thermal coupling oil.

In an embodiment, the device comprises a reaction vessel securing member for removably securing the reaction vessel to the reaction vessel holder. In a further embodiment, the reaction vessel securing member is configured to apply a downward force onto the reaction vessel when the reaction vessel is placed on top of the reaction vessel holder for increasing the physical and thermal contact between the reaction vessel and the reaction vessel holder. In an alternative embodiment, the device comprises two reaction vessel securing members for securing opposite sides or ends of the reaction vessel. In one embodiment, the reaction vessel securing member(s) is/are moveable between a release configuration in which the reaction vessel can be positioned on or removed from the reaction vessel holder and a secure configuration in which the reaction vessel is secured to the reaction vessel holder.

In an embodiment, the device comprises a filter for passing light having the first range of wavelengths and for blocking or reflecting beams with at least a second range of wavelengths.

In an embodiment, the device comprises an excitation arrangement for generating one or more excitation beams for stimulating an emission of reaction light from the sample. In a further embodiment, the device comprises a detector arrangement for detecting the reaction light from the sample. In a further embodiment, the reaction vessel comprises a further portion that is substantially optically transparent to a light of at least a second range of wavelengths, and the excitation arrangement and the detector arrangement are positioned on or facing different sides of the reaction vessel. In a further embodiment, the excitation arrangement and the detector arrangement are positioned on or facing opposite sides of the reaction vessel. In a further embodiment, the reaction vessel holder is positioned between the excitation arrangement and the reaction vessel. In an alternative embodiment, the reaction vessel holder may be positioned between the detector arrangement and the reaction vessel.

In an embodiment, the device comprises a collimator. In a further embodiment, the collimator comprises a collimating lens. In a further embodiment, the collimator is integral with or separate from the excitation sources.

In an embodiment, the device further comprises an attenuator for reducing the power of the excitation beam. In a further embodiment, the attenuator comprises a neutral density (ND) filter. In a further embodiment, the attenuator reduces the power of the excitation beam by a factor of about 10. In a further embodiment, the attenuator is positioned between the collimator and the beam splitter arrangement. In a further embodiment, about 100 mW is incident on the ND filter, and about 10 mW exits the ND filter to the beam splitter arrangement. In an alternative embodiment, the optical assembly may comprise at least one attenuator for reducing the power of at least one of the plurality of split excitation beams from the beam splitter arrangement.

In an embodiment, the device further comprises a wavelength filter for filtering any spectral components in the excitation beam that fall within a band of the reaction light from the sample in at least one of the reaction vessels. In a further embodiment, the wavelength filter comprises a laser diode clean-up filter. In a further embodiment, the wavelength filter is adapted to attenuate spectral components having a wavelength of about 500 nm to about 1000 nm in the excitation beam, to prevent interference of the excitation beam and the reaction light. In a further embodiment, where the reaction light a wavelength of about 470 nm, the wavelength filter comprises a pass-band filter for passing light with a wavelength of about 470 nm in a band of about 5 nm. In a further embodiment, the wavelength filter is positioned after the attenuator.

Alternatively, the wavelength filter may be positioned before the attenuator. In an alternative embodiment, the optical assembly may comprise at least one wavelength filter, the or each wavelength filter for filtering spectral components in at least one of the plurality of split excitation beams from the beam splitter arrangement.

In an embodiment, the device comprises a beam splitter arrangement having one or more beam splitters, the beam splitter arrangement being configured to split the excitation beam into a plurality of split excitation beams, the or each beam splitter configured to split an incoming beam into two beams, wherein in the case where the beam splitter arrangement comprises more than one beam splitter, the beam splitters are arranged in tiers such that a first tier comprises one beam splitter for receiving the excitation beam, and each of the other tiers comprises one or more beam splitters, each beam splitter in at least one of the other tiers being configured to receive a split excitation beam from a previous tier.

In an embodiment, at least one beam splitter of said one or more beam splitters is a cube beam splitter that is configured to receive a single beam, and to split the single beam into two split beams, each split beam having substantially the same or different intensities. Alternatively, at least one beam splitter of said one or more beam splitters may be a plate beam splitter that is configured to receive one beam or a plurality of spaced apart beams, and to split the or each beam into two split beams, each split beam having substantially the same or different intensities. In a further embodiment, the beam splitter arrangement may comprise a combination of cube beam splitter(s) and plate beam splitter(s). In a further embodiment, the beam splitter arrangement comprises a plurality of beam splitters, and each beam splitter comprises a cube beam splitter. In a further embodiment, the beam splitter arrangement comprises up to about ten beam splitters. In an alternative embodiment, two or more beam splitters of the beam splitter arrangement together are a monolithic optical component.

In an embodiment, the beam splitter arrangement comprises 2ⁿ-1 number of beam splitters configured to split the excitation beam into 2ⁿ number of split excitation beams of substantially equal intensity and wavelength, n being an integer greater than zero, the or each beam splitter being configured to split an incoming beam into two beams, wherein in the case where n is more than one, the beam splitters are arranged in n number of tiers such that a first tier contains one beam splitter for receiving the excitation beam and an i^{th} tier contains 2ⁱ⁻¹ beam splitters, i being an integer ranging from 2 up to n, where the or each respective beam splitter in a tier is associated with two respective beam splitters in a next tier such that two beams split by a respective beam splitter in a tier are split further into four beams by the associated beam splitters in the next tier.

In an embodiment, the beam splitter arrangement is configured to split the excitation beam into up to k number of split excitation beams, k being an even integer greater than two, wherein the beam splitters are arranged in m number of tiers, where m is an integer greater than one and k=2×m, such that
a first tier contains one beam splitter that is configured to receive the excitation beam, and to split the incoming beam into two split excitation beams, an i^{th} tier, i being an integer ranging from 2 to m, is configured to receive incoming beams from a previous tier and to split each incoming beam into two split excitation beams, wherein in the case where i is less than m, one of the split excitation beams is directed to the next tier and the other split excitation beam is one of the k split excitation beams, and in the case where i equals m, each split excitation beam from the m^{th} tier is one of the k split excitation beams.

In an embodiment, the i^{th} tier is configured to split each incoming beam into two split excitation beams having a beam intensity of about

$$\frac{100}{m - ( i - 2 )} \%$$

and about

$$100 ( 1 - \frac{1}{m - ( i - 2 )} ) \%$$

respectively, wherein the split excitation beam with the higher intensity is directed to the next tier and the split excitation beam with the lower intensity is one of the k split excitation beams, and each split excitation beams from the m^{th} tier is one of the k split excitation beams, wherein the k split excitation beams have substantially equal intensity and wavelength.

In an embodiment, the beam splitter arrangement is configured to split the excitation beam into up to k number of split excitation beams, k being an even integer greater than two, wherein the beam splitters are arranged in (m+n) number of tiers, where m and n are integers indicating the number of primary tiers and secondary tiers respectively, m being greater than one and n being greater than zero, and k=2×m×(n+1), such that
the first tier, which is one of the primary tiers, contains one beam splitter that is configured to receive the excitation beam, and to split the incoming beam into two split excitation beams,
an i^{th} tier, which is one of the primary tiers, i being an integer ranging from 2 to m, is configured to receive incoming beams from a previous tier and to split each incoming beam into two split excitation beams, wherein in the case where i is less than m, one of the split excitation beams is directed to the next tier and the other split excitation beam is directed to the (m+1)^{th} tier, and in the case where i equals m, the split excitation beams from the m^{th} tier are directed to the (m+1)^{th} tier of the secondary tiers,
a f^{h} tier, which is one of the secondary tiers, j being an integer ranging from m+1 to m+n, is configured to receive incoming beams from a previous tier and to split each incoming beam into two split excitation beams, wherein in the case where j is less than m+n, one of the split excitation beams is directed to the next tier and the other split excitation beam is one of the k split excitation beams, and in the case where j equals m+n, each split excitation beam from the (m+n)^{th} tier is one of the k split excitation beams.

In an embodiment, the i^{th} tier is configured to split each incoming beam into two split excitation beams having a beam intensity of about

$$\frac{100}{m - ( i - 2 )} \%$$

and about

$$100 ( 1 - \frac{1}{m - ( i - 2 )} ) \%$$

respectively, wherein the split excitation beam with the higher intensity is directed to the next tier and the split excitation beam with the lower intensity is directed to the (m+1)^{th} tier, and the split excitation beams from the m^{th} tier are directed to the (m+1)^{th} tier. In a further embodiment, the j^{h} tier is configured to split each incoming beam into two split excitation beams having a beam intensity of about

$$\frac{100}{( m + n ) - ( j - 2 )} \%$$

and about

$$100 ( 1 - \frac{1}{( m + n ) - ( j - 2 )} ) \%$$

respectively, wherein the split excitation beam with the higher intensity is directed to the next tier and the split excitation beam with the lower intensity is one of the k split excitation beams, and each split excitation beam from the m+n^{th} tier is one of the k split excitation beams, wherein the k number of excitation beams have substantially equal intensity and wavelength.

In an embodiment, the beam splitter arrangement of the device for detecting molecule(s) in eighteen reaction vessel chambers has five tiers, three of which are primary tiers and two which are secondary tiers, such that
a first tier is configured to receive the excitation beam and to split the excitation beam from the collimator into two beams of substantially equal intensities,
a second tier is configured to receive two incoming beams from the first tier and to split each incoming beam into a split excitation beam of about 33% intensity and a split excitation beam of about 67% intensity,
a third tier is configured to receive the two split excitation beams of about 67% intensity from the second tier and to split each incoming beam into two split excitation beams of substantially equal intensities,
a fourth tier of the secondary tiers is configured to receive the two 33% intensity split excitation beams from the second tier and four split excitation beams from the third tier and to split each incoming beam into a split excitation beam of about 33% intensity and a split excitation beam of about 67% intensity, and
a fifth tier is configured to receive the six split excitation beams of about 67% intensity from the fourth tier and to split each incoming beam into two split excitation beams of substantially equal intensities,
   wherein the eighteen split excitation beams of substantially equal intensity and wavelength comprise six split excitation beams of about 33% intensity from the fourth tier and twelve split excitation beams from the fifth tier. In an embodiment, the reaction vessel chambers may be separately or integrally formed. In an embodiment, the first, second, third, fourth and fifth tiers comprise cube beam splitters. In an alternative embodiment, the first, second, third, fourth and fifth tiers comprise plate beam splitters. In a preferred embodiment, the first, second, third, fourth and fifth tiers comprise a combination of cube beam splitters and plate beam splitters. In a further embodiment, the first, second and third tiers comprise cube beam splitters, and the fourth and fifth tiers each comprise a plate beam splitter.

In an embodiment, the beam splitter arrangement is configured to split the excitation beam into up to k number of split excitation beams, k being an integer greater than two, wherein the beam splitters are arranged in m number of tiers, where m is an integer greater than one and k=(m+1), such that
an i^{th} tier, i being an integer ranging from 1 to m, is configured to split the excitation beam from the collimator, wherein
   in the case where i is less than m, the i^{th} tier is configured to receive an incoming beam from the collimator if i equals 1, or from a previous tier if i is more than 1, and to split the incoming beam into two split excitation beams, one of the split excitation beams is directed to the next tier and the other split excitation beam is one of the k split excitation beams, and in the case where i equals m, the m^{th} tier is configured to receive and split an incoming beam from a previous tier, and each split excitation beam from the m^{th} tier is one of the k split excitation beams.

In an embodiment, the i^{th} tier is configured to split the incoming beam into two split excitation beams having a beam intensity of about

$$\frac{100}{m - ( i - 2 )} \%$$

and about

$$100 ( 1 - \frac{1}{m - ( i - 2 )} ) \%$$

respectively, wherein the split excitation beam with the higher intensity is directed to the next tier and the split excitation beam with the lower intensity is one of the k split excitation beams, and each split excitation beam from the m^{th} tier is one of the k split excitation beams.

In an embodiment, the beam splitter arrangement is configured to split the excitation beam into up to k number of split excitation beams, k being an even integer greater than two, wherein the beam splitters are arranged in (m+n) number of tiers, where m and n are integers indicating the number of primary tiers and secondary tiers respectively, m being greater than one and n being greater than zero, and k=(m+1)×(n+1), such that
an i^{th} tier, which is one of the primary tiers, i being an integer ranging from 1 to m, is configured to split the excitation beam from the collimator, wherein
   in the case where i is less than m, the j^{th} tier is configured to receive an incoming beam from the collimator if i equals 1, or from a previous tier if i is more than 1, and to split the incoming beam into two split excitation beams, one of the split excitation beams is directed to the next tier and the other split excitation beam is directed to the (m+1)^{th} tier, and in the case where i equals m, the m^{th} tier is configured to receive and split an incoming beam from a previous tier and to direct the split excitation beams to the (m+1)^{th} tier of the secondary tiers,
a f^{th} tier, which is one of the secondary tiers, j being an integer ranging from m+1 to m+n, is configured to receive incoming beams from a previous tier and to split each incoming beam into two split excitation beams, wherein in the case where j is less than m+n, one of the split excitation beams is directed to the next tier and the other split excitation beam is one of the k split excitation beams, and in the case where j equals m+n, each split excitation beam from the (m+n)^{th} tier is one of the k split excitation beams.

In an embodiment, the i^{th} tier is configured to split the incoming beam into two split excitation beams having a beam intensity of about

$$\frac{100}{m - ( i - 2 )} \%$$

and about

$$100 ( 1 - \frac{1}{m - ( i - 2 )} ) \%$$

respectively, wherein the split excitation beam with the higher intensity is directed to the next tier and the split excitation beam with the lower intensity is directed to the (m+1)^{th} tier, and the split excitation beams from the m^{th} tier are directed to the (m+1)^{th} tier. In a further embodiment, the f^{h} tier is configured to split each incoming beam into two split excitation beams having a beam intensity of about

$$\frac{100}{( m + n ) - ( j - 2 )} \%$$

and about

$$100 ( 1 - \frac{1}{( m + n ) - ( j - 2 )} ) \%$$

respectively, wherein the split excitation beam with the higher intensity is directed to the next tier and the split excitation beam with the lower intensity is one of the k split excitation beams, and each split excitation beam from the m+n^{th} tier is one of the k split excitation beams, wherein the k number of excitation beams have substantially equal intensity and wavelength.

In an embodiment, the beam splitter arrangement of the device for detecting molecule(s) in eighteen reaction vessel chambers has seven tiers, five of which are primary tiers and two which are secondary tiers, such that
a first primary tier is configured to receive the excitation beam and to split the excitation beam from the collimator into a split excitation beam of about 17% intensity and a split excitation beam of about 83% intensity,
a second primary tier is configured to receive the split excitation beam of about 83% intensity from the first tier and to split the incoming beam into a split excitation beam of about 20% intensity and a split excitation beam of about 80% intensity,
a third primary tier is configured to receive the split excitation beam of about 80% intensity from the second tier and to split the incoming beam into a split excitation beam of about 25% intensity and a split excitation beam of about 75% intensity,
a fourth primary tier is configured to receive the split excitation beam of about 75% intensity from the third tier and to split the incoming beam into a split excitation beam of about 33% intensity and a split excitation beam of about 67% intensity,
a fifth primary tier is configured to receive the split excitation beam of about 67% intensity from the fourth tier and to split the incoming beam into two split excitation beams of substantially equal intensities,
a sixth tier, which is the first secondary tier, is configured to receive the 17% intensity split excitation beam from the first tier, the 20% intensity split excitation beam from the second tier, the 25% intensity split excitation beam from the third tier, the 33% intensity split excitation beam from the fourth tier, and two split excitation beams from the fifth tier and to split each incoming beam into a split excitation beam of about 33% intensity and a split excitation beam of about 67% intensity, and
a seventh tier, which is the second secondary tier, is configured to receive the six split excitation beams of about 67% intensity from the sixth tier and to split each incoming beam into two split excitation beams of substantially equal intensities,
   wherein the eighteen split excitation beams of substantially equal intensity and wavelength comprise six split excitation beams of about 33% intensity from the sixth tier and twelve split excitation beams from the seventh tier. In an embodiment, the first to fifth tiers are part of a primary monolithic optical assembly. In an embodiment, the sixth and seventh tiers are part of a secondary monolithic assembly. In an embodiment, the primary monolithic assembly and secondary monolithic assembly are separate components. In an alternative embodiment, the primary monolithic assembly and secondary monolithic assembly form a single component. In an embodiment, the first, second, third, fourth, fifth, sixth, and seventh tiers comprise cube beam splitters. In an alternative embodiment, the first, second, third, fourth, fifth, sixth, and seventh tiers comprise plate beam splitters. In a preferred embodiment, the first, second, third, fourth, fifth, sixth, and seventh tiers comprise a combination of cube beam splitters and plate beam splitters. In a further embodiment, the first, second, third and fourth tiers comprise cube beam splitters, and the sixth and seventh tiers each comprise a plate beam splitter.

In an embodiment, the device comprises at least one guide arrangement, the or each guide arrangement for guiding the excitation beam or a respective one of the excitation beams along an excitation path from the beam splitter arrangement into a reaction vessel containing a sample to stimulate an emission of reaction light from the sample. In a further embodiment, the or each guide arrangement is further configured to guide reaction light from the sample along a detection path towards a detector. In a further embodiment, the guide arrangement comprises an element for guiding a split excitation beam from the beam splitter arrangement to the reaction vessel and a separate element for guiding the reaction light from the reaction vessel to the detector. In a further embodiment, the guide arrangement comprises a first filter element and a second filter element positioned on or facing opposite sides of the reaction vessel, the first filter element being configured to guide a respective one of the split excitation beams along an excitation path from the beam splitter arrangement into the reaction vessel, and the second filter element being configured to guide reaction light from the sample along the detection path towards the detector. In a further embodiment, the first filter element is configured to pass the excitation beam from the beam splitter arrangement toward the reaction vessel and to reflect the reaction light from the reaction vessel. In a further embodiment, the second filter element is configured to pass the reaction light from the reaction vessel toward the detector and to attenuate or block the excitation beam. In a further embodiment, the first filter element and/or second filter element comprises a dichroic element. In a further embodiment, where a plurality of wavelengths of excitation beams are used or where the reaction light comprises multiple reaction light wavelengths, the dichroic element may be replaced by a multi-transition interference filter, such as a trichroic element, a notch filter, or a multi-bandpass filter for example.

In an embodiment, the beam splitter arrangement comprises a beam steering device for allowing the tiers to be positioned in a desired arrangement relative to each other and/or for allowing beam splitters within a tier to be positioned in a desired arrangement relative to each other. In a further embodiment, the beam steering device comprises a mirror that is substantially about 100% optically reflective.

In an embodiment, the device comprises attenuators positioned between the primary tiers and the secondary tiers for reducing the power of the split excitation beam from the primary tiers. In a further embodiment, the attenuator comprises a neutral density (ND) filter.

In an embodiment, the device comprises attenuators positioned after the secondary tiers for reducing the power of the split excitation beam from the secondary tiers. In a further embodiment, the attenuator comprises a neutral density (ND) filter.

In an embodiment, the optical assembly comprises a linear polariser. Preferably, the linear polariser trims the power (via polariser rotation) of the split excitation beams from the beam splitter arrangement such that each laser channel is then of substantially equal power. In a further embodiment, where the beam splitter arrangement is configured to produce a plurality of split excitation beams, the device comprises a plurality of linear polarisers, each linear polariser configured to receive a respective one of the split excitation beams.

In an embodiment, the device further comprises a focusing lens for focusing a respective one of the split excitation beams from the first guide into one of the reaction vessels and/or for imaging reaction light from the reaction vessel(s) to the detector. Preferably, the optical assembly further comprises a second collimator for collimating or focusing reaction light from the respective reaction vessel towards the detector. Preferably, the second collimator is a collimating lens. In an embodiment, the focusing lens is positioned between the excitation arrangement and the respective reaction vessel, and the collimating lens is positioned between the respective reaction vessel and the detector arrangement. In an alternative embodiment, the focusing lens and the collimating lens may form part of a single focusing/collimating lens. In a further embodiment, where the device is configured for a plurality of reaction vessels, the device comprises a plurality of collimating/focusing lenses, each focusing/collimating lens configured to receive reaction light from a respective one of the reaction vessels.

In an embodiment, the device comprises a glass filter for removing non-collimated excitation light components from the reaction light. In a further embodiment, the glass filter attenuates wavelengths less than about 500 nm.

In an embodiment, the detector arrangement comprises a photodiode for generating a photo-electrical current proportional to the received reaction light intensity. In a further embodiment, the photodiode is an avalanche photodiode or silicon PIN photodiode.

In a third aspect, the present invention provides a method for detection of one or more molecules in a sample contained in a reaction vessel, the reaction vessel having a portion that is substantially optically transparent to a light of at least a first range of wavelengths, the method comprising:
thermally coupling the reaction vessel to a reaction vessel holder of the device of the second aspect of the invention described above; and
guiding light to and/or from the sample in the reaction vessel through the transparent portion of the reaction vessel holder and through the transparent portion of the reaction vessel.

The device of the second aspect of the invention may have one or more features outlined in relation to the first or second aspect above.

In an embodiment, the method comprises thermally coupling the reaction vessel holder to a thermal device for heating and/or cooling the reaction vessel holder.

In an embodiment, where the thermal device is a single thermoelectric cooling unit, the method comprises thermally cycling the reaction vessel holder at up to about 20° C. per second. In a further embodiment, where the thermal device comprises two thermoelectric cooling units, the method comprises thermally cycling the reaction vessel holder at up to about 40° C. per second. In a further embodiment, where the thermal device comprises a heat source thermally coupled to the reaction vessel holder, the method comprises heating the reaction vessel holder when a temperature of the device is below a temperature threshold. The heat source may comprise a resistive heater for example that is activated when the device is cold.

In an embodiment, the method comprises physically mounting the reaction vessel holder to the thermal device. In a further embodiment, the thermal device comprises a plurality of thermoelectric cooling units, and the method comprises mounting the reaction vessel holder to the thermoelectric cooling units. In a further embodiment, the thermal device comprises two thermoelectric cooling units, and the method comprises physically mounting the reaction vessel holder to each of the thermoelectric cooling units. In a further embodiment, the method comprises mounting an end of the reaction vessel holder to one of the thermoelectric cooling unit and mounting an opposite end of the reaction vessel holder to the other thermoelectric cooling unit.

In an embodiment, the method comprises thermally coupling the reaction vessel to the reaction vessel holder using a thermal coupling medium. In a further embodiment, the thermal coupling medium comprises a thermal coupling oil.

In an embodiment, the method comprises securing the reaction vessel to the reaction vessel holder using a reaction vessel securing member. In a further embodiment, the reaction vessel securing member applies a downward force onto the reaction vessel when the reaction vessel is placed on top of the reaction vessel holder to allow for increasing the physical and thermal contact between the reaction vessel and the reaction vessel holder. In an alternative embodiment, the method comprises securing the reaction vessel to the reaction vessel holder using two reaction vessel securing members for securing opposite sides or ends of the reaction vessel. In one embodiment, the reaction vessel securing member(s) is/are moveable between a release configuration in which the reaction vessel can be positioned on or removed from the reaction vessel holder and a secure configuration in which the reaction vessel is secured to the reaction vessel holder.

In an embodiment, the method comprises securing the reaction vessel holder to the thermal device using a member. In a further embodiment, the member comprises a clamp or a fastener. In a further embodiment, the clamp has a low thermal conductivity.

In a further embodiment, where the thermal device comprises two thermoelectric cooling modules, two clamps are provided and the method comprises securing the reaction vessel holder using a respective one of the clamps to a respective one of the thermoelectric cooling unit.

In an embodiment, the method further comprises sensing a temperature of the reaction vessel holder using a temperature sensor. In a further embodiment, the temperature sensor is separate from or integral with the reaction vessel holder. In a further embodiment, the method comprises heating and/or cooling the reaction vessel holder using the thermal device based on measurements from the temperature sensor.

In an embodiment, the method comprises using a filter to pass light having the first range of wavelengths and block or reflect beams with at least a second range of wavelengths. The filter may be separate from or integral with the reaction vessel holder. In a further embodiment, the filter is an optical coating on a surface of the transparent portion of the reaction vessel holder.

In an embodiment, the method comprises generating one or more excitation beams, using an excitation arrangement, for stimulating an emission of reaction light from the sample. In a further embodiment, the method comprises detecting the reaction light from the sample using a detector arrangement. In a further embodiment, the reaction vessel comprises a further portion that is substantially optically transparent to a light of at least a second range of wavelengths, and the method comprises positioning the reaction vessel between the excitation arrangement and the detector arrangement. In a further embodiment, the excitation arrangement and the detector arrangement are on or facing opposite sides of the reaction vessel. In a further embodiment, the method comprises positioning the reaction vessel on an opposite side of the reaction vessel holder to the excitation arrangement. In an alternative embodiment, the method comprises positioning the reaction vessel on an opposite side of the reaction vessel holder to the detector arrangement.

Where specific integers are mentioned herein which have known equivalents in the art to which this invention relates, such known equivalents are deemed to be incorporated herein as if individually set forth.

In addition, where features or aspects of the invention are described in terms of Markush groups, those persons skilled in the art will appreciate that the invention is also thereby described in terms of any individual member or subgroup of members of the Markush group.

As used herein ‘(s)’ following a noun means the plural and/or singular forms of the noun.

As used herein the term ‘and/or’ means ‘and’ or ‘or’ or both.

The term ‘comprising’ as used in this specification means ‘consisting at least in part of’. When interpreting each statement in this specification that includes the term ‘comprising’, features other than that or those prefaced by the term may also be present. Related terms such as ‘comprise’ and ‘comprises’ are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents or such sources of information is not to be construed as an admission that such documents or such sources of information, in any jurisdiction, are prior art or form part of the common general knowledge in the art.

Although the present invention is broadly as defined above, those persons skilled in the art will appreciate that the invention is not limited thereto and that the invention also includes embodiments of which the following description gives examples.

### BRIEF DESCRIPTION OF THE FIGURES

Embodiments of the invention will now be described, by way of non-limiting example, with reference to the accompanying figures in which:

FIG. 1 shows a perspective view from above of an embodiment of a single sample reaction vessel for use in device of embodiments of the invention;

FIG. 2 shows a perspective view from below of the reaction vessel shown in FIG. 1;

FIG. 3 shows a perspective view of an embodiment of an eighteen sample reaction vessel for use in the device of embodiments of the invention;

FIG. 4 shows a front view of components of the device according to an embodiment of the present invention;

FIG. 5 shows a perspective view of components of the device according to an alternative embodiment of the invention;

FIG. 6 shows a control system block diagram of the device according to an embodiment of the present invention;

FIG. 7 shows a perspective view of a device according to an embodiment of the present invention in a closed configuration;

FIG. 8 shows a perspective view of the device shown in FIG. 7 in an open configuration;

FIG. 9 shows a perspective view of the layout of components of the device shown in FIG. 7 in the closed configuration;

FIG. 10 shows a side view of the layout of components of the device shown in FIG. 7 in the closed configuration;

FIG. 11 shows part of the optical assembly of the device according to an embodiment of the present invention;

FIG. 12 shows a first example schematic beam splitter arrangement of the device according to an embodiment of the present invention;

FIG. 13 shows a second example schematic beam splitter arrangement of the device according to an embodiment of the present invention;

FIG. 14 shows a third example schematic beam splitter arrangement of the device according to an embodiment of the present invention;

FIG. 15 shows a fourth example schematic beam splitter arrangement of the device according to an embodiment of the present invention;

FIG. 16 shows a fifth example schematic beam splitter arrangement of the device according to an embodiment of the present invention;

FIG. 17 shows part of the optical assembly of the device according to an embodiment of the present invention;

FIG. 18 shows a perspective view of a first example device according to an embodiment of the present invention;

FIG. 19 shows an exploded view of the device shown in FIG. 18;

FIG. 20 shows a perspective view of the optical and thermal device housing of the device shown in FIG. 18;

FIG. 21 shows an exploded view of the housing shown in FIG. 20 with the guide housing;

FIG. 22 shows the primary tiers housing of the device shown in FIG. 18;

FIG. 23 shows the secondary tiers housing of the device shown in FIG. 18;

FIG. 24 shows a perspective view of a second example device according to an embodiment of the present invention in a closed configuration;

FIG. 25 shows a perspective view of the device shown in FIG. 24 in an open configuration;

FIG. 26 shows a perspective view from the top of the lower housing of the device shown in FIG. 24;

FIG. 27 shows a perspective view from the bottom of the upper housing of the device shown in FIG. 24;

FIG. 28 shows a perspective view of the optical and thermal device housing of the device shown in FIG. 24 with the reaction vessel securing members in the release configuration;

FIG. 29 shows a perspective view of the optical and thermal device housing of the device shown in FIG. 24 with the reaction vessel securing members in the secure configuration;

FIG. 30 shows a perspective view of the optical and thermal device housing and components in the upper casing of the device shown in FIG. 24;

FIG. 31 shows a perspective view of the device shown in FIG. 30 with the optical housing in the upper casing removed;

FIG. 32 show a perspective view of the device shown in FIG. 31 with the reaction vessel removed;

FIG. 33 shows a perspective view of the device shown in FIG. 32 with the component casing removed;

FIG. 34 shows a perspective view of the device shown in FIG. 33 with the reaction vessel securing members removed;

FIG. 35 shows a plot of LC480 quantitative DNA amplification for six samples;

FIG. 36 shows a plot of 1sHHD quantitative DNA amplification for six samples;

FIG. 37 shows an agarose gel electrophoresis using the LC480 and the 1sHHD;

FIG. 38 shows a plot of percentage fluorescence over a serial dilution for different temperatures;

FIG. 39 shows a plot for determining DNA concentration of a sample from the fluorescence using the 1sHHD;

FIG. 40 shows the microscopy images of two dilutions of GFP protein biobeads;

FIG. 41 shows a plot of the average percentage fluorescence in response to temperature;

FIG. 42 shows a schematic of the 18-sample hand held device according to an embodiment of the present invention;

FIG. 43 shows the excitation beams output from the device of FIG. 42 on a sheet of paper;

FIG. 44 shows a perspective view of an embodiment of a three-sample reaction vessel for use in device of embodiments of the invention; and

FIG. 45 shows an agarose gel electrophoresis using the GA and the diamond thermal plate of the 1sHHD.

### DETAILED DESCRIPTION OF THE INVENTION

Embodiments of the present invention relate to a compact handheld portable device for detecting molecule(s). The device may be suitable or configured for amplification and detection of nucleic acids in a sample. For example, the device could be used for polymerase chain reaction (PCR) analysis (including quantitative PCR analysis). The device may additionally or alternatively be suitable or configured for one or more of: protein analysis, ligand analysis, or fluorescence analysis from any chemical reaction for example. Further, the device may be used for detecting molecule(s) within a single reaction vessel, or in a plurality of samples from the same or different sources within a plurality of reaction vessels, or a single reaction vessel with a plurality of samples.

The device of an embodiment of the present invention generally comprises a reaction vessel holder for receiving one or more reaction vessels containing a sample. A thermal device, which may be a heater/cooler or a heat exchange device, is coupled to the reaction vessel holder to control the temperature of the sample within the reaction vessel(s). For example, the thermal device is configured to increase and/or decrease the temperature of the sample within the reaction vessel(s) held by the reaction vessel holder and/or to maintain the temperature at a desired level. Where the device is suitable or configured for amplification and detection of nucleic acids, the process of heating and/or cooling the samples within the reaction vessels in one or more stages results in amplification of the nucleic acids in the sample.

The device further comprises an optical assembly for detection of the molecule(s) in the sample(s) within the reaction vessel(s). Generally, the optical assembly is configured to transmit a beam of excitation radiation toward the reaction vessel(s) which stimulates an emission of a reaction light such as fluorescence from the sample within the reaction vessel(s). The optical assembly is further configured to receive the reaction light from the reaction vessel(s). The optical assembly may be coupled to a controller that is configured to control the heating/cooling operation of the reaction vessel excitation radiation operation, and the reaction light detection operation. The optical assembly need not be a single grouping of components on or facing one side of the reaction vessel, and may instead comprise two (or more) groupings of components positioned on opposite sides of the reaction vessel.

These components will be described in further detail below.

The Reaction Vessel

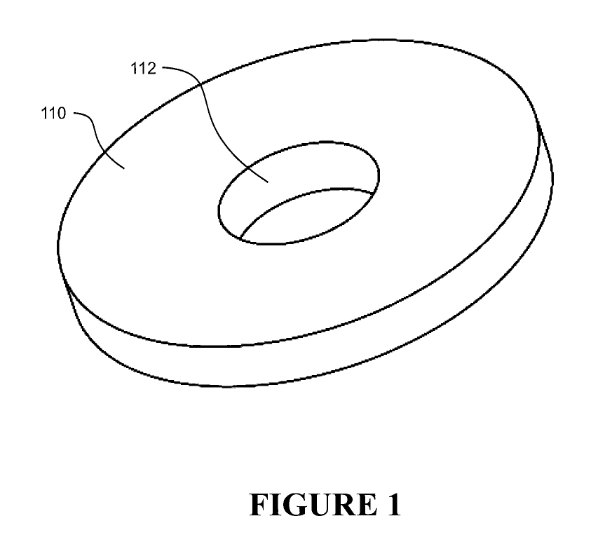

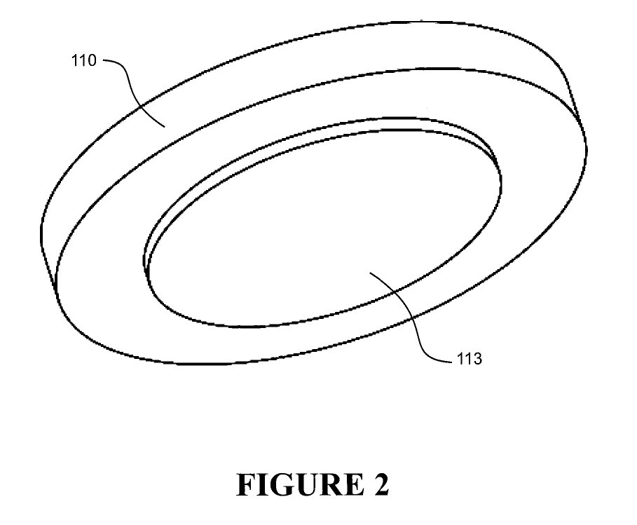

An example of the reaction vessel 110 for a single sample is shown in FIGS. 1 and 2. The reaction vessel is substantially disc-shaped. The reaction vessel comprises a reaction chamber 112, which is configured to receive the sample for molecule detection. The reaction vessel 110 comprises a substantially optically transparent portion. Preferably, at least the base 113 of the reaction vessel 110, as shown in FIG. 2, is the substantially optically transparent portion of the reaction vessel 112. Alternatively, the whole reaction vessel 110 may be substantially optically transparent.

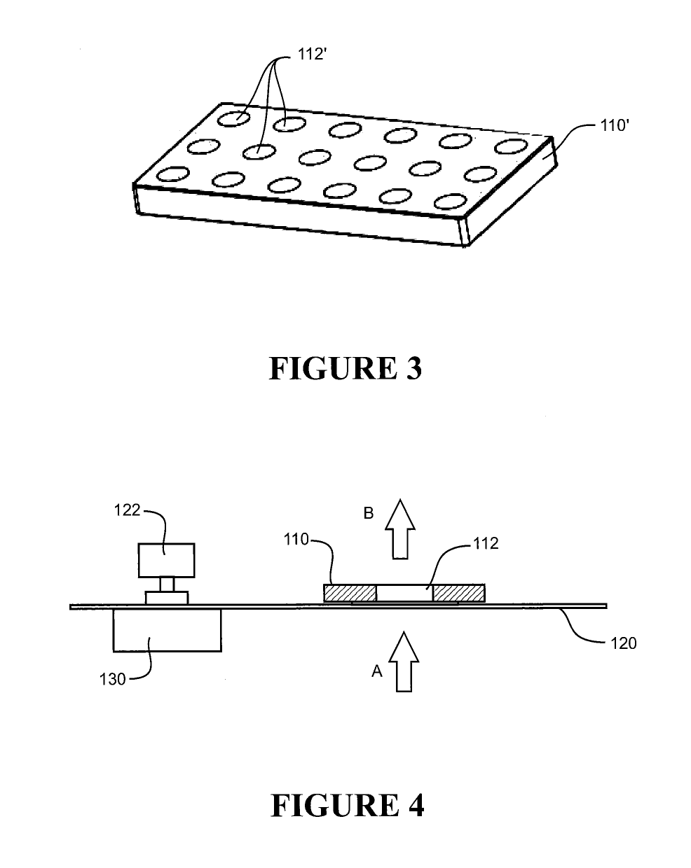

An alternative example reaction vessel 110′ which contains the sample to be analysed is shown in FIG. 3. The reaction vessel 110′ is in the form of a cassette housing. Features of the reaction vessel 110′ shown in FIG. 3 will be described below. Other than the difference in number of reaction chambers, features of the eighteen sample reaction vessel 110′ are similar to the features of the single sample reaction vessel 110.

The reaction vessel 110′ may be low cost and be disposable.

The reaction vessel 110′ is provided with eighteen reaction chambers or wells 112′. Each reaction chamber 112′ has a low volume and is configured to receive a sample for molecule detection. Each reaction chamber 112′ is configured so that no air gap is present above the reaction mixture into which the reaction mixture may evaporate. According to other embodiments, the reaction vessel may be provided with more than one or more than eighteen reaction chambers. The device comprises a dedicated excitation beam and associated optics for sample fluorescence detection for each reaction chamber 112′ of the reaction vessel 110′.

The reaction chambers 112′ may each have a parabolic shape to direct reaction light toward the detector arrangement. A base of the parabola for each reaction chamber 112′ at a bottom of the reaction chamber 112′ may be substantially flat to allow convergent excitation light and/or reaction light to pass through the bottom without being refocused by the bottom of the reaction chamber 112′.

The reaction vessel 110′ is preferably optically transparent on at least a bottom side and on an opposite top side to allow an excitation light to enter the reaction chamber that stores a sample and/or to allow a reaction light to exit the reaction chamber. According to an embodiment, the reaction vessel 110′ is optically transparent on the bottom side to allow an excitation light to enter the reaction chamber 112′ and on a top side to allow a reaction light to exit the reaction chamber 112′. According to another embodiment, the reaction vessel 110′ is optically transparent on the top side to allow an excitation light to enter the reaction chamber 112′ and on a bottom side to allow a reaction light to exit the reaction chamber 112′. According to other embodiment, a bottom and/or top side of reaction vessel 110′ may comprise at least one portion, such as a face or a window, which is substantially optically transparent to light of at least a first range of wavelengths, the first range of wavelengths comprising wavelengths of the excitation beam and/or wavelengths of the reaction light.

A cover may be provided to close one or more of the reaction chambers 112′. The cover is used to substantially seal the mouth of the reaction chambers 112′ during the heating/cooling process to prevent the sample from evaporating outside the reaction chamber 112′ when heated by the heater/cooler which would contaminate the device and affect future results. The cover may be a separate component from the reaction vessel 110′. For example, the cover may be a thin transparent sheet which covers the top side of the reaction vessel 110′. Alternatively, the cover may be for example, moveably connected to the body. The cover may be substantially transparent or translucent so that an optical path can be established between the sample and the optical assembly.

In one embodiment, the reaction chambers 112′ in the reaction vessel 110′ are separable from each other. According to further embodiments, the reaction chambers 112′ may be frangibly connected to each other to form the reaction vessel. For example, a row of reaction chambers may be frangibly separated from other reaction chambers.

Other sample reaction vessel arrangements are possible. For example, a bottom of the reaction chamber may have a reflective coating to reflect the reaction light away from the excitation arrangement and towards the detector arrangement. Further, walls of the reaction chamber may be white or reflective to reflect the reaction light away from the excitation arrangement and towards the detector arrangement. In preferred embodiments of the invention, the reaction chamber is configured to receive excitation light from an excitation arrangement positioned one side of the reaction vessel and to reflect reaction light to a detector arrangement positioned on an opposite side of the reaction vessel. These embodiments where the reaction vessel is sandwiched between the excitation arrangement and detector arrangement provide a particularly compact device.

The reaction vessel 110′ may be made from a plastic material such as acetal (0.230 Wm⁻¹K⁻¹), acrylic (0.19 Wm^{-l}K⁻¹) or polycarbonate (0.2 Wm^{-l}K⁻¹) for example. Thermally enhanced polymers may also be used to form the reaction vessel. The reaction vessel 110′ may alternatively or additionally comprise a synthetic diamond material. For example, a bottom of the reaction vessel 110′ may be impregnated with synthetic diamond particulate.

Dimensions of the reaction vessel 110′ are determined by and commensurate with the available optically transparent portion(s) of the reaction vessel holder and the desired pitch and placement of the individual reaction chambers 112′ within the reaction vessel 110′. Where the reaction vessel comprises a plurality of reaction chambers, two or more adjacent reaction chambers may be combined to form a single larger reaction chamber. In one embodiment, the reaction vessel 110′ has dimensions of 28 mm×14 mm×2 mm and an associated volume of 784 mm³. An individual reaction chamber 112′ has a typical diameter of 2 mm and a volume of approximately 6.3 mm³ (6.3 microlitres).

The reaction vessel 110′ has a low mass to allow for rapid heating and/or cooling of the samples within the reaction chambers 112′.

The Reaction Vessel Holder

An example of the reaction vessel holder 120 with a reaction vessel 110 with a single reaction chamber 112 on a thermal device 130 is shown in FIG. 4. The reaction vessel 110 may be one of the reaction vessels described with reference to FIGS. 1 and 2, for example. The reaction vessel holder 120 is an optically transparent plate that is configured to receive and support the reaction vessel 110 and that is configured to facilitate thermal transfer between the thermal device 130 and the sample within the reaction chamber 112.

The reaction vessel holder 120 is substantially formed of a synthetic diamond material. Synthetic diamond is very tough, does not oxidize and is simple to clean without scratching. As an alternative to or in addition with synthetic diamond, the reaction vessel holder may comprise synthetic sapphire or substantially optically transparent aluminium nitride for example.

According to preferred embodiments of the device, the reaction vessel holder is formed of a single unitary piece of material. In these embodiments, the reaction vessel holder is not formed of multiple layers. In some embodiments, an optical coating may be provided on a surface of the reaction vessel holder.

Synthetic diamond is a laboratory-created diamond, laboratory-grown diamond, cultured diamond or cultivated diamond. Synthetic diamond may be formed from a chemical vapour deposition (CVD) formation process. Alternatively, synthetic diamond may be formed from a high-pressure high-temperature (HPHT) formation process. Synthetic diamond has a small amount of absorption in the visible spectrum. However, there is no significant auto fluorescence under excitation laser wavelengths. Synthetic diamond has a broad optical transparency from the deep ultraviolet region to the far infrared region of the electromagnetic spectrum. Absorption by the reaction vessel holder is not an issue as the absorption is substantially uniform and can be compensated for as a surplus of excitation light is available. Small amounts of background fluorescence from sample reaction vessels are typical and small in comparison to the PCR fluorescence. Reaction light passing though the reaction vessel holder is attenuated by about 10%. This is not a significant issue as reaction light can collected directly from the sample. According to other embodiment, the reaction light may be collected through the reaction vessel holder.

Synthetic diamond has an extreme mechanical hardness of about 90 GPa. Synthetic diamond is the strongest known material with a bulk modulus of about 1.2×10¹² Nm⁻² and a compressibility of about 8.3×10⁻¹³ m²N⁻¹. Synthetic diamond is additionally a good electrical insulator. At room temperature, synthetic diamond has a resistivity of about ˜10¹⁶ Ωcm. Synthetic diamond can be doped to change its resistivity from about 10 to 10⁶ Ωcm, and to become a semiconductor with a wide bad gap of 5.4 eV. Synthetic diamond is resistant to chemical corrosion and is stable. Synthetic diamond does not react with the sample within the reaction chamber of the reaction vessel. Further, synthetic diamond exhibits a low or ‘negative’ electron affinity.

The reaction vessel holder 120 has a high thermal conductivity. The high thermal conductivity means that temperature gradients will not present any issues in the device. Synthetic diamond has a thermal conductivity of about five times more than the thermal conductivity of copper. Depending on the grade of synthetic diamond, the thermal conductivity of the reaction vessel holder 120 can be more than 1800 Wm⁻¹K⁻¹. In one embodiment, the thermal conductivity of the reaction vessel holder 120 is higher than about 1500 Wm⁻¹K⁻¹. In one embodiment, the thermal conductivity of the reaction vessel holder 120 is between about 1800 Wm⁻¹K⁻¹ and about 2100 Wm⁻¹K⁻¹.

The reaction vessel holder 120 comprises a low thermal mass. The thermal mass refers to the ability of an object to retain heat. An object with a low thermal mass has a low heat capacity or low specific heat capacity, and requires little heat to increase the temperature of the object. The object will have a low mass.

The reaction vessel holder 120 comprises a lower specific heat capacity or volumetric heat capacity compared to metals, which allows the reaction vessel holder to reach a specific temperature without requiring as much heat. At about 300K, synthetic diamond has a specific heat capacity of about 0.502 Jg⁻¹K⁻¹. In one embodiment, the specific heat capacity of the reaction vessel holder is less than about 1.0 Jg⁻¹K⁻¹ at about 300K. In one embodiment, the specific heat capacity of the reaction vessel holder is less than about 0.6 Jg⁻¹K⁻¹ at about 300K.

The reaction vessel holder 120 has a low thermal expansion coefficient. At about 300K, the thermal expansion coefficient of the reaction vessel holder 120 is between about 0.8×10⁻⁶ K⁻¹ and about 1.2×10⁻⁶ K⁻¹. Preferably, the reaction vessel holder has a thermal expansion coefficient of about 1.0×10⁻⁶ K⁻¹. Synthetic diamond has a lower coefficient of thermal expansion compared to metals. A low thermal expansion allows for reusability and longevity of the reaction vessel holder.

Instead of synthetic diamond, the reaction vessel holder 120 may comprise synthetic sapphire (Corundum or Al₂O₃) for example, which is substantially optically transparent for a range of wavelengths. A reaction vessel holder with synthetic sapphire is suitable for molecule detection applications that only require slow thermal adjustments or cycling. The thermal conductivity of synthetic sapphire is about 25 Wm^{-l}K⁻¹. The specific heat capacity of synthetic sapphire is about 0.418 Jg^{-l}K⁻¹. The thermal expansion coefficient of the synthetic sapphire is less than about 6×10⁻⁶ K⁻¹ at about 300K, such as about 5.8×10⁻⁶ K⁻¹.

The reaction vessel holder 120 may comprise a material having a thermal conductivity of about 25 Wm⁻¹K⁻¹ or higher. The material may have a specific heat capacity of less than about 1 Jg⁻¹K⁻¹. The material may have a thermal expansion coefficient of less than about 1×10^{-s} K⁻.

The reaction vessel holder 120 may alternatively comprise aluminium nitride (AlN) for example, which is substantially optically transparent for a range of wavelengths.

Aluminium nitride has a thermal conductivity of about 285 Wm⁻¹K⁻¹. The specific heat capacity of aluminium nitride is about 0.74 Jg⁻¹K⁻¹ at about 300K. The thermal expansion coefficient of aluminium nitride is between about 4×10⁻⁶ K⁻¹ and about 6×10⁻⁶ K⁻¹ at about 300 K.

The reaction vessel holder 120 may comprise a material having a thermal conductivity of about 285 Wm⁻¹K⁻¹ or higher. The material may have a specific heat capacity of less than about 0.8 Jg⁻¹K⁻¹ at about 300K. The material may have a thermal expansion coefficient of less that about 5.5×10⁻⁶ K⁻¹ at about 300K.

The reaction vessel holder 120 has a mass of between about 1 g and about 10 g. Preferably, the reaction vessel holder has a mass of between about 1 g and about 5 g, more preferably of between about 1 g and about 2.5 g, more preferably of between about 1 g and about 2 g, and more preferably of approximately 1.9 g.

The reaction vessel holder 120 is an optically transparent plate. The plate is substantially optically transparent to light of at least the first range of wavelengths. According to other embodiments, the reaction vessel holder may comprise one or more portions that are optically transparent, and one or more portions that are not optically transparent. Synthetic diamond is transmissive for wavelengths higher than about 225 nm. Where the device is configured for molecule detection in up to eighteen reaction chambers, the dimensions of the plate are 45 mm×15 mm×0.8 mm, with a density of about 3.515 gcm⁻³ for example. The dimensions depend on the number of reaction chambers that the device is configured to perform molecule detection on.

In use, the optically transparent bottom side of the reaction vessel 110 faces and rests on the reaction vessel holder 120 such that light of the first range of wavelengths to and/or from the sample in the reaction chamber 112 can pass through the reaction vessel holder 120. In the embodiment shown in FIG. 2, the light of the first range of wavelengths comprises excitation wavelengths from an excitation arrangement and has an excitation optical path A that enters through a bottom side of the reaction chamber 112 through the reaction vessel holder 120. The reaction light along from a top side of the reaction chamber 112 passes to a detector arrangement along a reaction light optical path B. The excitation optical path A and the reaction light optical path B are substantially collinear or parallel. The excitation arrangement and the detector arrangement are positioned on or facing opposite sides of the reaction vessel 110.

In an alternative embodiment, the first range of wavelengths comprises a range of reaction light wavelengths of reaction light from the sample, and reaction light from the sample is configured to pass from a bottom side of the reaction chamber through the reaction vessel holder to the detector arrangement. In that embodiment, the excitation beam from the excitation source enters the reaction chamber from a top side of the reaction vessel.

The reaction vessel holder 120 may comprise a filter for passing excitation light and for blocking or reflecting reaction light beams, or vice versa depending on the configuration of the device. The filter may be separate from or integral with the reaction vessel holder. For example, the filter may be an optical coating on a surface of the reaction vessel holder.

In one embodiment, the range of excitation wavelengths and the range of reaction light wavelengths are dependent on a dye used in the sample, and comprise the following excitation wavelength and reaction light wavelength when the following dyes are used (dye—excitation wavelength in nm—reaction light wavelength in nm): SYBR—497-520; FAM—495-520; TET—521-536; JOE—520-548; VIC—538-554; HEX—535-556; R6G—524-557; Cy3—550-570; TAMRA—555-576; NED—546-575; Cy3.5—581-596; ROX—575-602; Texas Red—583-603; Cy5—649-670; Cy5.5-675-694.

The at least one transparent portion of the body is substantially optically transparent to light having wavelengths between about 400 nm and about 800 nm. In a further embodiment, said at least one transparent portion of the body is substantially optically transparent to light having wavelengths greater than about 225 nm.

The reaction vessel holder 120 is thermally coupled to the reaction vessel 110. The reaction vessel 110 sits on top of the reaction vessel holder 120. The reaction vessel 110 may be thermally coupled to the reaction vessel holder 120 using a thermal coupling medium. The thermal coupling medium may comprise a thermal coupling oil for example. Alternatively or additionally, the device comprises a reaction vessel securing member for securing the reaction vessel 110 to the reaction vessel holder 120. The reaction vessel securing member applies a downward force onto the reaction vessel 110 when the reaction vessel is placed on top of the reaction vessel holder 120 for increasing the physical and thermal contact between the reaction vessel 110 and the reaction vessel holder 120. In some embodiments, the device comprises two reaction vessel securing members for securing opposite sides or ends of the reaction vessel 110. The reaction vessel securing member(s) may be moveable between a release configuration in which the reaction vessel 110 can be positioned on or removed from the reaction vessel holder 120 and a secure configuration in which the reaction vessel 110 is secured to the reaction vessel holder 120.

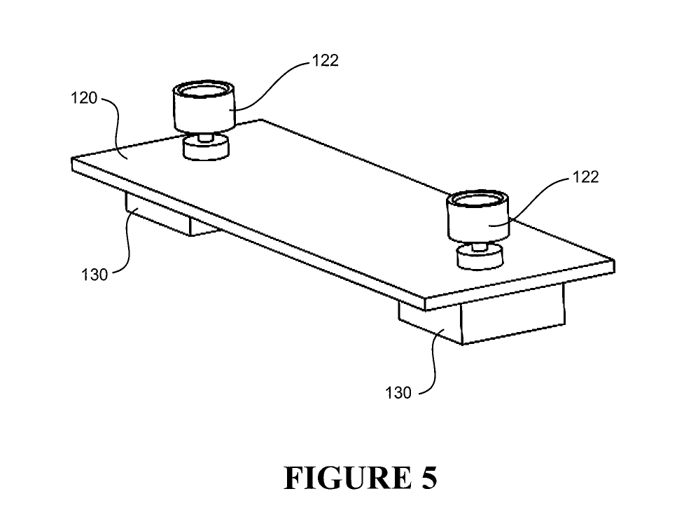

The reaction vessel holder 120 is further thermally coupled to a thermal device 130 for heating or cooling the reaction vessel holder. Due to the thermal properties of the reaction vessel holder 120 and due to the low mass of the reaction vessel holder 120 and reaction vessel 110, heat can be rapidly transferred between the thermal device 130 and reaction vessel 110. Where the device is a Q-PCR device, the reaction vessel holder 120 can be thermocycled rapidly. In the embodiment shown in FIG. 5, the thermal device 130 comprises two thermoelectric cooling units or Peltier devices physically connected to opposite ends of the reaction vessel holder 130. In this embodiment, the reaction vessel holder 120 can be thermally cycled at up to about 40° C. per second. Where the reaction vessel holder 120 is coupled to a single thermoelectric cooling unit, the reaction vessel holder 120 can be thermally cycled at up to about 20° C. per second.

One or more members in the form of clamps 122 may be provided to secure the reaction vessel holder 120 to the thermal device 130. The device may comprise one or more clamps. For example, referring to FIG. 5, two clamps are provided which secure the reaction vessel holder 120 to two thermoelectric cooling units of the thermal device 130.

Each clamp is located centrally above a respective one of the thermoelectric cooling units. Alternatively, the members may be fasteners. The members comprise a plastic material. The clamps 122 have a low thermal conductivity of about 0.2 Wm^{-l}K⁻¹. The clamps 122 are configured to secure the reaction vessel holder 120 to the thermal device 130 by applying a downward force on the reaction vessel holder 120. The clamps have a narrow cross sectional area in at least one portion of the clamp in order to further restrict heat flow via the clamp.

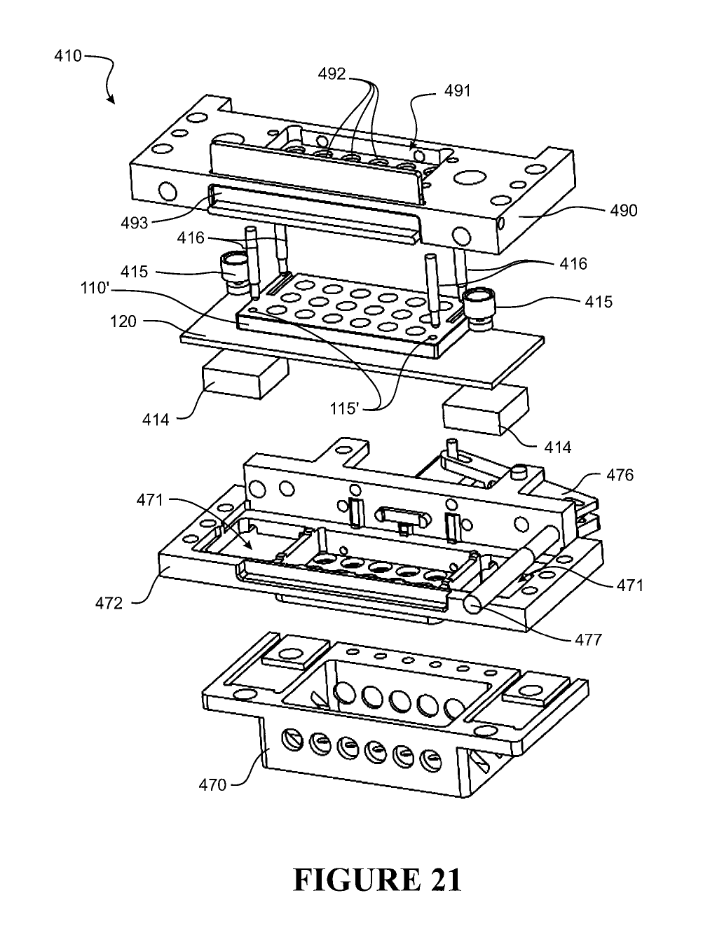

The reaction vessel holder 120 sits on top of the thermal device 130, which are two thermoelectric cooling units, each unit being positioned at either end of the reaction vessel holder 120. The housing of the device 472 (as shown in FIG. 21) is used to horizontally constrain the reaction vessel holder 120, to prevent horizontal movement. The clamps vertically constrain the reaction vessel holder 120 to the thermal device 130 using two pressure pads of low thermal conductivity. In some embodiments of the device, there may be more than two pressure pads. In addition, with the vertical force from the reaction vessel which sits on top of the reaction vessel holder 120 and which may be further pushed down onto the reaction vessel holder 120, vertical movement of the reaction vessel holder is prevented. The thermoelectric cooling units are located on the opposite side of the diamond plate directly under the pressure pads.

A temperature sensor may be provided for sensing the temperature of the reaction vessel holder 120. The temperature sensor may be a resistive thermal device for example. The temperature sensor may be separate from or integral with the reaction vessel holder 120. In one embodiment, the temperature sensor is in electronic communication with a controller for controlling an operation of the thermal device 130 for heating and/or cooling the reaction vessel holder 120. The temperature sensor comprises a resistive temperature detector, such as a 100 ohm platinum resistive temperature detector for example.

Thermal Device

Still referring to FIGS. 4 and 5, the thermal device 130, which may be a heater/cooler or a heat exchange device, is for actively heating and/or cooling the sample in one or a plurality of stages. The thermal device generally comprises a thermoelectric cooling (TEC) unit and a heat sink/fan.

A TEC unit is generally a substantially planar solid-state device which uses the Peltier effect to transfer heat from one side of the device to the other upon application of a direct current (DC) voltage. By reversing the direction of the current, the direction of heat transfer can similarly be reversed.

The TEC unit is ideal for use in the portable device of preferred embodiments of the present invention where the temperature of the sample must be alternately heated and cooled such as for example in a Q-PCR analysis. The TEC unit has no moving parts, is relatively small and lightweight, can be easily powered by battery or a relatively low-voltage source, and can both heat and cool the sample in the reaction vessel.

A first side of the substantially planar TEC unit is in direct physical contact with the base of the reaction vessel holder 120 for an efficient thermal coupling therebetween. Alternatively, the first side of the TEC unit may be indirectly connected to the base of the reaction vessel holder 120. In that alternative configuration, the TEC unit is still in substantial thermal communication with the reaction vessel holder 120. A thermal coupling medium is used to thermally couple the reaction vessel holder 120 to the thermoelectric cooling units. The thermal coupling medium may comprise a heat sink paste, such as a silver compound heat sink paste for example. Additionally or alternatively, the thermal coupling medium may comprise indium foil, which is not prone to ‘drying out’. The second, opposing, side of the TEC unit is in physical contact with a heat sink. The heat sink may for example comprise a metallic mass and a fan to actively dissipate an excess of heat. The heat sink dissipate(s) heat from the second side of the TEC unit when the sample is cooled, and provides a source of heat when the sample is heated.

The heat sink may be thermally coupled with the exterior casing of the device, which may also act as a heat sink/source. The casing is preferably a metallic material having a relatively high thermal conductivity, typically less than that of the heat sink and/or reaction vessel holder. For example, the casing may have a thermal conductivity between about 12 Wm⁻¹K⁻¹ and about 240 Wm⁻¹K⁻¹. Suitable materials for the casing may include stainless steel or aluminium for example.

Each TEC unit could be a single- or two-stage TEC unit. Alternatively, the TEC unit could be a multiple-stage TEC unit, comprising three or more stages. The device shown in FIG. 3 is a two-stage TEC unit with two TEC units positioned on opposite sides of the reaction vessel holder 102. In this configuration, the reaction vessel holder 120 can be thermally cycled at up to about 40° C. per second. Where the reaction vessel holder 120 is coupled to a single thermoelectric cooling unit, the reaction vessel holder 120 can be thermal cycled at up to about 20° C. per second.

A typical single stage TEC unit has a maximum temperature differential of approximately 70° C. TEC unit temperature differentials are additive and therefore a two-stage TEC unit has a maximum temperature differential of approximately 140° C. For the device of the present invention to operate reliably in the field, it must be capable of operating reliably and consistently in a wide range of environmental conditions and ambient temperatures.

In another embodiment of the invention, the device may be provided with a further TEC unit between the heat sink and the apparatus casing. In this configuration, the heat sink becomes a thermal reservoir and the further TEC unit is adapted to maintain the heat sink at a substantially constant temperature, preferably in the region of 30-40° C., while the first TEC unit is adapted to vary the temperature of the vessel receptacle by transferring heat between the heat sink (thermal reservoir) and the vessel receptacle 121 as necessary.

In either the single- or two-stage configuration, as appropriate, either or both of the ‘first’ and ‘further’ TEC units may actually comprise a plurality of independently controlled TEC units, typically provided side-by-side in a plane. In a preferred embodiment of a device having four vessel receptacles, the ‘first’ TEC units comprises three two-stage TEC units in parallel, with the vessel receptacles thermally coupled to the TEC units. The TEC units are further thermally coupled to a unitary thermal reservoir or heat sink.

The thermal device may additionally comprise a heat source thermally coupled to the reaction vessel holder, which is activated when a temperature of the device is below a temperature threshold. The heat source provides active heating of a chassis of the device when the device is used in a cold environment. Alternatively, the thermoelectric unit(s) could be supplemented with standard resistive/ohmic type heaters coupled to the reaction vessel holder. The heat source reduces the time taken for the device to reach desired operating temperatures.

The operation of the thermal device 130 is controlled by a control system which is described in further detail below.

The Control System

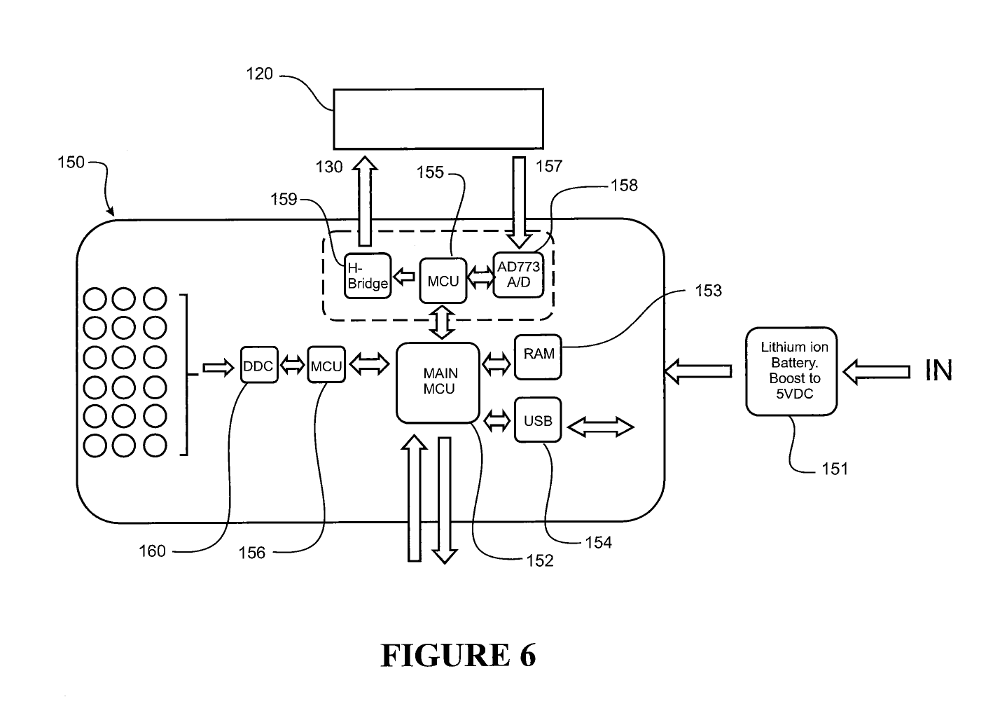

The control system 150 of the device according to an embodiment of the present invention is shown in FIG. 6.

The device is powered by a power source 151. The power source 151 may be a Lithium ion battery, with a boost converter to provide a steady 5V output voltage. The power source 151 may be a rechargeable power source by providing a charging voltage IN to the power source 151.

The device comprises a microcontroller unit 152 that is in communication with a random access memory (RAM) 153 and a USB interface 154. The microcontroller 152 is further configured to control the excitation arrangement for transmitting an excitation beam with feedback control. The microcontroller 152 is in further communication with a microcontroller unit 155 for temperature control and a microcontroller unit 156 for reaction light detection.

The microcontroller unit 155 for temperature control receives inputs from a temperature sensor device 157, which measures the temperature of the reaction vessel holder 120. An analogue-to-digital converter circuit 158 is provided to convert analogue measurements from the temperature sensor device 157 into digital inputs for the microcontroller 155. Based on the temperature measurements, the microcontroller unit 155 is configured to adjust the operation of the thermal device 130 accordingly. The thermal device is part of an H-bridge circuit 159.

The microcontroller unit 156 for reaction light detection is in communication with a multichannel charge integration integrated circuit (Texas Instruments DDC232) 160. Use of the DDC232 eliminates the need for potentially temperamental and electrically noisy trans-impedance amplifiers for the detector system. The DDC232 receives and integrates the electrical photo-charge originating from one or more photodiodes as a result of the incident reaction light upon said photodiodes.

It will be appreciated by those skilled in the art that the control system according to the embodiments of the present invention may be implemented purely in hardware consisting of one or more components which may include discrete electronic components or integrated circuits. Alternatively, or additionally, the control system of embodiments of the present invention may be implemented at least in part using programmable hardware components, such as programmable logic devices (PLDs) or field programmable gate arrays (FPGAs), or by software executed by a computing means or processor which may include the microcontroller or a general purpose personal computer (PC) programmed accordingly. Typically, however, the invention would be implemented as an embedded system using a combination of the aforementioned components, as described herein. In particular, the functions of the control system are distributed among a number of integrated circuits of the embedded system, such as the thermal device control module, battery management module, thermal management module, LED control module, and microcontroller, for example, but may alternatively be performed centrally by a single integrated or discrete circuit (such as microcontroller) without departing from the scope of the invention.

Device for Molecule Detection in a Single Reaction Chamber

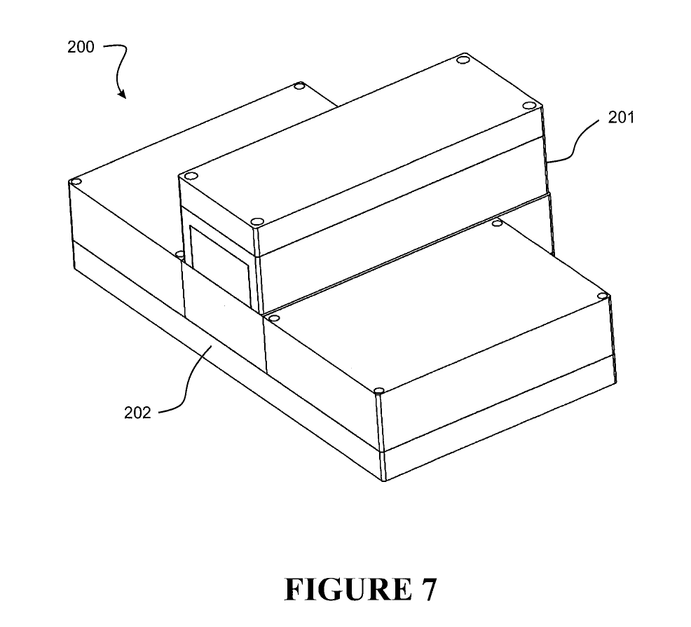

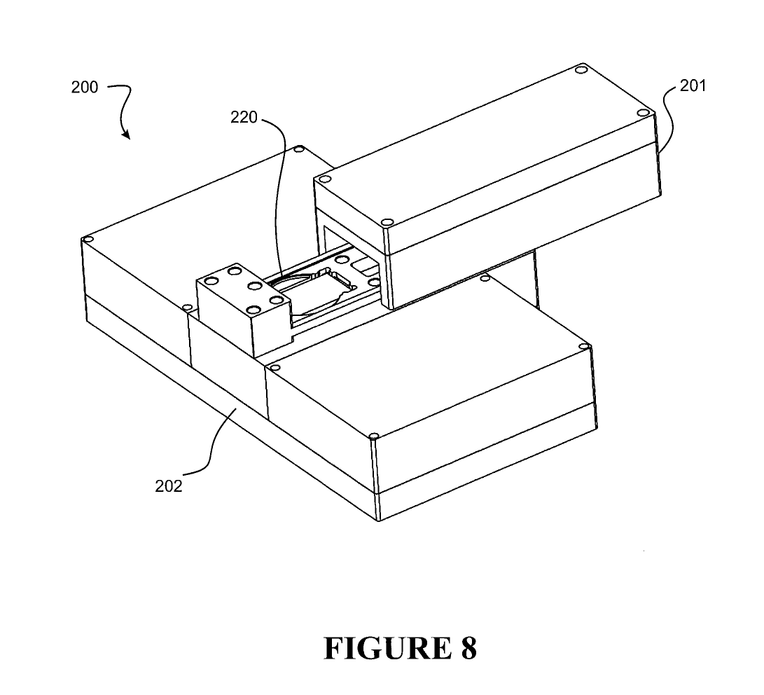

FIGS. 7 and 8 show an example of a device 200 for molecule detection in a reaction vessel 110 having a single reaction chamber 112 (as described with reference to FIGS. 1 and 2) according to an embodiment of the present invention. The device 200 comprises an upper housing 201 and a lower housing 202. The upper housing 201 is moveable relative to the lower housing 202 between a closed configuration (shown in FIG. 7) and an open configuration (shown in FIG. 8). The reaction vessel holder 120 is housed in the lower housing 202.

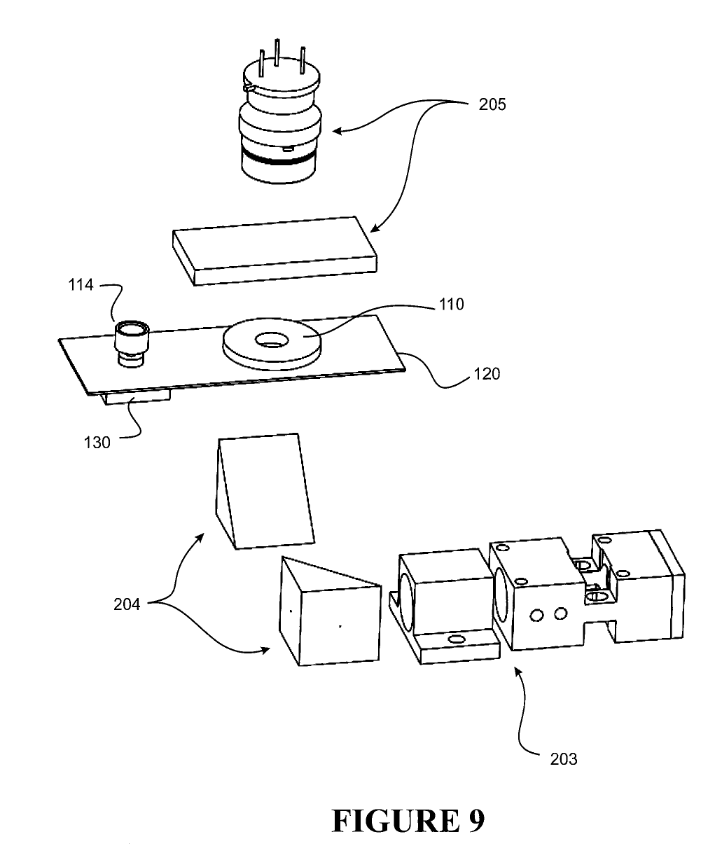

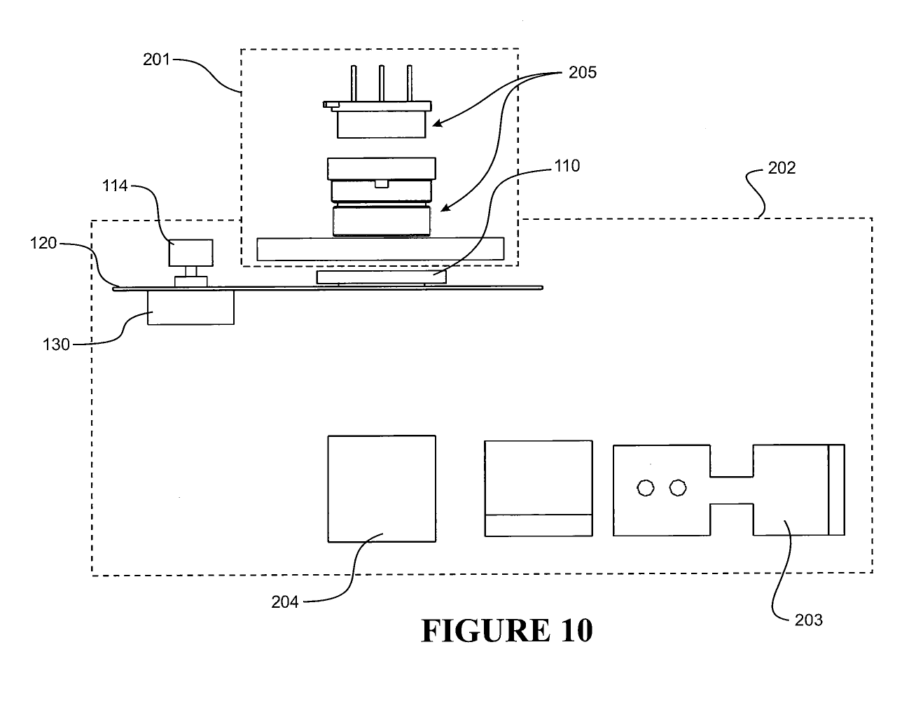

FIGS. 9 and 10 show the general layout of the components in the device 200. The upper casing 201 comprises the detector arrangement 205 for detecting reaction light from the reaction vessel 110. The detector arrangement 205 may comprise any suitable components such as a photodiode for example. The lower casing 202 is configured to receive the reaction vessel 110 on the reaction vessel holder 120. The reaction vessel holder 120 is coupled to a thermal device 130 for heating and/or cooling the reaction vessel holder 120. The lower casing further comprises an excitation arrangement 203 for transmitting an excitation beam through the reaction vessel holder 120 to the reaction vessel placed on top of the reaction vessel holder 120. A guide arrangement comprising one or more mirrors 204 is provided in the lower casing to steer the excitation beam from the excitation source 203 toward the reaction vessel 110. A member 114, in the form of a clamp, is provided to secure the reaction vessel holder to the thermal device 130.

In this embodiment, the reaction vessel holder 120 is configured to be transmissive for wavelengths of the excitation beam.

In this device, the thermal device is a single thermoelectric cooling unit, and the reaction vessel holder can be thermally cycled up to about 20° C. per second.

Other components may be present in the device, such as filters and imaging or focusing lenses. The device additionally comprises a control system that is described with reference to FIG. 4, which is configured accordingly for single sample detection.

Device for Molecule Detection in a Plurality of Reaction Chambers

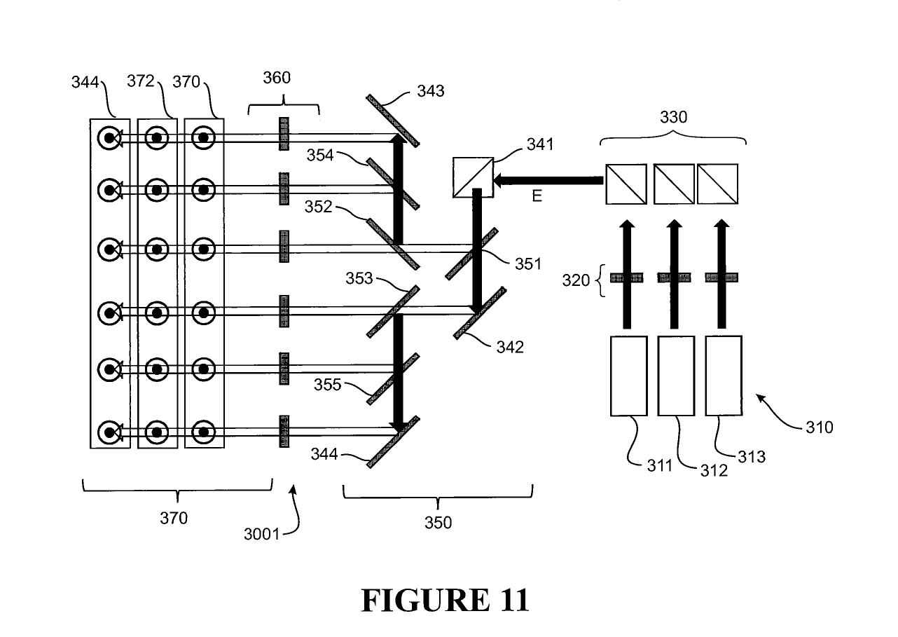

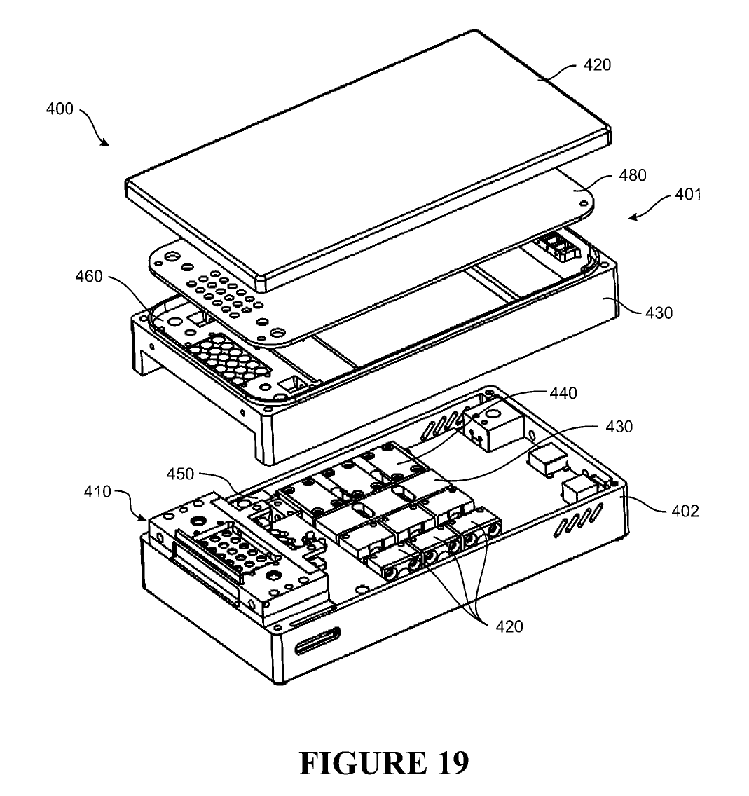

According to other embodiments of the present invention, the device can be used for detection of molecule(s) within a plurality of reaction chambers. Implementation of such a device will be discussed in detail below with reference to FIGS. 11 to 34.

Referring to FIG. 11, the device comprises an excitation arrangement 310. The excitation arrangement comprises three excitation sources 311-313, and each excitation source is configured to transmit an excitation beam at a different wavelength in the interval of about 400 nm to about 800 nm for example. The excitation arrangement 310 comprises a first excitation source 311 for transmitting an excitation beam at a red wavelength, a second excitation source 312 for transmitting an excitation beam at a green wavelength, and a third excitation source 313 for transmitting an excitation beam at a blue wavelength. Each of the first, second and third excitation sources 311-313 comprises a Nichia laser diode or any other suitable diode from other manufacturers. The laser diode is configured to emit an excitation beam at the suitable wavelength. According to an alternative embodiment, the excitation arrangement 310 may comprise a single excitation source for transmitting an excitation beam at a single wavelength.

The device further comprises a collimator for collimating the excitation beam from the excitation sources. The collimator may comprise a collimating lens and may be integral with or separate from the excitation sources. Referring to FIG. 11, the collimator is integral with the excitation sources 311-313. According to other embodiments, the collimator is separate from the excitation sources 311-313.

The collimated excitation beams are then adapted to pass through an attenuator and a wavelength filter. As shown in FIG. 11, the attenuator and wavelength filter are shown be part of a single components 320. According to other embodiments of the device, the attenuator and the wavelength filter are separate components, and the position of the attenuator and the position of the wavelength filter are interchangeable with each other.

The attenuator is for reducing the power of the excitation beam. The attenuator comprises a neutral density (ND) filter. In a further embodiment, the attenuator reduces the power of the excitation beam by a factor of about 10. In a further embodiment, the attenuator is positioned between the collimator and the beam splitter arrangement. In a further embodiment, about 100 mW is incident on the ND filter, and about 10 mW exits the ND filter to the beam splitter arrangement. In an alternative embodiment, the optical assembly may comprise at least one attenuator for reducing the power of at least one of the plurality of split excitation beams from the beam splitter arrangement.

The wavelength filter is for filtering any spectral components in the excitation beam from the collimator that fall within a band of the reaction light from the sample in at least one of the reaction vessels. In a further embodiment, the wavelength filter comprises a laser diode clean-up filter. In a further embodiment, the wavelength filter is adapted to block reaction light wavelengths in the excitation beam, to prevent interference of the excitation beam and the reaction light. In an additional or alternative embodiment, the device may comprise at least one wavelength filter, the or each wavelength filter for filtering spectral components in at least one of the plurality of split excitation beams from a beam splitter arrangement that will be described in further detail below.

The device comprises beam combination optics 330 for combining the three excitation beams from the different excitation sources 311-313 into a single excitation beam E. In one embodiment, the beam combination optics comprises two interference filters for combining the excitation beams. In another embodiment, the beam combination optics comprises one interference filter and one polarizing cube beam splitter/combiner. A beam steering mirror is used to steer the excitation light E from the excitation sources toward a beam splitter arrangement 3001.

Apertures are found throughout the device. For example, the apertures may take the form of the various apertures in the various beam splitters through which the excitation beam propagates.

The excitation beam E then passes through a beam splitter arrangement 3001 for splitting the excitation beam into a plurality of split excitation beams.

The beam splitter arrangement 3001 has one or more beam splitters, depending on the number of reaction vessel chambers that the device is configured for. The beam splitter arrangement 3001 is configured to split the excitation beam E from the excitation source into a plurality of split excitation beams. The or each beam splitter is configured to split an incoming beam into two beams. Where the beam splitter arrangement 3001 comprises more than one beam splitter, the beam splitters are arranged in tiers such that a first tier comprises one beam splitter for receiving the excitation beam from the collimator, and each of the other tiers comprises one or more beam splitters, each beam splitter in at least one of the other tiers being configured to receive a split excitation beam from a previous tier. In some embodiments, components of the beam splitter arrangement are arranged to output evenly spaced split excitation beams. In further embodiments, components of the beam splitter arrangement have a generally planar arrangement, with the components being arranged in a common plane. In some of these further embodiments, the direction of split excitation beams exiting the beam splitter arrangement is orthogonal to the common plane.

A beam splitter is defined as an optical element that receives one input beam and ‘splits’ it to two split beams. One beam splitter cannot produce more than two beams from a single incoming beam without additional optical elements (mirrors, corner cubes and other reflective elements). There exist other optical elements (dispersive elements such as prism and diffraction grating) which can split a single monochromatic beam into a plurality of beams (zeroth, first, second order and so on). These are dispersive elements and not beam splitters in the context of the specification. A grating is not suitable for the preferred embodiment devices of the present invention for spatial and intensity purposes (the zeroth, first, second, third and higher order beams all have different power from a grating). In some embodiments, the beam splitter may be configured to split selected beams but not others. For example, the beam splitter may be configured to split beams at the excitation wavelength(s) and allow beams at other wavelengths to pass.

The beam splitters of the beam splitter arrangement 3001 may together be a single monolithic optical component. In a monolithic optical assembly, an optical index matching material can be used to fill interstitial air gaps and fuse together adjacent beam splitters which are in optical communication. In practice, the monolithic assembly is formed from pieces of optical material of the required geometry (for example trapezoidal and/or right angle parts).

The beam splitter may be a cube beam splitter or a plate beam splitter. A cube beam splitter is configured to receive a single beam or a plurality of spaced apart beams, and to split the or each beam into two split beams, each split beam having substantially the same or different intensities. In the described embodiments, a cube beam splitter typically receives a single beam and splits the beam into two split beams. A plate beam splitter is configured to receive one beam or a plurality of spaced apart beams, and to split the or each beam into two split beams, each split beam having substantially the same or different intensities. Where a beam splitter arrangement comprises more than one beam splitter, the beam splitters may be plate beam splitters, cube beam splitters, or a combination of plate and cube beam splitters. In addition, where a beam splitter arrangement comprises a plurality of beam splitters, a cube or plate beam splitters could be used to replace two or more beam splitters in the beam splitter arrangement. For example, where a tier of a beam splitter arrangement comprises four beam splitters, two, three, or all of the beam splitters in that tier could be replaced by a single cube or plate beam splitter.

The cube beam splitter arrangement may comprise a polarising beam splitter or a non-polarising beam splitter. Where the arrangement comprises a polarising beam splitter, the excitation beam entering the polarising beam splitter may first be passed through a half wave plate, before being incident onto the polarising cube beam splitter. A polarising cube beam splitter is capable of splitting the excitation beam equally into two split excitation beams. Alternatively, a half wave plate may not be provided, and the polarising beam splitter or the excitation source may be rotated accordingly such that the excitation beam to the polarising beam splitter is split into two beams of substantially equal or unequal intensities. The non-polarising beam splitters can be designed to be wavelength and polarisation independent to produce to split beams of substantially equal intensities (to within about 5%). The beam splitter arrangement 3001 may comprise a combination of at least one half-wave plate, at least one polarising cube beam splitter, and at least one non-polarising cube beam splitter.

Several different configurations of the beam splitter arrangement 3001 are possible. In the different configurations of the beam splitter arrangement, the split excitation beams that exit the beam splitter arrangement are preferably substantially parallel and/or substantially orthogonal to each other. This enables a particularly compact optical arrangement and thereby a compact overall device.

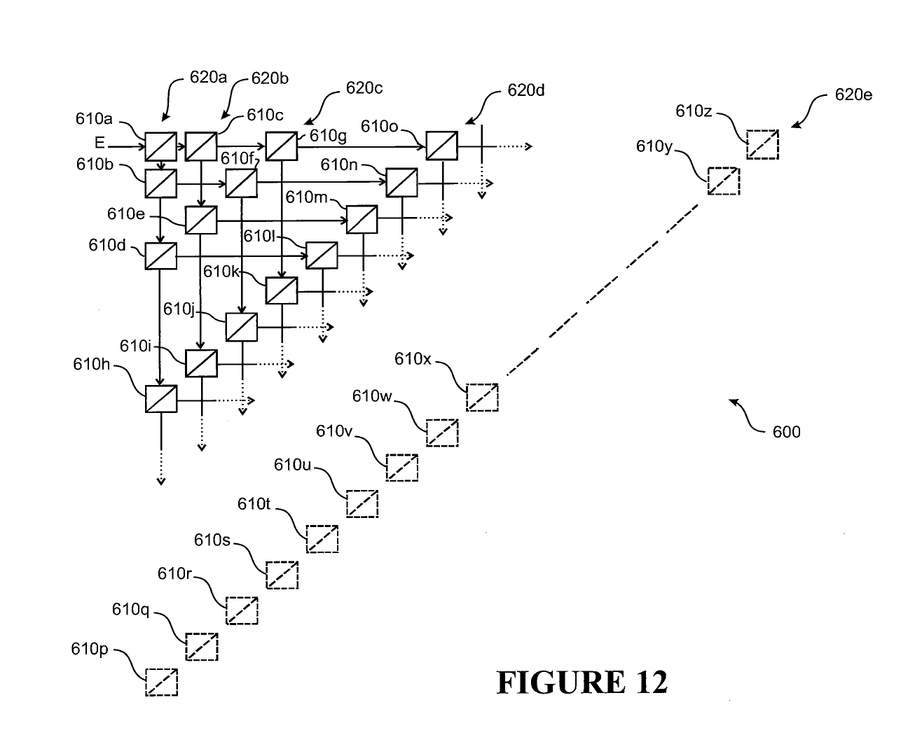

By way of first example, with reference to FIG. 12, the beam splitter arrangement 600 comprises 2ⁿ−1 number of beam splitters 610a-z configured to split the excitation beam E into 2ⁿ number of split excitation beams of substantially equal intensity and wavelength, n being an integer greater than zero. The or each beam splitter 610a-z is configured to split an incoming beam into two beams. In the case where n is more than one, the beam splitters 610a-z are arranged in n number of tiers 620a-e such that a first tier 620a contains one beam splitter 610a for receiving the excitation beam and an i^{th} tier contains 2ⁱ⁻¹ beam splitters, i being an integer ranging from 2 up to n. The or each respective beam splitter in a tier is associated with two respective beam splitters in a next tier such that two beams split by a respective beam splitter in a tier are split further into four beams by the associated beam splitters in the next tier. The second tier 620b comprises two beam splitters 610b-c for receiving the split excitation beams from the beam splitter 610a in the first tier 620a. The third tier 620c comprises four beam splitters 610d-g for receiving the split excitation beams from the second tier 620b. The fourth tier 620d comprises eight beam splitters 610h-o for receiving the split excitation beams from the third tier 620c. The n^{th} tier 620e comprise 2ⁿ⁻¹ beam splitters 610p-z for receiving the split excitation beams from the (n−1)^{th} tier.

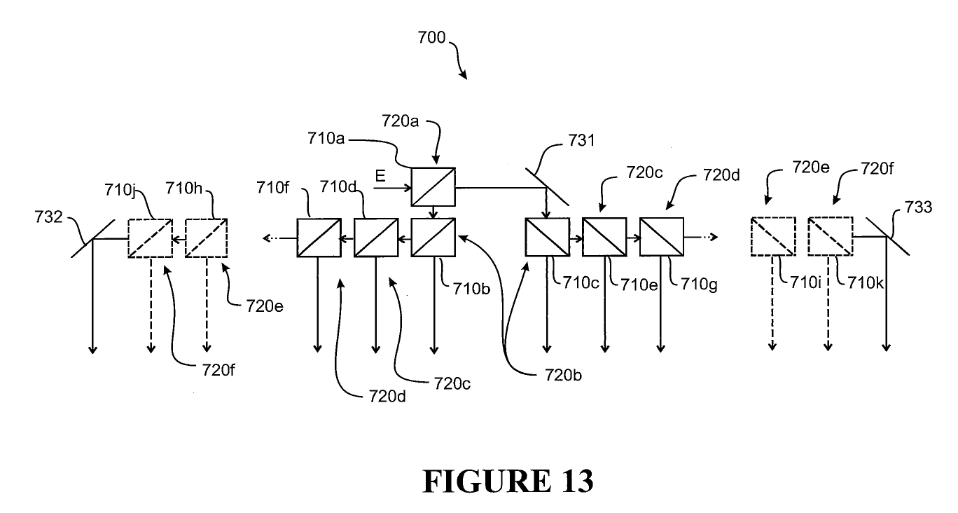

By way of second example, with reference to FIG. 13, the beam splitter arrangement 700 is configured to split the excitation beam E into up to k number of split excitation beams, k being an even integer greater than two, wherein the beam splitters 710a-k are arranged in m number of tiers 720a-f, where m is an integer greater than 1 and k=2×m. A first tier 720a contains one beam splitter 710a that is configured to receive the excitation beam, and to split the incoming beam into two split excitation beams. An i^{th} tier, i being an integer ranging from 2 to m, is configured to receive incoming beams from a previous tier and to split each incoming beam into two split excitation beams, wherein in the case where i is less than m, one of the split excitation beams is directed to the next tier and the other split excitation beam is one of the k split excitation beams, and in the case where i equals m, the m^{th} tier is each split excitation beams from the m^{th} tier is one of the k split excitation beams. The second tier 720b comprises two beam splitters 710b-c and is configured to receive two of the four split excitation beams from the first tier 720a. The third tier 720c comprises two beam splitters 710d-e and is configured to receive two of the four split excitation beams from the second tier 720b. The fourth tier 720d comprises two beam splitters 710f-g and is configured to receive two of the four split excitation beam from the third tier 720c. An (m−1)^{th} tier comprises two beam splitters 710h-i and is configured to receive two of the four split excitation beams from the (m−2)^{th} tier. An m^{th} tier 720f comprises two beam splitters 710j-k and is configured to receive two of the four split excitation beams from the (m−1)^{th} tier 720e. Optical steering mirrors 731-733 are provided to allow for a more compact arrangement of the components of the beam splitter arrangement.

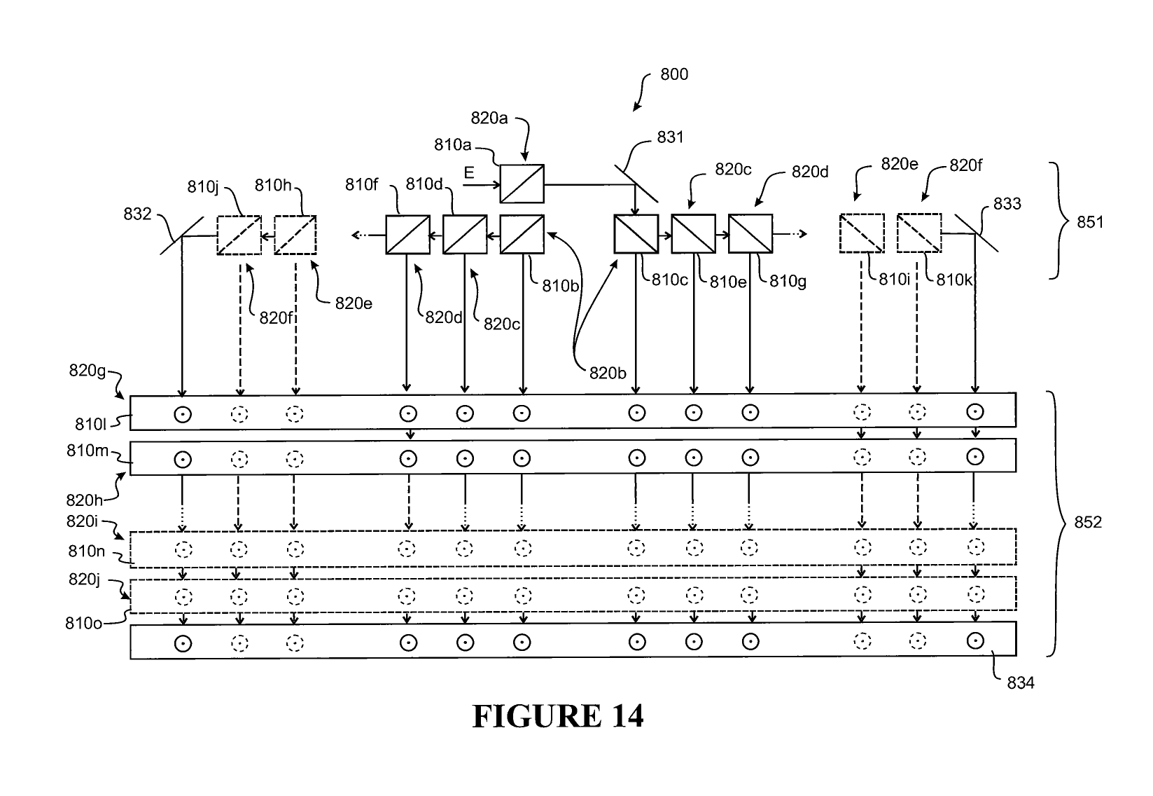

By way of third example, with reference to FIG. 14, the beam splitter arrangement 800 is configured to split the excitation beam E into up to k number of split excitation beams, k being an even integer greater than two, wherein the beam splitters 810a-810o are arranged in (m+n) number of tiers, where m and n are integers indicating the number of primary tiers 851 and secondary tiers 852 respectively, m being greater than one and n being greater than zero, and k=2×m×(n+1), such that the first tier 820a, which is one of the primary tiers 851, contains one beam splitter 810a that is configured to receive the excitation beam, and to split the incoming beam into two split excitation beams. An i^{th} tier, which is one of the primary tiers 851, i being an integer ranging from 2 to m, is configured to receive incoming beams from a previous tier and to split each incoming beam into two split excitation beams, wherein in the case where i is less than m, one of the split excitation beams is directed to the next tier and the other split excitation beam is directed to the (m+1)^{th} tier, and in the case where i equals m, the split excitation beams from the m^{th} tier are directed to the (m+1)^{th} tier of the secondary tiers. A second tier 820b comprises two beam splitters 810b-c and is configured to receive the split excitation beams from the first tier 820a. A third tier 820c comprises two beam splitters 810d-e and is configured to receive two of the four split excitation beams from the second tier 820b. A fourth tier 820d comprises two beam splitters 810b-c and is configured to receive two of the four split excitation beams from the third tier 820b. A (m−1)^{th} tier 820e comprises two beam splitters 810h-i and is configured to receive two of the four split excitation beams from the (m−2)^{th} tier. An m^{th} tier 820f comprises two beam splitters 810b-c and is configured to receive two of the four split excitation beams from the (m−1)^{th} tier 820e. A p^{th} tier, which is one of the secondary tiers 852, j being an integer ranging from m+1 to m+n, is configured to receive incoming beams from a previous tier and to split each incoming beam into two split excitation beams, wherein in the case where j is less than m+n, one of the split excitation beams is directed to the next tier and the other split excitation beam is one of the k split excitation beams, and in the case where j equals m+n, each split excitation beam from the (m+n)^{th} tier is one of the k split excitation beams. An (m+1)^{th} tier 820g comprises a beam splitter 8101 and is configured to receive the split excitation beams from the previous tiers 820b-f. An (m+2)^{th} tier 820h comprises a beam splitter 810m, and is configured to receive the split excitation beams from the previous tier 820g. An (m+n−1)^{th} tier 820i comprises a beam splitter 810n, and is configured to receive the split excitation beams from the previous tier. An (m+n)^{th} tier 820j comprises a beam splitter 810o, and is configured to receive the split excitation beams from the previous tier 820i. Optical steering mirrors 731-733 are provided to allow for a more compact arrangement of the components of the beam splitter arrangement.

The embodiment described with reference to FIGS. 11 and 17 to 23 relates to the beam splitter arrangement of the third example, which is configured accordingly for the reaction vessel 110′ with eighteen reaction chambers, as described with reference to FIG. 3.

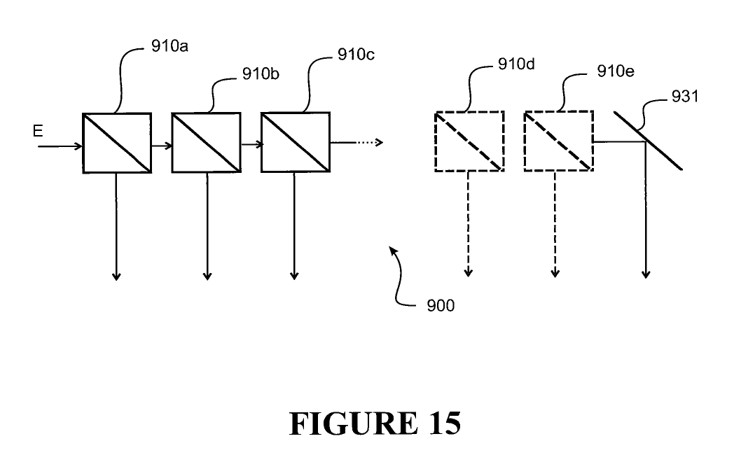

By way of fourth example, with reference to FIG. 15, the beam splitter arrangement 900 is configured to split the excitation beam E into up to k number of split excitation beams, k being an integer greater than two, wherein the beam splitters 910a-e are arranged in m number of tiers, where m is an integer greater than one and k=(m+1). An i^{th} tier, i being an integer ranging from 1 to m, is configured to split the excitation beam from the collimator. In the case where i is less than m, the i^{th} tier is configured to receive an incoming beam from the collimator if i equals 1, or from a previous tier if i is more than 1, and to split the incoming beam into two split excitation beams, one of the split excitation beams is directed to the next tier and the other split excitation beam is one of the k split excitation beams. In the case where i equals m, the m^{th} tier is configured to receive and split an incoming beam from a previous tier, and each split excitation beam from the m^{th} tier is one of the k split excitation beams. An optical steering mirror 931 is provided to allow for a more compact arrangement of the components of the beam splitter arrangement 900.

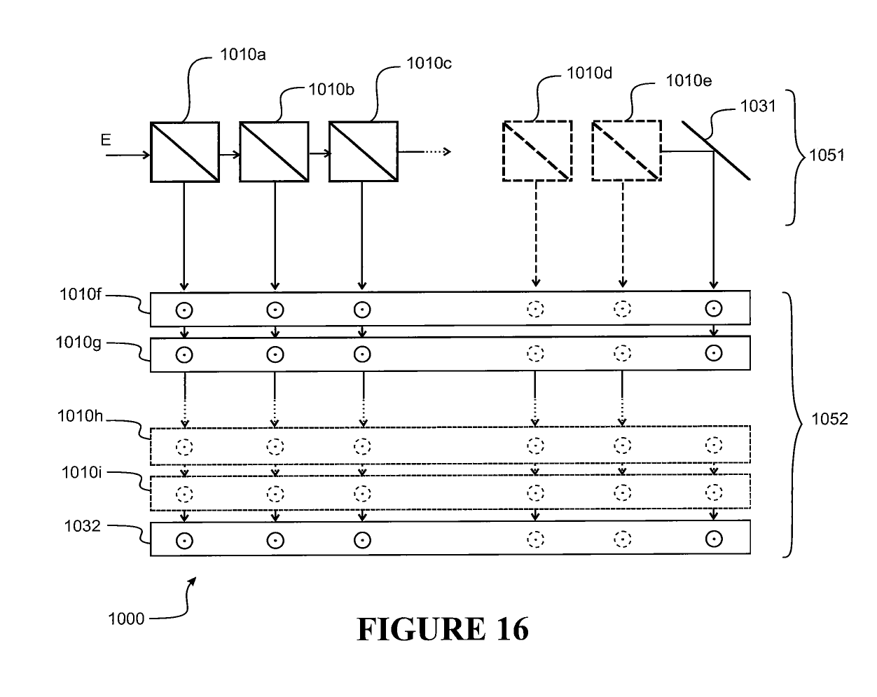

By way of fifth example, with reference to FIG. 16, the beam splitter arrangement 1000 is configured to split the excitation beam into up to k number of split excitation beams, k being an even integer greater than two. The beam splitters are arranged in (m+n) number of tiers, where m and n are integers indicating the number of primary tiers 1051 and secondary tiers 1052 respectively, m being greater than one and n being greater than zero, and k=(m+1)×(n+1). An i^{th} tier, which is one of the primary tiers 1051, i being an integer ranging from 1 to m, is configured to split the excitation beam from the collimator. In the case where i is less than m, the i^{th} tier is configured to receive an incoming beam from the collimator if i equals 1, or from a previous tier if i is more than 1, and to split the incoming beam into two split excitation beams, one of the split excitation beams is directed to the next tier and the other split excitation beam is directed to the (m+1)^{th} tier. In the case where i equals m, the m^{th} tier is configured to receive and split an incoming beam from a previous tier and to direct the split excitation beams to the (m+1)^{th} tier of the secondary tiers. A j^{th} tier, which is one of the secondary tiers 1052, j being an integer ranging from m+1 to m+n, is configured to receive incoming beams from a previous tier and to split each incoming beam into two split excitation beams. In the case where j is less than m+n, one of the split excitation beams is directed to the next tier and the other split excitation beam is one of the k split excitation beams. In the case where j equals m+n, each split excitation beam from the (m+n)^{th} tier is one of the k split excitation beams.

Referring back to FIG. 11, the beam splitter arrangement of the device for detecting molecule(s) in eighteen reaction vessel chambers has five tiers, three of which are primary tiers 350 and two of which are secondary tiers 370. A first tier, which is one of the primary tiers 350, comprises one beam splitter 351 and is configured to receive the excitation beam from the excitation source 310 and to split the excitation beam into two beams of substantially equal intensities. A second tier, which is one of the primary tiers 350, comprises two beam splitters 352, 353. The second tier is configured to receive two incoming beams from the beam splitter 351 in first tier and to split each incoming beam into a split excitation beam of about 33% intensity and a split excitation beam of about 67% intensity. A third tier, which is one of the primary tiers 350, comprises two beam splitters 354, 355. The third tier is configured to receive the two split excitation beams of about 67% intensity from the beam splitters 352, 353 of the second tier and to split each incoming beam into two split excitation beams of substantially equal intensities. A fourth tier, which is one of the secondary tiers 370, comprises a plate beam splitter 371. The fourth tier is configured to receive the two 33% intensity split excitation beams from the beam splitters 352, 353 of the second tier and four split excitation beams from the beam splitters 354, 355 of the third tier and to split each incoming beam into a split excitation beam of about 33% intensity and a split excitation beam of about 67% intensity. A fifth tier, which is one of the secondary tiers 370, comprises a plate beam splitter 372. The beam splitter 372 of the fifth tier is configured to receive the six split excitation beams of about 67% intensity from the beam splitter 371 fourth tier and to split each incoming beam into two split excitation beams of substantially equal intensities. The eighteen split excitation beams of substantially equal intensity and wavelength comprise six split excitation beams of about 33% intensity from the beam splitter 371 of the fourth tier and twelve split excitation beams from the beam splitter 372 of fifth tier. The split excitation beams from the secondary tiers and mirror are in a direction out of the page.

Attenuators 360 are provided between the primary tiers 350 and the secondary tiers 370. The attenuators 360 attenuate the power of the split excitation beams from the primary tiers 350 accordingly such that the six beams from the primary tiers 360 are of substantially equal power. The attenuator may for example comprise a neutral density (ND) filter or linear polarizer.

The beam splitter arrangement may comprise a polarising cube beam splitter or non-polarising cube beam splitters. Where the beam splitter arrangement comprises a polarising cube beam splitter, the excitation beam entering the polarising cube beam splitter is first passed through a half wave plate, before being incident onto the polarising cube beam splitter. Alternatively, a half wave plate may not be provided, and the polarising beam splitter or the excitation source may be rotated accordingly such that the polarising beam splitter can produce to split beams of substantially equal or unequal intensities. The beam splitter arrangement may comprise a combination of at least one half-wave plate, at least one polarising cube beam splitter, and at least one non-polarising cube beam splitter.

The primary tiers 350 and secondary tiers 370 both comprise plate beam splitters. According to other embodiments, the primary tiers 350 may comprise cube beam splitters, while the secondary tiers 370 may comprise plate beam splitters. According to an alternative embodiment, the primary tiers 350 and secondary tiers 370 may both comprise cube beam splitters.

According to an alternative embodiment of the beam splitter arrangement, the beam splitters of the primary tiers are together a primary monolithic optical component, and the beam splitters of the secondary tiers are together a secondary monolithic optical component. The primary monolithic optical component forms five beam splitters and three mirrors, and is configured to receive a single excitation beam and to output six split excitation beams according to the primary tiers of the third example beam splitter arrangement. Alternatively, the primary monolithic optical component forms five beam splitters and one mirror, and it configured to receive a single excitation beam and to output six split excitation beams according to the primary tiers of the fifth example beam splitter arrangement. The secondary monolithic optical component forms two beam splitters and a mirror and is configured to receive the six split excitation beams output by the primary monolithic optical component, and to output eighteen split excitation beams. Optical index matching material is used to fill interstitial air gaps and to fuse together adjacent beam splitters. The monolithic optical components can be formed from a plurality of pieces of optical material of the required geometry (for example trapezoidal and/or right angle parts).

The device comprises mirrors 341-344 for folding the optical path of the excitation beams accordingly to provide a compact arrangement of the device.

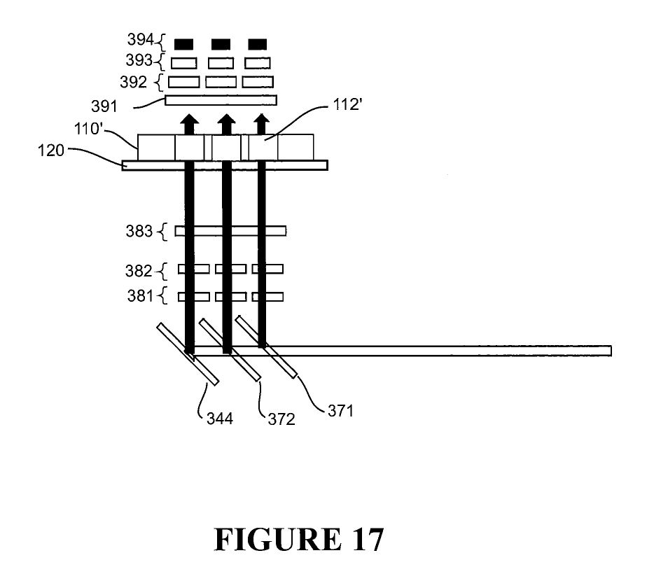

FIG. 17 shows the arrangement for guiding three of the eighteen excitation beams from the beam splitter arrangement shown in FIG. 11 to the reaction vessel 110′ and for guiding the reaction light from the reaction vessel 110′ to the detector arrangement 394. Each excitation beam from the beam splitter 371 of the fourth tier, the beam splitter 372 of the fifth tier and the mirror 344 are respectively guided through an attenuator 381 followed by a polariser 382.

The attenuators 381 attenuate the power of the split excitation beams from the secondary tiers 370 accordingly such that the eighteen beams from the secondary tiers 370 are of substantially equal power. The attenuator may for example comprise a neutral density (ND) filter.

The polarisers 382 for each channel, attenuates the portion of incident light that is not aligned with the optical axis of the polarising element. The polarisers 382 have a substantially circular circumference. As the laser diode light is at all times linearly polarised throughout the optical assembly, the linear polarisers 382 offer the means to precisely trim the laser power (through rotating the polariser). Laser power is trimmed to better than 1% in this way. The importance of trimming the laser power is two-fold. Firstly, the fluorescent signal from the samples is proportional (outside of saturation effects) to the incident laser power. Widely differing signals from identical samples is not desirable. Secondly, photo bleaching effects of the sample (if present) will vary among otherwise identical samples due to variation in incident laser power thereby compromising the quality of the gathered data further.

The device comprises a plurality of guide arrangements. Each guide arrangement is configured to guide a respective one of the plurality of split excitation beams along an excitation path from the beam splitter arrangement into a reaction vessel containing a sample to stimulate an emission of a reaction light from the sample. Each guide arrangement is further configured to guide reaction light from the sample along a detection path towards a detector.

Each beam from the polarisers passes through a filter element 383, which is configured to pass the excitation beam while reflecting any reaction light. The filter element 383 is part of the guide arrangement. Alternatively, the filter element 383 may be configured to block or attenuate the reaction light. The filter element 383 guides the excitation beam to the reaction vessel 110′ to stimulate an emission of reaction light from the sample in the reaction vessel 110′. In an alternative embodiment, the filter element 383 may not be present. In that embodiment, a bottom surface of the reaction vessel holder 120 may be coated with an optical coating for passing excitation beams, while reflecting reaction light.

The filter element 383 is a suitable multi-transition interference filter element, such as a trichroic element, a notch filter, or a multi-bandpass filter for example to allow the different wavelengths of the excitation arrangement to pass through to the reaction vessel 110′. An example of a suitable multi-transition interference filter may for example be a BrightLine® triple-band bandpass filter from Semrock. The filter element may alternatively comprise an arrangement of dichroic (‘two colour’) elements. Alternatively, where a single excitation wavelength is used, the filter element 383 may be a dichroic element.

In another embodiment each excitation beam may additionally be passed through a focusing lens to focus the excitation light into the sample. The focusing lenses may be located either side of the filter element 383.

Each of the plurality of split excitation beams passes into a respective one of the reaction vessel chambers. The excitation beam passes through a reaction vessel holder 120 that is described with reference to FIG. 4 into a reaction vessel 110′ that is described with reference to FIG. 3. The excitation light stimulates an emission of reaction light from the sample in the reaction vessel 110′.

Reaction light from the reaction vessel 110′ passes through a filter element 391, which is configured to block or reflect the excitation beams, while passing the reaction light towards the detector assembly. The filter element 391 is part of the guide arrangement.

The reaction light passes through imaging/focusing lenses for focusing the reaction light onto the detector arrangement 394. Similar to the filter element 383 for the excitation beams, the filter element 391 for the reaction light allows different wavelengths of the reaction light to pass through to the detector 394. The filter element 391 may comprise an arrangement of dichroic (‘two colour’) elements. Alternatively, where a reaction wavelength is emitted from the sample in the reaction vessel 110, the filter element 391 may be a dichroic element. Alternatively the filter element 391 may be a coloured glass absorption filter with dichroic coating applied to it. Alternatively the filter element 391 may comprise a dichroic filter and a separate coloured glass absorption filter.

The reaction light passes through imaging/focusing lenses 392 to focus/image the reaction light onto the detector arrangement 394.

The reaction light passes through glass filters 393, which act as an additional block to excitation beams. The glass filters 393 may be the same type of filter as the first filter element 391. Alternatively, the imaging/focusing lenses may be positioned immediately after the reaction vessel holder, before the second dichroic element.

The detector arrangement 394 comprises a silicon photodetector. The photodetector is configured to generate an electrical photocurrent proportional to incident light intensity (for further amplification via electrical means). The photodetector may be for example an FDS100 PIN photodiode provided by Thorlabs of Newton, N.J.

The optical transparency of the reaction vessel holder 120 that can be heated and/or cooled accordingly allows for the excitation arrangement and the detector arrangement to be positioned facing opposite sides of the reaction vessel in a ‘sandwich’ topology. This ‘sandwich’ topology allows for an extremely compact device construction.

In an alternative configuration of the device, the device can be configured in an upward looking geometry to transmit excitation beams to the reaction vessel through the reaction vessel holder, and to receive reaction light from the reaction vessel through the reaction vessel holder. In this form, the output from the primary tiers of beam splitters into the secondary tiers of beam splitters would be similar to that previously described. The beam splitters of the secondary tiers would be transparent at the reaction light wavelength to receive reaction light through the reaction vessel holder.

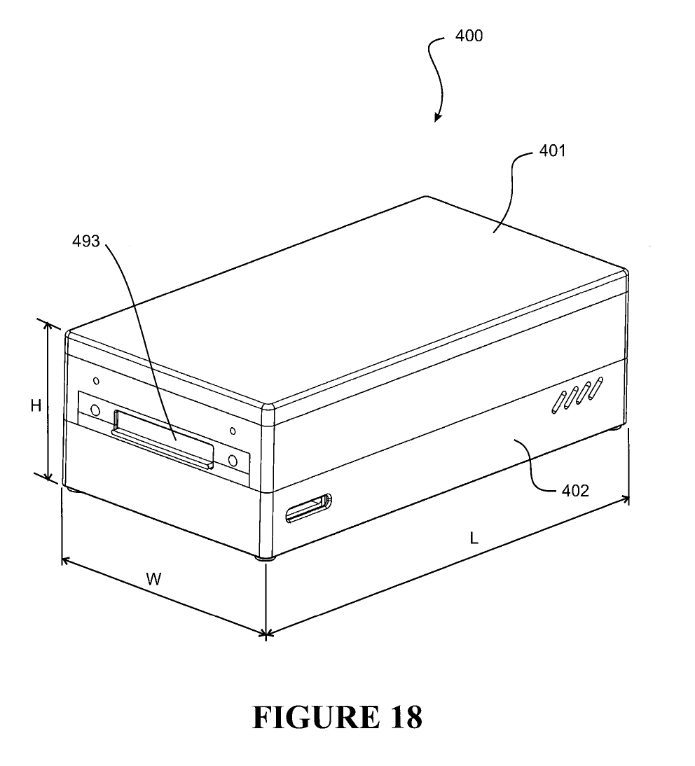

A first example hand-holdable casing of the device for molecule detection in eighteen reaction chambers will now be described with reference to FIGS. 18 to 23. Optical components are not shown in these figures, but will be described with reference to FIGS. 11 and 17.

The device 400 comprises an upper casing 401 and a lower casing 402. The reaction vessels 110 are removably insertable into the lower casing 402. The lower casing 402 comprises a door 493 that can be opened to receive the reaction vessel 110. The door 493 may be a sliding door for example. Alternatively, the upper casing 401 is moveable relative to the lower casing 402 between a closed configuration and an open configuration.

The upper casing 401 houses the controller, battery, power supply, and the optical assembly for detection of the reaction light. The lower casing 402 houses the excitation source, a power jack, a USB interface hub, a power switch, the beam splitter arrangement and the reaction vessels. The upper casing 401 comprises a cap 420 which is removably connected to the upper casing 401. The upper casing 401 further comprises an reaction light optical housing 460 for housing components for detecting the reaction light such as imaging/focusing lenses 392 and the glass filters 393. The upper casing 401 further houses the main controller board 440, which comprises the array of photodetectors 394 for reaction light detection. In an alternative configuration, the photodiodes may be mounted in a separate housing from the controller board 480.

The lower casing 402 comprises an optical assembly housing 410, which will be described in further detail below. The lower casing further comprises the excitation arrangement housing 420 for housing the three excitation sources 311-313, the attenuator and wavelength filter housing 430 for housing the attenuator and wavelength filter components 320 and the beam combination optics housing 440 for housing the beam combination optics 330. A primary tiers housing 450 is provided for housing components of the primary tiers 350 of the beam splitter arrangement. The lower casing 402 is preferably metallic.

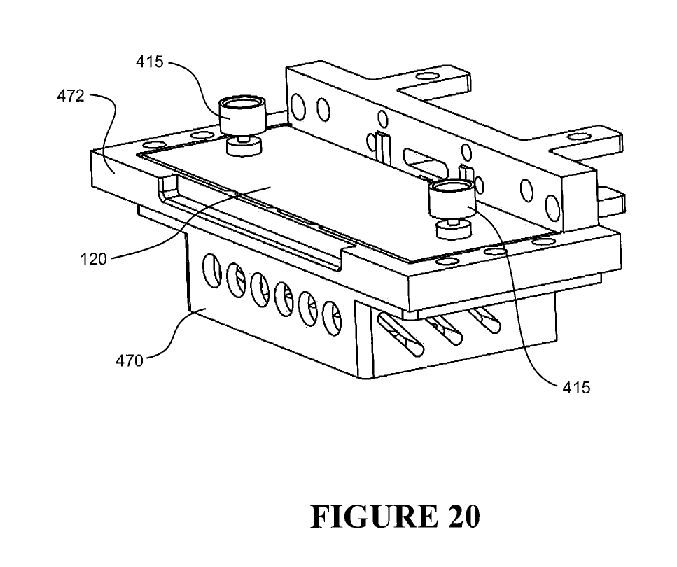

The optical assembly housing 410 is configured to receive the reaction vessel 110′ and for housing the reaction vessel holder and optical components for transmitting the excitation beams to the reaction vessel 110. Referring to FIG. 20, the optical assembly housing 410 (with the reaction light guide housing for the optical components for guiding the reaction light removed) comprises a secondary tiers housing 470 for housing beam splitters of the secondary tiers 370. The optical assembly 410 further comprises a thermal device housing 472 for housing the thermal device and the reaction vessel holder 120. Features of the reaction vessel holder 120 have been described previously. The thermal device is configured to heat and/or cool the reaction vessel holder accordingly. The optical assembly housing 410 further comprises clamps 415 for securing the reaction vessel holder in the direction normal (vertical) to its extended surface. The reaction vessel holder is constrained in the horizontal plane along its edges by small contact points protruding from the thermal device housing 472.

FIG. 21 shows an exploded view of the optical assembly 410. The reaction light guide housing 490 is configured to guide reaction light from the reaction vessel 110′ to the detector arrangement in the upper housing 401. The reaction light guide housing 490 is configured to receive the filter element 391 for the reaction light. The reaction light guide housing comprises the door 493 that has been previously described.

In an embodiment, a reaction vessel securing member in the form of vertical detent pins 416, are provided to removably secure the reaction vessel to the reaction vessel holder. The reaction vessel securing member is configured to apply a downward force onto the reaction vessel when the reaction vessel is placed on top of the reaction vessel holder for increasing the physical and thermal contact between the reaction vessel and the body. The vertical detent pins 416 are provided with ball bearing ends, which press vertically into matching recesses in the reaction vessel 110′. The detent pins 416 horizontally and vertically secure reaction vessel 110′. The reaction vessel 110′ is provided with apertures 115′ that respectively engage a respective one of the detent pins 416. Alternative reaction vessel securing member(s) could be used.

The clamps 415 are configured to vertically secure the reaction vessel holder 120 to the thermal device, which are two thermoelectric cooling units 414. With two thermoelectric cooling units 414, the reaction vessel holder 120 can be thermally cycled at up to about 40° C. per second. Each clamp 415 is respectively positioned directly above a respective one of the thermoelectric cooling units 414.

The thermal device housing 472 comprises two apertures 471, and each aperture 471 is configured to receive a respective one of the thermoelectric cooling devices 414. As described above, the thermal device housing 472 is configured to receive the reaction vessel holder 120 and to constrain movement of the reaction vessel holder 120 in the horizontal plane. The thermal device housing 472 further comprises an ejector mechanism 476 for ejecting the reaction vessel 110′ from the device through the door 493. The ejector mechanism 476 is coupled to an actuator 477 that is actuatable by a user to cause the ejector mechanism to eject the reaction vessel 110′ from the device.

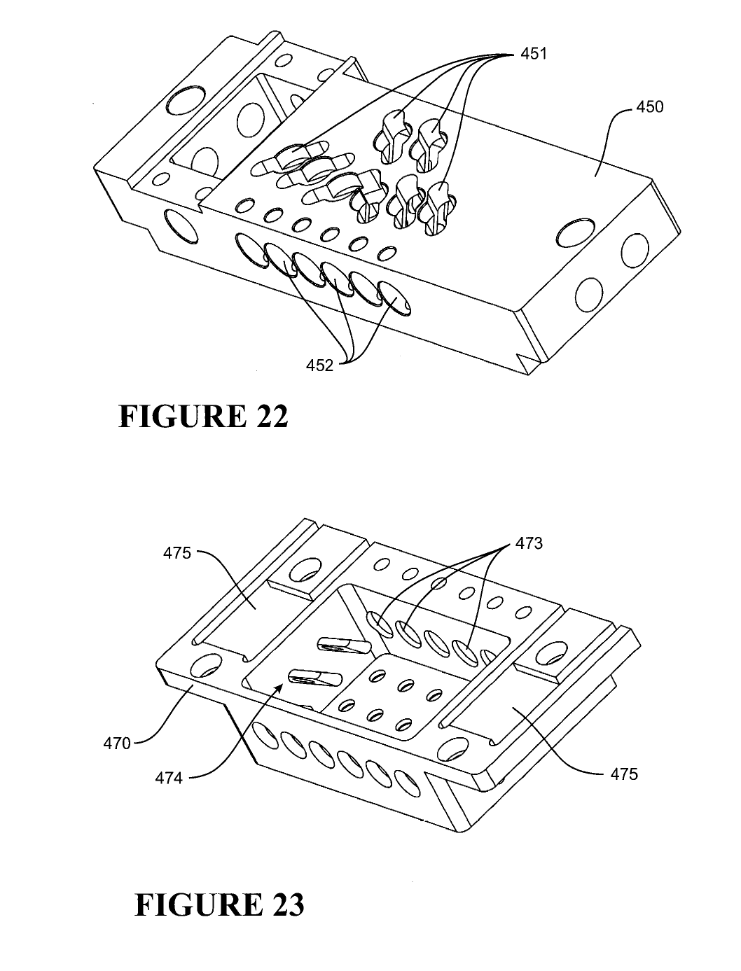

Referring to FIG. 22, the primary tiers housing 450 is for housing the primary tiers 350 of the beam splitter arrangement. The primary tiers housing 450 is block-shaped having a plurality of slots 451, wherein each slot is configured to receive a respective one of the beam splitters 351-355 or a respective one of the steering mirrors 342-344. In an alternative embodiment, the primary tiers housing 450 does not comprise slots, and may instead comprise a recess for receiving the beam splitters 351-355 and mirrors 342-344. The housing 450 has a series of apertures and channels 452 to allow the excitation beam to be split and pass to the next tier or to the secondary tiers 370 in the secondary tiers housing 470. The primary tiers housing 450 is located adjacent to the secondary tiers housing 470 as shown in FIG. 16.

Referring to FIG. 23, the secondary tiers housing 470 for housing the secondary tiers 370 of the beam splitter arrangement comprises a series of apertures 473 for receiving the excitation beam from the primary tiers housing 450. The secondary tiers housing 470 further comprises a recess 474 for receiving the beam splitters 371, 372 of the secondary tiers 370 or the mirror 344. The reaction vessel holder 120 is positioned substantially above the recess 474, and the split excitation beams are directed upwards from the recess 474. The secondary tiers housing 470 further comprises two recesses 475. The cold side of the thermoelectric cooling units 414 is located in the recess 475. The secondary tiers housing 470 is substantially metallic and in thermal communication with the metallic lower casing 402 of the device, which acts as the heatsink for the thermoelectric cooling units 414.

Referring back to FIG. 18, in one embodiment, the dimensions of the device are (length×width×height) 120 mm×64 mm×42 mm. The dimensions of the upper casing 70″ are L=120 mm×W=64 mm×H=21 mm, and the dimensions of the lower casing 80″ are L=120 mm×W=60 mm×H=16 mm.

Preferably, the dimensions of the casings of the devices of the present invention are such that each device is capable of being held in the palm of a single adult hand. The devices preferably have dimensions of between about (length×width×height) 110 mm×54 mm×32 mm and about 220 mm×120 mm×105 mm. The optical head of each device where beam splitting and guiding takes place is substantially smaller than the overall casing itself. By way of example, the optical head and supporting structures for a four channel device may have dimensions of approximately 40 mm×50 mm×60 mm, with the additional space in the upper cavity being used by a battery and power supply. This means that the casing dimensions could be reduced further with advances in battery technology.

The device additionally comprises a control system that is described with reference to FIG. 4, which is configured accordingly for molecule detection in eighteen reaction vessel chambers.

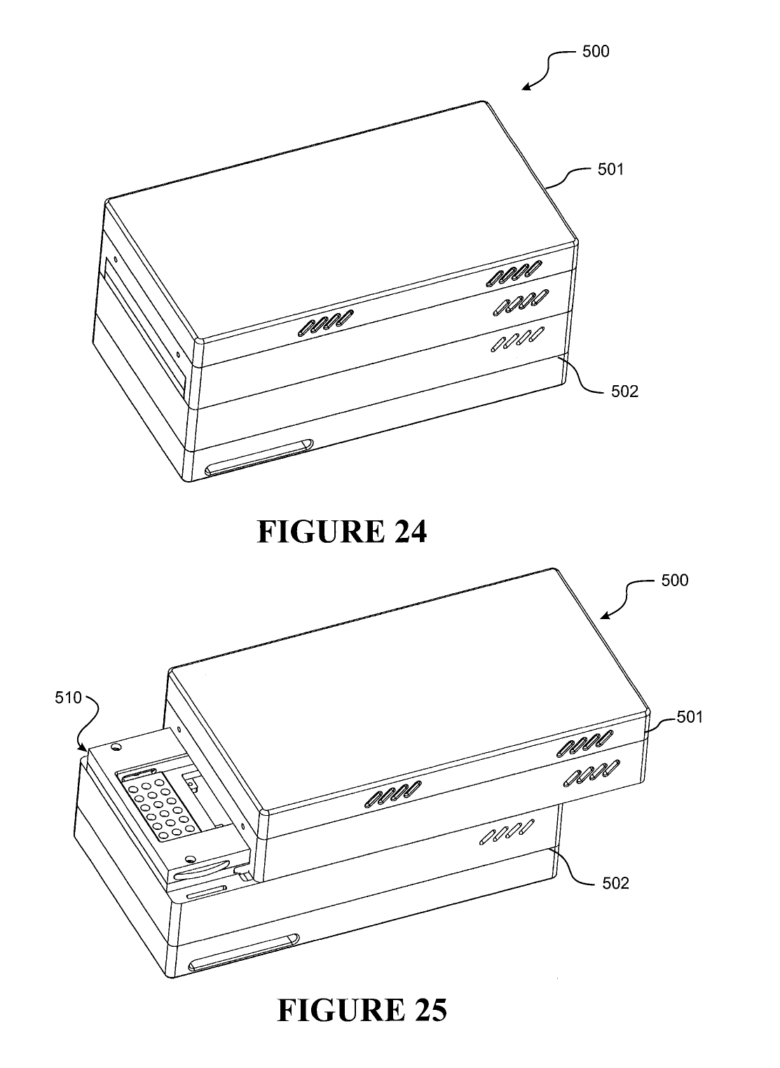

A second example casing of the hand-holdable device 500 for molecule detection in eighteen reaction chambers will now be described with reference to FIGS. 24 to 34. In the following description of the alternative construction of the device casing 500, parts having like reference numerals as those used to describe the first example casing 400 with the addition of 100 indicate like parts. Unless stated otherwise, these like parts operate substantially in a similar manner to that described above.

The device 500 comprises an upper casing 501 and a lower casing 502. The lower casing 502 is slidable relative to the upper casing 501 between a closed configuration (shown in FIG. 24) and an open configuration (shown in FIG. 25). When the lower casing is in the open configuration, the reaction vessel can be positioned within or removed from the lower casing 502. The lower casing 502 is configured to receive the optical assembly housing 510. The housing 510 is configured to house the reaction vessel 110′, the reaction vessel holder 120, components of the beam splitter arrangement, and components of the guide arrangement.

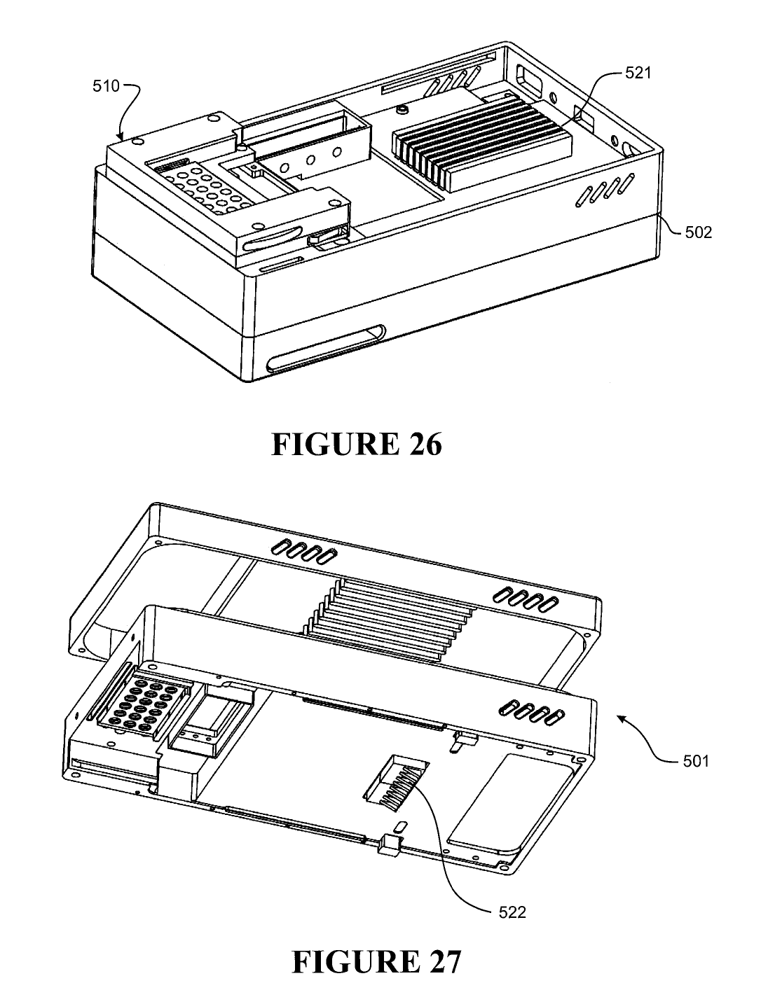

To facilitate an electrical connection between components in the upper casing 501 and the lower casing 502, the upper casing 501 and lower casing 502 are provided with sliding interconnects (shown in FIGS. 26 and 27). Electrically conductive teeth or contacts 522 (seen in FIG. 27) in the upper casing 501 are slidable along electrically conductive tracks 521 (seen in FIG. 26) in the lower casing 502. According to other embodiments of the device, the teeth may be provided in the lower casing, and the tracks may be provided in the upper casing. The teeth 522 and tracks 521 may comprise any suitable electrically conductive material to facilitate the electrical connection therebetween. For example, the teeth may be gold coated.

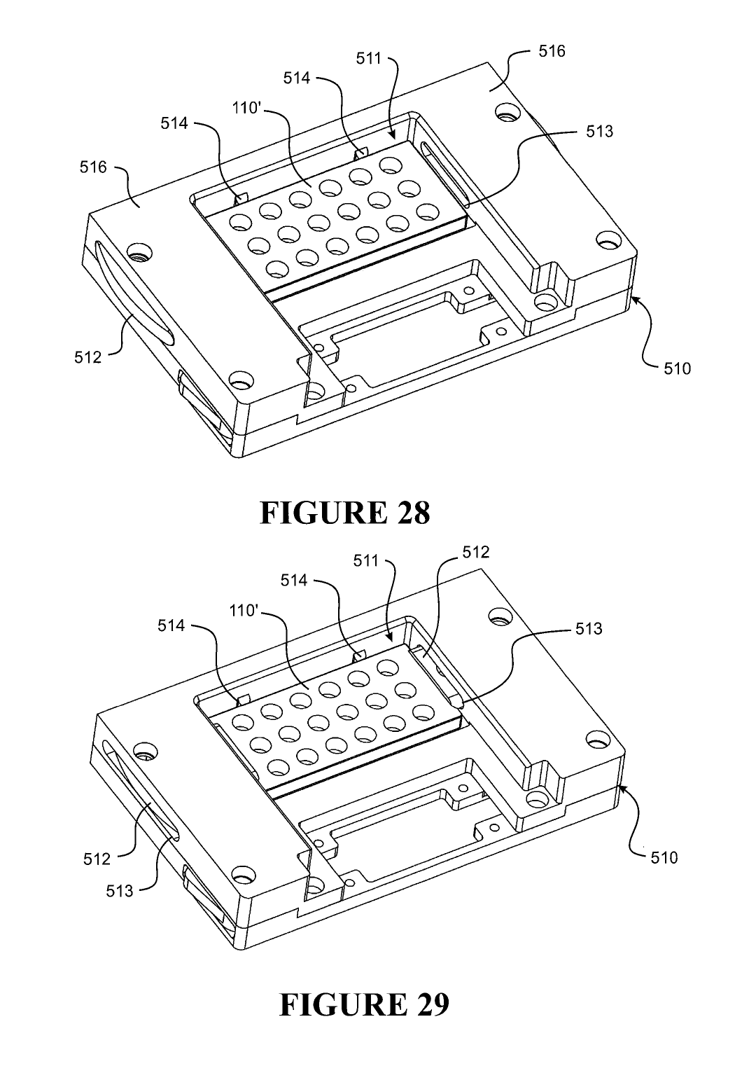

FIGS. 28 and 29 show the optical assembly housing 510 and reaction vessel securing members 512 with a reaction vessel 110′. The optical assembly housing 510 is configured to receive the reaction vessel 110′ in a receiving area 511 between the reaction vessel securing members. The optical assembly housing 510 comprises two reaction vessel securing members positioned on opposite sides or ends of the optical assembly housing 510.

The securing members are in the form of spring-loaded disks 512. When the device 500 is in the open configuration, the spring loaded side disks 512 are in a release configuration as shown in FIG. 28. In this configuration, the disks 512 are in their outer position away from the receiving area 511 and do not hold the reaction vessel 110′.

When the device is in the closed configuration, the spring loaded side disks 512 are in a secure configuration as shown in FIG. 29. In this configuration, the disks 512 are in their inner position and extend within the receiving area 511 to clamp the reaction vessel 110′ in a substantially central position in the receiving area 511 firmly against the reaction vessel holder 120. The side disks 512 are compressed inward by the inner walls of the upper casing 501 of the device 500 as the upper casing 501 closes. According to a preferred embodiment, the disks 512 are biased to the released configuration. When the upper casing 501 is slid from the closed configuration to the open configuration, the disks 512 move outwards into the released configuration under spring tension. According to other embodiments, the disks may be manually operated between the secure configuration and release configuration.

The optical assembly housing 510 comprises sides 516 with two apertures 513 through which the disks 512 can move between the secure and release configurations. Referring to FIG. 28, when the device 500 is in the open configuration, the disks 512 are in the release configuration and are biased outwardly away from the receiving area 511. In this configuration, the outer edge of each disk 512 protrudes outside the respective side 516 through the aperture 513. Referring to FIG. 29, when the device 500 is in the closed configuration, the internal walls of the upper casing 501 push the disks 512 through the respective apertures 513 inwards towards the receiving area 511 to secure the reaction vessel 110′ to the reaction vessel holder 120, by pushing against the outer edges of the securing members 512 through the respective apertures 513 in the sides 516. The optical assembly housing 510 has stoppers 514 for limiting the movement of the reaction vessel 110′.

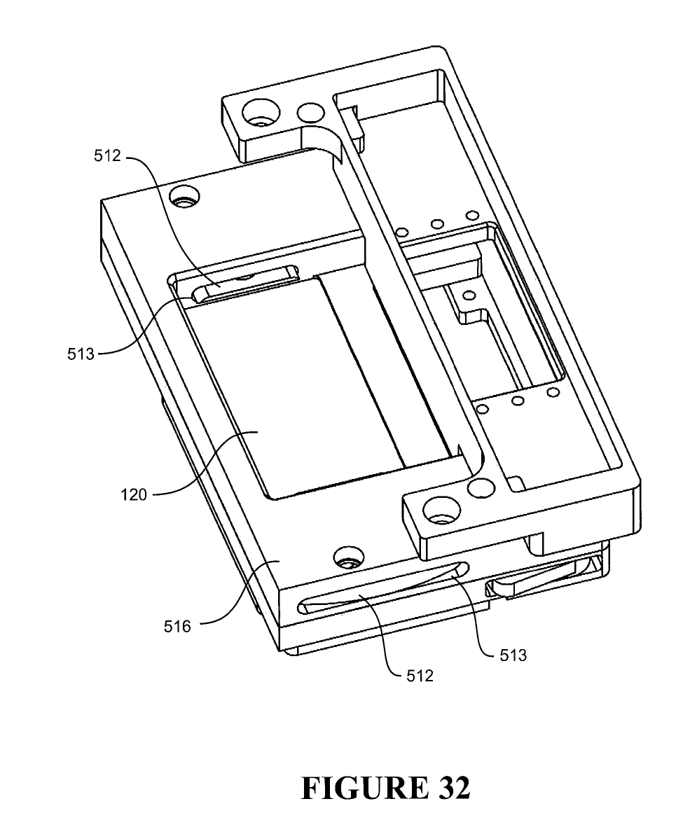

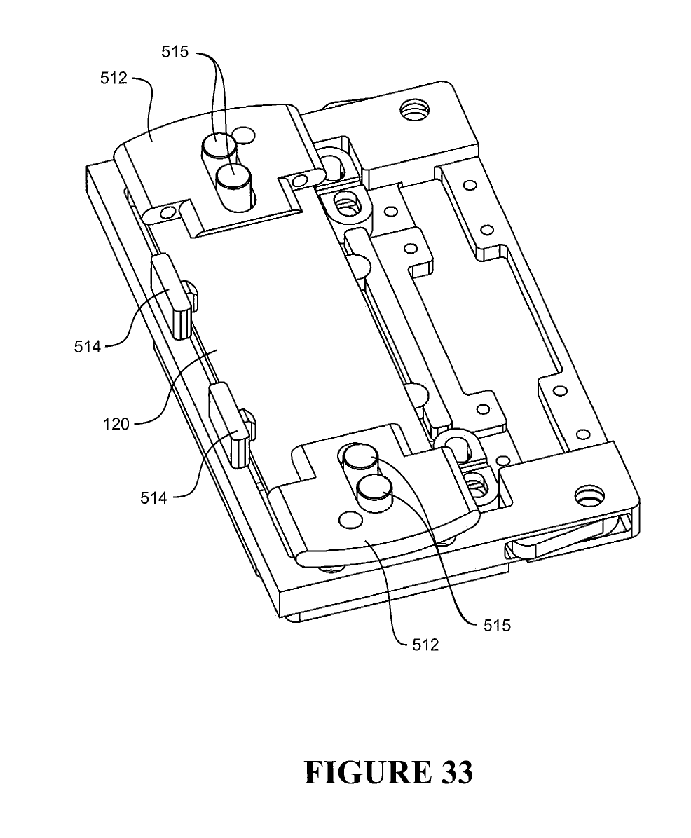

The stoppers 514 restrict or inhibit any movement of the reaction vessel 110′ when the device is in the closed configuration. The stoppers 514 can also prevent the section of diamond plate 130 below the stoppers 514 from rising vertically when the stoppers 514 rest on the diamond plate. Vertical retention of the diamond plate is achieved primarily with the members 515, which are described below with reference to FIG. 33.

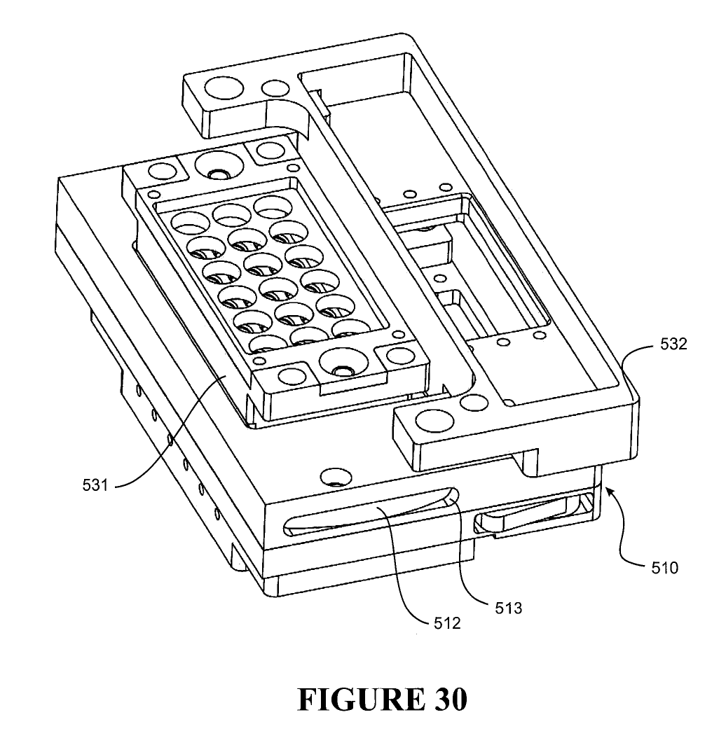

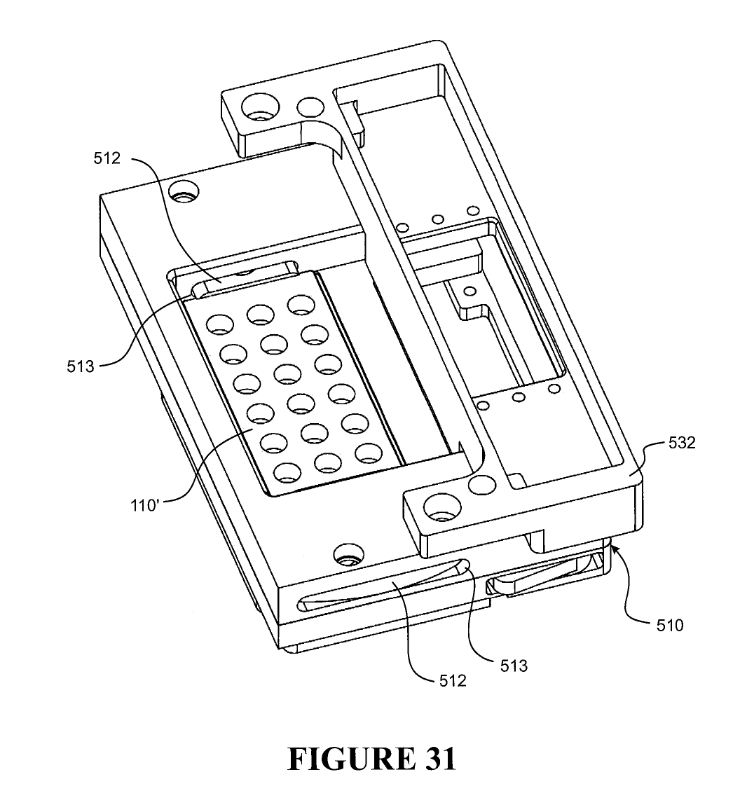

FIG. 30 shows an internal view of the some components of the device 500 in the closed configuration. In this configuration, the guide housing 531 that houses the optical components of the guide arrangement is positioned directly above the reaction vessel 110′. The guide housing 531 is part of the upper casing 501. FIG. 31 shows the internal view of the device without the guide housing 531. The component 532 in the upper casing 501 secures the reaction vessel holder 110′ towards the stoppers 514 in the closed configuration of the device 500.

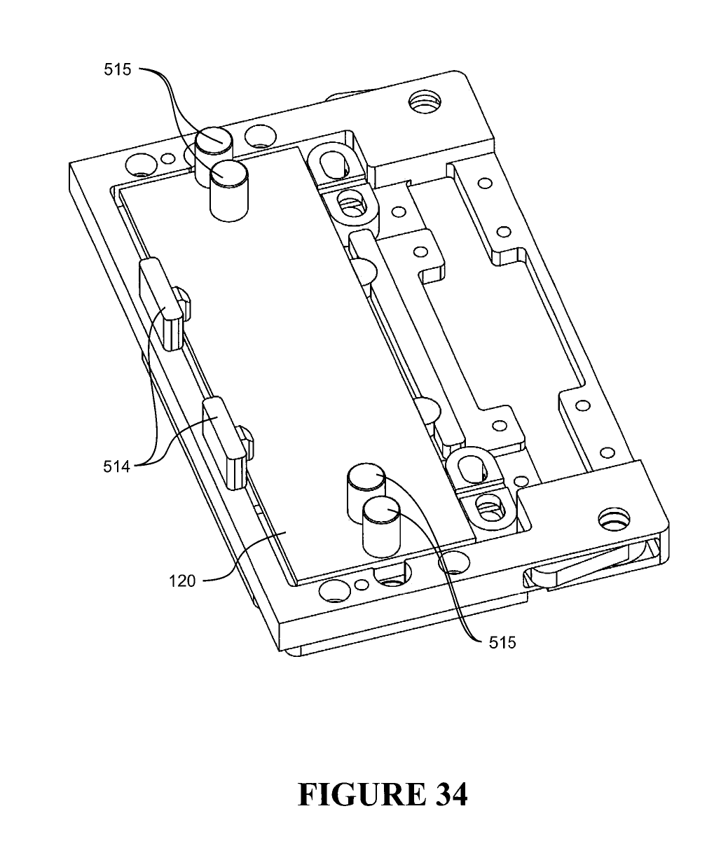

FIG. 32 shows the internal view of the device 500 without the reaction vessel 110′. FIG. 33 shows the internal view of the device with casing part 516 removed, which reveals the members 515 that secure the reaction vessel holder 120 to the thermal devices 512. The disks 512 are slidable between the release configuration and the secure configuration along the members 515. The members 515 are retained in place by the removed casing part 516. These members 515 are spring loaded and push down on the reaction vessel holder 120 via a small spring. FIG. 34 shows the members 515 and stoppers 514 for securing the reaction vessel holder 120.

The optical layout of the device of the present invention allows for a tight ‘sandwich’ layout of the reaction vessel holder, the reaction vessel and the following filters and detectors. For example, a distance between the reaction vessel holder and the detector may be up to about 20 mm, and could be as low as about 10 mm. Preferably, the distance between the reaction vessel holder and the detector is between about 10 mm and 15 mm. The optical layout of the device of embodiments of the present invention described herein uses normal incidence optics and filters in the reaction light detection assembly. According to embodiments of the device of the present invention, in the case where no fluorescence reflective coating is present on the reaction vessel, on the reaction vessel holder or the filter intermediate the reaction vessel holder and the excitation assembly arrangement, while the reaction light from the sample propagates in all directions, only reaction light that propagates in the same direction as the excitation light, towards the detector arrangement, is collected. However, the tight proximity of the reaction light imaging/focusing lens with the reaction vessel means the solid angle subtended from the sample to the lens is approximately 0.8 steradians.

Experiment 1—Use of Single Sample Hand Held Molecule Detection Device for Quantitative PCR

Introduction

The performance of a preferred embodiment single channel hand held device (1sHHD), as described above with reference to FIGS. 7-10, as a quantitative Polymerase Chain Reaction (Q-PCR) instrument was measured against a standard laboratory-based Q-PCR instrument supplied commercially from Roche. This commercial system was the Roche LightCycler 480 (LC480).

The Q-PCR assay used for this work amplified the Jellyfish Green Fluorescent Protein (GFP) sequence encoded in cloning vector eGFP-N1 from CLONTECH. Q-PCR reactions were run in parallel on both the 1sHHD and Roche LC480 instruments. Comparison between the technologies was facilitated through graphing fluorescence against cycle number, cycle threshold (Ct: the cycle number at which the measured fluorescence crossed a set threshold) and agarose gel electrophoresis. All reactions shared the same Q-PCR assay components and used the same sealing foil and thermal-cycling conditions. Each reaction was set up from the same master mix and was carried out on both instruments at the same time.

Methods

Instrumentation:

Two devices were used in the tests outlined below. These were the Q-PCR Roche LightCycler 480 (LC480) and the single sample hand held device (1sHHD) as described above with reference to FIGS. 7-10. The reaction vessel holder used in the device comprises a synthetic diamond plate.

Source of Q-PCR Template:

Genomic DNA (gDNA) from a GFP transgenic mouse was used to generate template for this work. The gDNA was extracted from 22 mg of GFP transgenic mouse liver using the ZyGem prepGEM Tissue kit as per the manufacturer's instructions. This gave a transgenic gDNA sample with a concentration of 20 ng/μl.

The CLONTECH eGFP-N1 vector was used to construct the GFP transgenic mouse. PCR primers were designed to this vector to amplify the GFP DNA encoding sequence. The NCBI primer design tool, available on the NCBI website (http://ncbi.nlm.nih.gov) was used to design the forward and reverse primers for GFP. Primers are given in Table 1.

TABLE 1
Q-PCR Primers

| Primer Name | Sequence |
|---|---|
| eGFP-Short | TTCAGCCGCTACCCCGACCA (SEQ ID NO: 1) |
| Forward | |
| eGFP-Short | CGGTTCACCAGGGTGTCGCC (SEQ ID NO: 2) |
| Reverse | |

The 20 μl Q-PCR reaction used contained the following mix of components: 10 μl SYBR Green I Master Mix (Roche, Cat No. 04 707 516001); 1 μl (20 pmol) of both Forward and Reverse Primer; 7 μl PCR quality H₂O; and 1 μl of a 10⁻⁶ dilution of the transgenic gDNA sample. The GFP amplicon was generated by PCR on the GeneAmp 9700 (Applied Biosystems) instrument using thermal cycle conditions 95° C. for five minutes followed by 40 cycles of 95° C. for 30 seconds, 60° C. for 30 seconds and 72° C. for 30 seconds. These three temperatures constitute a cycle: DNA denaturation at 95° C., primer annealing at 60° C. and primer extension at 72° C. The amplicon was checked by agarose gel electrophoresis (data not shown) to confirm production of the correct sized product. This amplicon was diluted by 10⁻² to give the Initial DNA Template for the experiments described here.

Q-PCR Method:

Each Q-PCR reaction mix contained 10 μl SYBR Green I Master Mix (Roche, Cat No. 04 707 516001); 1 μl (20 pmol) of both Forward and Reverse Primer; 7 μl PCR quality H₂O; and 1 μl of DNA or H₂O. A Master Mix of Q-PCR reagents consisting of enough reagents to perform multiple reactions was set up for each experiment. To do this, the volumes required for each component making up a 20 μl reaction were multiplied by the number of reactions needed for the experiment (in this case 12) plus one extra to allow for pipetting errors (13).

Q-PCR reactions carried out using the LC480 used multiwell plate plasticware and sealing foils (Cat No. 04 729 692 001). Q-PCR reactions performed on the 1sHHD were undertaken in custom-made clear acrylic plastic disc chambers which were sealed with Roche sealing foils (Cat No. 04 729 757 001).

Identical thermal-cycle conditions were used on both devices to amplify the target GFP sequence from template. These conditions were 95° C. for 45 seconds followed by 40 cycles of 95° C. for 10 seconds, 60° C. for 10 seconds and 72° C. for 10 seconds. These three temperatures constitute a cycle: DNA denaturation at 95° C., primer annealing at 60° C. and primer extension and fluorescence measurement at 72° C.

Results

A master mix for twelve reactions was divided into six tubes. 1 μl of a 10⁻³ dilution of the Initial DNA Template was added to three of these tubes. These tubes were labelled DNA 1, DNA 2 and DNA 3. Two microlitres of the reaction mix was removed from each of the three DNA tubes and stored on ice. These were ‘before amplification’ controls for reactions that had DNA added to them. Tubes that had no DNA added were labelled Background 1, Background 2 and Background 3. This resulted in six sub-master mixes to allow for paired Q-PCR runs; one for the LC480 and the other for the 1sHHD.

Six clear plastic clear acrylic disc chambers were set up for the 1sHHD. The reaction vessels are similar to the reaction vessel that is described with reference to FIGS. 1 and 2 To ensure that reactions performed on the two instruments were as identical as possible, 40 μl of each sub-master mix was loaded on the disc chamber. The reaction mix was pipetted up-and-down at least 10 times before 20 μl was removed to the LC480 plate. The remaining 20 μl was sealed into the disc chamber using Roche sealing foil ensuring no bubbles were trapped between the foil and the solution.

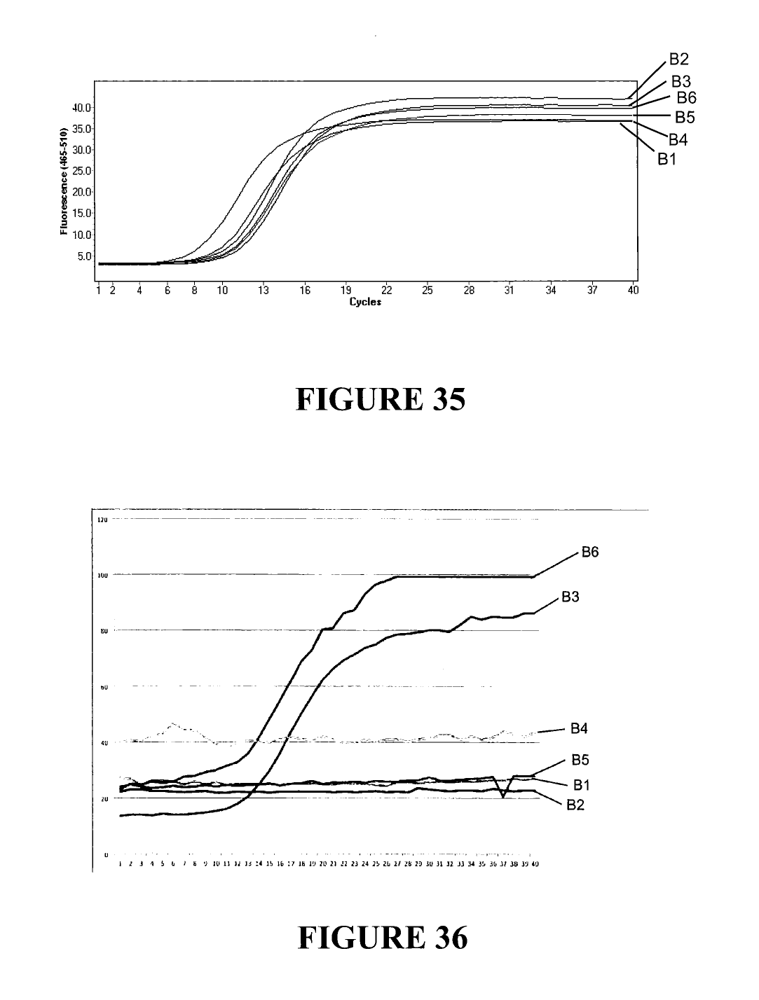

Amplification profiles from the LC480 and 1sHHD are given in FIGS. 35 and 36 respectively.

FIG. 35 shows LC480 quantitative DNA amplification curves generated from six reaction mixes directly corresponding to samples amplified on the 1sHHD. FIG. 36 shows the 1sHHD quantitative DNA amplification curves generated from six reaction mixes directly corresponding to samples amplified on the Roche LC480. The curves in these figures correspond to Background 1(curve B1), Background 2 (curve B2), Background 3 (curve B3), DNA 1 (curve B4), DNA 2 (curve B5) and DNA 3 (curve B6).

It is evident from the LC480 that every reaction mix contained enough GFP template to generate a classic sigmodial amplification curve during the Q-PCR process. However, for 1sHHD amplified samples only two reaction mixes produced classic sigmodial amplification curves. Curves B3 and B6 were produced from sample Background 3 and DNA 3. DNA 3 was the first chamber run on the 1sHHD for this series of experiments. DNA3 had not been stored on ice while waiting for a turn on the 1sHHD. For Background 3, a drop of a thermal coupling oil such as Immersion Oil (Olympus Optical Co. LTD Immersion Oil 8CC) was added to the 1sHHD diamond thermal plate prior to loading the disc chamber before commencing the amplification cycle.

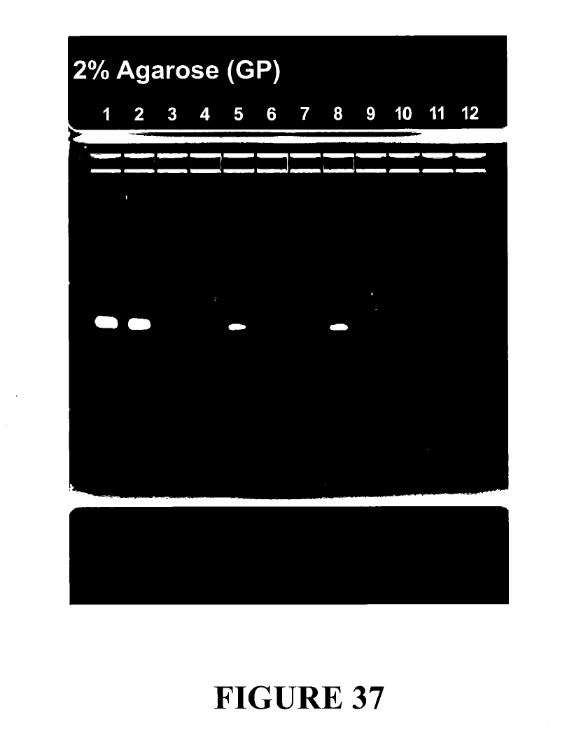

These observations suggested that, for the 1sHHD, DNA amplification had not occurred in those reactions where a sigmodial curve had not been produced. The reasoning was that, while on ice, primer/template complexing had occurred. Unlike the case of the LC480, the transfer of heat to the disc chamber was not sufficient to break up the complexed aggregates, thus preventing amplification of the template. Adding a thermal coupling oil between the disc chamber and the diamond plate facilitated heat transfer which allowed for aggregate breakdown and DNA amplification to commence. To test this hypothesis 2 μl aliquots of each reaction before and after amplification were tested on an agarose gel (E-Gel 2% Agarose GP, Life Sciences Cat No. G501802). Only those 1sHHD reactions which showed Q-PCR amplification curves contained DNA molecules of the expected size. DNA was not detected in reaction mixes prior to the PCR amplification process, as shown in FIG. 37.

FIG. 37 shows agarose gel electrophoresis of 2 μl of each reaction mix before and after Q-PCR amplification. The twelve lane contents are 1) Background 3 following LC480 amplification; 2) DNA 3 following LC480 amplification; 3) Background 1 after 1sHHD amplification; 4) Background 2 after 1sHHD amplification; 5) Background 3 after 1sHHD amplification; 6) DNA 1 after 1sHHD amplification; 7) DNA 2 after 1sHHD amplification; 8) DNA 3 after 1sHHD amplification; 9) DNA 1 before 1sHHD amplification; 10) DNA 2 before 1sHHD amplification; 11) DNA 3 before 1sHHD amplification; 12) Q-PCR mastermix, no amplification and no DNA.

A comparison of the Ct values for samples that successfully amplified on the 1sHHD to those amplified on the LC480 shows similar levels of detection sensitivity. If the threshold is set so that it shows the point at which the amplification curve first lifts away from background, the LC480 measurements for samples DNA 3 and Background 3 would give a Ct of approximately 8. For the same 1sHHD amplified samples Cts are approximately 8 and 9 respectively. This would suggest that the sensitivity of the 1sHHD is the same or similar to the LC480.

Conclusion

The 1sHHD successfully performs Q-PCR to the same or similar sensitivity as the Roche LC480. These functions include both detection of amplified product for end-point evaluation methods and quantitative analysis. These results indicate that the 1sHHD and other devices containing this technology can be used in Q-PCR systems.

Experiment 2—Use of Single Sample Hand Held Molecule Detection Device as a Fluorometer to Estimate DNA Concentration

Introduction

The 1sHHD was investigated as a fluorometer to estimate DNA concentration. Knowing the concentration of nucleic acid is an important part of molecular biology practice. Whether it is undertaking PCR, cloning, sequencing or library construction, knowing the concentration of the nucleic acid sample under investigation is the first step in these procedures. Traditionally, nucleic acid concentration is determined by spectrophotometry. Nucleic acid maximally absorbs light at 260 nm wavelength. By measuring the absorbance of a solution of nucleic acid at 260 nm, the amount of material present can be calculated using a constant value, dependent on the type of nucleic acid, of X μg/μl absorbs at 10D unit at 260 nm. For DNA, this constant is 50 μg/μl.

Recently, alternative, more sensitive methods for determining nucleic acid concentration have been used. Specifically, these methods are based on fluorometry. In this technique, an intercollating dye that changes its fluorescent characteristics when bound to nucleic acid is employed. The dye binds to the material present in the sample, the dye is excited and emissions within the reporting spectrum measured. Concentration is determined by comparison to a standard curve generated from solutions of known nucleic acid concentration.

In this experiment, the preferred embodiment single sample hand held device (1sHHD), as described above with reference to FIGS. 7-10, was used as a fluorometer. This experiment further demonstrates the importance of controlling sample temperature.

Methods

Instrumentation

The single sample hand held device (1sHHD), that is described with reference to FIGS. 7-10, was used in this work. Spectrophotometer measurements were undertaken using the MBA 2000 (Perkin-Elmer, USA). The reaction vessel holder used in the device comprises a diamond plate.

Sample Preparation

All DNA samples were diluted in molecular biology grade water (5Prime Ref no. 2500010). Standards were generated using the DNA molecular weight marker III (Roche Cat no. 10 528 552001) supplied at a concentration of 0.25 μg/μl. For fluorometric measurements, 10 μl of sample or standard was added to 10 μl of SYBR Green I Master Mix (Roche Cat no. 04 707 516001). For spectrophotometric measurements, the DNA standard or sample was added directly to the measuring cuvette and the absorbance measured.

Program to Measure Fluorescence on 1sHHD

To measure fluorescence, the 1sHHD was programmed to equilibrate the sample for 10 seconds at 25° C. and then to perform 3 cycles holding at 25° C., 60° C. and 95° C. as set out in Table 2. This ensured six measurements at temperatures 25° C. and 60° C. and three at 95° C. for each sample or standard. Fluorescence units were expressed as a percentage of the full detection range.

TABLE 2
Thermal cycle program to measure fluorescence

| Step repeat | Incubation time | Temperature |
|---|---|---|
| 1X | 10 seconds | 25° C. |
| 3X | 10 seconds | 25° C. |
| | 10 seconds | 25° C. |
| | 10 seconds | 60° C. |
| | 10 seconds | 60° C. |
| | 10 seconds | 95° C. |
| | End | |

Results

A serial dilution of molecular weight marker III (III) was made to construct a standard curve. To do this, 20 μl of III was added to 180 μl of nuclease-free water and mixed well. This gave the first standard at a concentration of approximately 25 ng/μl. For each subsequent dilution, 100 μl of the standard was mixed with 100 μl of water. Taking this approach, a two-fold decrease in DNA concentration was achieved. Thirteen DNA standards and one water blank were generated. A 1:10 dilution of a DNA sample of unknown concentration was also made.

The OD₂₆₀ of each standard and the unknown DNA sample were measured using standard spectrophotometry. OD₂₆₀ measurements and the calculated DNA concentration for each sample are given in Table 3. DNA concentration was calculated using the formula:

DNA (μg/μl)=OD₂₆₀×50 μg/μl DNA at 1 OD₂₆₀ absorbance unit

TABLE 3
OD₂₆₀ measurements and DNA concentration of standards and sample

| | OD₂₆₀ less OD₂₆₀ | Calculated DNA |
|---|---|---|
| | of Water | concentration |
| Sample or Standard | Blank | (μg/μl) |
| 1 | 0.424 | 21.2 |
| 2 | 0.185 | 9.25 |
| 3 | 0.068 | 3.4 |
| 4 | 0.011 | 0.55 |
| 5 | −0.02 | — |
| 6 | −0.03 | — |
| 7 | −0.04 | — |
| 8 | −0.04 | — |
| 9 | −0.05 | — |
| 10 | −0.04 | — |
| 11 | −0.04 | — |
| 12 | −0.04 | — |
| 13 | −0.04 | — |
| 1:10 dilution DNA sample | 1.120 | 56 |

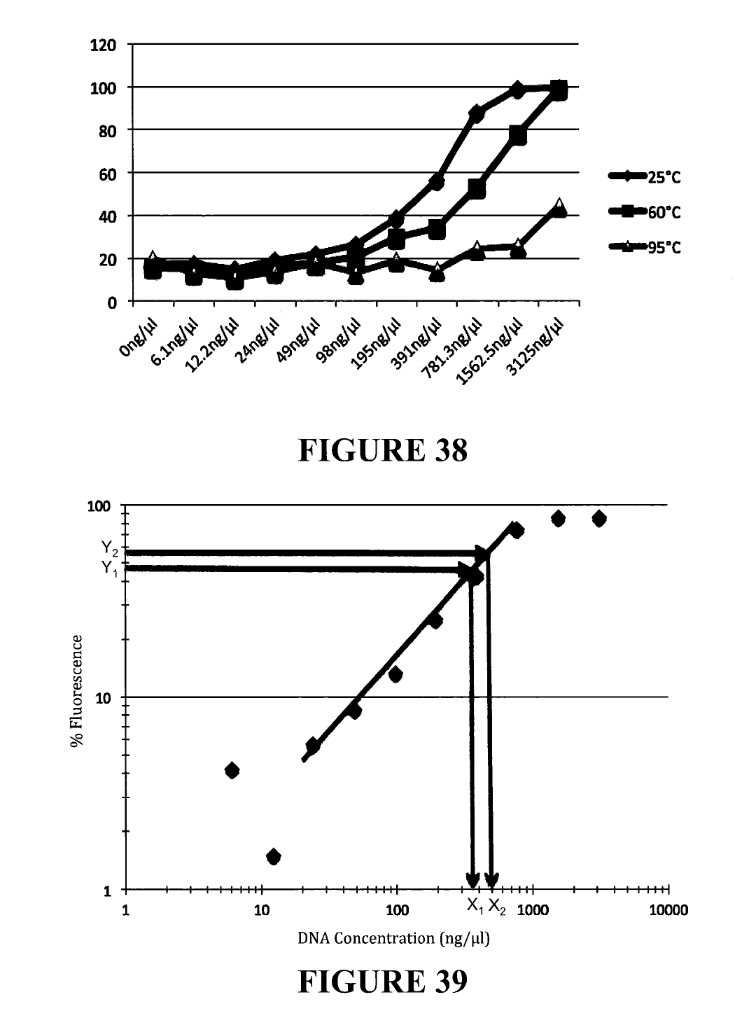

Ten microlitres of either the diluted sample or each standard was added to a custom-made clear acrylic plastic disc chamber (which is similar to the reaction vessel described with reference to FIGS. 1 and 2) and 10 μl of SYBR Green Master Mix I mixed with the sample by pipetting up and down at least ten times. The disc was sealed with sealing foil (Roche LightCycler 480 sealing foils Cat no. 04 729 757 001) ensuring no bubbles were trapped between the film and the solution. The same disc chamber was used for all 1sHHD measurements. Starting with the most dilute standard, the loaded disc chamber was placed on a small droplet of microscope immersion oil (Olympus Optical Co. LTD Immersion Oil 8CC) sitting directly on the diamond thermal plate of the 1sHHD. The oil assisted thermal coupling of the chamber to the diamond thermal plate. Results are given in Table 4 and FIG. 38.

TABLE 4
Average percentage fluorescence of diluted III standard recorded
from the 1sHHD at three different temperatures

| DNA dilution | 25° C. | 60° C. | 95° C. |
|---|---|---|---|
| 0 ng/μl | 17.4 +/− 3.3 | 15.9 +/− 3.1 | 19.7 +/− 2.3 |
| 6.1 ng/μl | 17.5 +/− 1.9 | 15.12 +/− 1.0 | 13 +/− 0.4 |
| 12.2 ng/μl | 14.8 +/− 1.2 | 12.5 +/− 0.6 | 10.7 +/− 0.3 |
| 24 ng/μl | 19 +/− 1.2 | 16.2 +/− 0.7 | 13.6 +/− 0.19 |
| 49 ng/μl | 21.7 +/− 1.7 | 17.8 +/− 0.8 | 17.6 +/− 2.5 |
| 98 ng/μl | 26.6 +/− 3.7 | 20.5 +/− 1.5 | 13.2 +/− 0.5 |
| 195 ng/μl | 38.8 +/− 6.7 | 29.7 +/− 2.7 | 19 +/− 0.3 |
| 391 ng/μl | 56.3 +/− 5.9 | 34.3 +/− 3.3 | 14.7 +/− 0.1 |
| 781.3 ng/μl | 87.9 +/− 10.5 | 53.1 +/− 5.1 | 24.2 +/− 1.2 |
| 1562.5 ng/μl | 99.2 +/− 0.4 | 78.3 +/− 9.7 | 25.7 +/− 3.7 |
| 3125 ng/μl | 99.4 +/− 0.004 | 99.2 +/− 0.2 | 44.5 +/− 3.0 |

FIG. 38 shows the percentage fluorescence from intercollated SYBR Green measured over a serial dilution of Roche III DNA Marker solution taken at three temperatures. Each graphed point is the average of six (temperature 25° C. and 60° C.) and three (95° C.) fluorescence measurements respectively. FIG. 38 graphically demonstrates the effect of temperature on sample fluorescence. As temperature rises DNA progressively denatures. This leads to a decrease in the number of SYBR green molecules intercollated within the DNA molecule and thereby alters the level of fluorescence relative to DNA concentration. The results clearly demonstrate the advantage of a fluorometer with tight thermal control over the nucleic acid sample.

The concentration of an unknown DNA sample was calculated using fluorescence readings taken at 25° C. on the 1sHHD. Fluorescence reading from standards of known concentration were graphed in FIG. 39. Fluorescence readings below reliable detection limits or greater than 99% were excluded from the standard line-of-best-fit curve.

FIG. 39 illustrates how DNA concentration of an unknown sample can be calculated from a standard curve of samples of known DNA concentration using the 1sHHD. The standard curve was constructed with values from Table 4. Fluorescence from two dilutions (1:1600 and 1:1200 respectively) of a sample of unknown concentration are used to demonstrate the determination of the concentration (Y₁ and X₁=48.4% and 370 ng/μl; Y₂ and X₂=55.6% and 500 ng/μl).

Two dilutions of the DNA sample of unknown concentration were measured using the 1sHHD and the corresponding DNA concentrations read from the standard curve as shown in FIG. 39. The dilution factors for the two samples were ×1600 and ×1200 respectively. Multiplying the each standard curve-derived DNA concentration by its respective dilution factor gave similar sample concentrations of 592 μg/μl and 600 μg/μl respectively. This is similar to the concentration of 560 μg/μl measured using spectroscopy.

Conclusion

The 1sHHD successfully measured changes in DNA concentration demonstrating its function as a fluorometer. The precision of specific fluorophore detection was clearly shown by the loss of signal once the DNA was denatured at 95° C. Under these conditions the intercollating dye, SYBR Green, cannot undergo the correct chemical change to emit light of the reporting wavelength. Fluorometry is known to be temperature sensitive as a result of the changing confirmation of DNA at different temperatures. Measurements from the 1sHHD showed this known effect. The usefulness of the 1sHHD to determine DNA concentration of a sample of unknown concentration was also demonstrated. The fluorometric and spectrophotometric results gave reasonably good agreement on DNA concentration. Differences between the OD₂₆₀ reading and 1sHHD are likely to indicate detection of or interference from other molecules in the sample by the spectrophotometer arising from the DNA purification process. Fluorometry based on the SYBR green system only measures the nucleic acid in the sample. These results indicate that the 1sHHD and other devices containing this technology can be used in fluorometry systems.

Experiment 3—Use of Single Channel Hand Held Molecule Detection Device for Detecting Protein

Introduction

A broad range of molecular diagnostic tests are based on the detection of proteins. These tests can use different classes of reagents, for example, antibodies or ligands, to indicate the presence or absence of specific proteins in a sample. For example, a protein test can show presence of troponin in the blood stream indicating heart damage or detection of her-2/neu protein can inform breast cancer treatment. Such tests also can be used to analyse microbial communities. This could be whether a food spoilage organism, like Salmonella, is present as well as the level of microorganism contamination of the item.

A common reporter protein used in molecular diagnostic tests is the Green Fluorescent Protein. This protein was first isolated from Aequorea victoria, a free-swimming Cnidaria that lives off the coast of North America and is the reason for this jellyfish's bioluminescence. The GFP protein has a natural excitation peak of 395 nm and an emission peak of 509 nm. However, since its cooption for use in biological research the fundamental structure of GFP has been genetically engineered to produce a range of GFP family proteins that emit at a plurality of wavelengths ranging from red to blue.

This experiment investigates the application of the preferred embodiments of the device for detecting a commonly used fluorescent reporter protein (GFP) used in molecular diagnostic tests.

Methods

Instrumentation

All measurements were performed using the single channel hand held device (1sHHD) of a preferred embodiment of the invention as described above with reference to FIGS. 7-10. The reaction vessel holder used in the device comprises a synthetic diamond plate.

Test Protein

Biopolymer beads incorporating Green Fluorescent Protein (GFP) were grown in vivo and were supplied as a gift from PolyBactics Ltd (Palmerston North, New Zealand). Biobeads are referred to as GFP protein biobeads. Beads were suspended at 10⁻² dilution in H₂O.

Microscopy

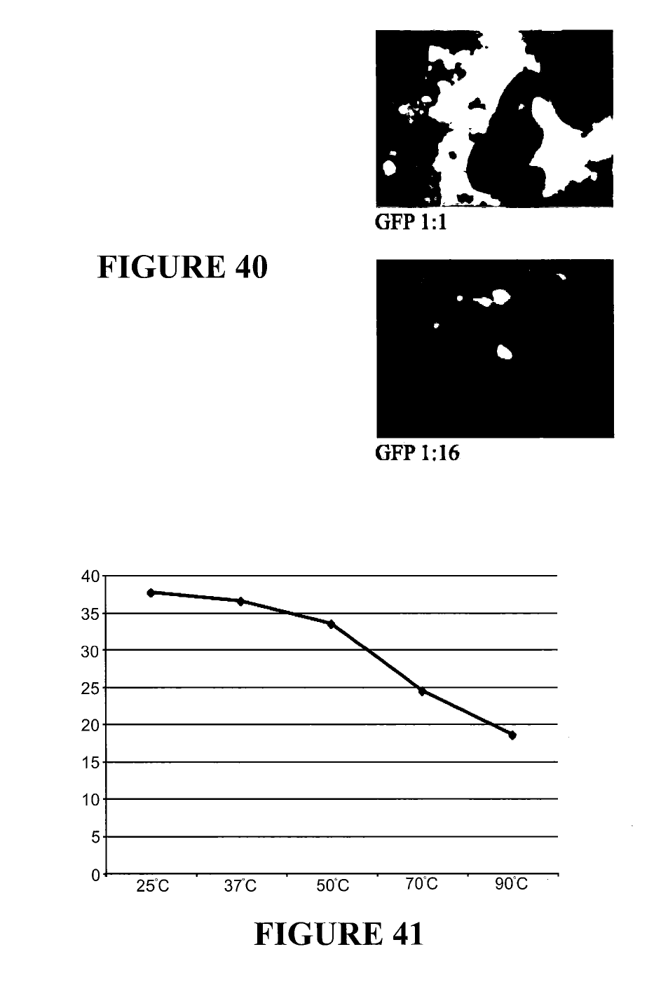

Biobead images were taken using an Olympus AX70 fluorescent microscope. Solution containing the biobeads was spotted onto a microscope slide, coverslipped and viewed at 4× magnification. FIG. 40 shows fluorescence microscopy images of the two dilutions of GFP protein biobeads and the effect of dilution on the level of fluorescence.

Results

A 10⁻² dilution of GFP protein biobeads supplied by PolyBatics Ltd was used on the 1sHHD and fluorescence readings taken at different temperatures. The 1sHHD was programmed to hold the sample at a single temperature and collect 10 fluorescence measurements over a five minute period according to the program given in Table 5. Each sample was measured at five different temperatures: 25° C., 37° C., 50° C., 70° C. and 90° C. Ten microlitres of the sample was place in a clear plastic acrylic reaction chamber and sealed With optically clear adhesive film (AB-Gene, Ref No. AB-0558). The reaction chamber was allowed to equilibrate to temperature for 10 seconds prior to measurement commencing. Average fluorescence readings and standard deviations are given in Table 6 and graphed in FIG. 41. The results show that temperature affected the intensity of GFP fluorescence. This is likely the result of protein confirmation changes at different temperatures. These results point to the potential importance of precise temperature control during measurement.

TABLE 5
1sHHD thermal program for measuring GFP biobead fluorescence

| Step Repeat | Incubation Time | Action at Step |
|---|---|---|
| 1x | 10 seconds | Initial equilabration |
| 10x | 20 seconds | Temperature maintenance |
| | 10 seconds | Record fluorescence |
| | End | |

TABLE 6
Average percentage fluorescence for GFP protein biobeads
at different temperatures

| Temperature | 25° C. | 37° C. | 50° C. | 70° C. | 90° C. |
|---|---|---|---|---|---|
| Average | 37.8 | 36.6 | 33.6 | 24.6 | 18.7 |
| Fluorescence | | | | | |
| Standard | 0.21 | 0.19 | 0.17 | 0.16 | 1.02 |
| deviation | | | | | |

Conclusion

The 1sHHD was able to detect GFP protein. This experiment indicates that temperature has an effect on the strength of the fluorescent signal. These results indicate that the 1sHHD and other devices containing this technology can be used in protein-based reporting and diagnostic systems.

Experiment 4—Optical Transmissivity of the Reaction Vessel Holder

Introduction

The optical performance of a preferred embodiment eighteen channel hand held device was measured.

Methods

Instrumentation
Laser DPSS 473 nm with a 115 mW output
Laser DPSS 532 nm with a 115 mW output
Neutral density (ND) filter optical density (OD) 1.0.
1-into-6 monolithic beam splitter component
6-into-18 monolithic beam splitter component
Powermeter Gigahertz-Optik PT-9610
Diamond Materials Synthetic diamond plate 15×40×0.8 mm

The beam splitter arrangement is an embodiment of the fifth example beam splitter arrangement described with reference to FIG. 16 configured accordingly to output eighteen beams. The primary tiers and second tiers are part of respective monolithic optical components 1151, 1152. Table 7 shows the split configuration of each beam splitter in a tier.

TABLE 7
Optical configuration of the 1-into-6 monolithic component 1151
and 6-into-18 monolithic component 1152

| | Beam intensity (~) | | |
|---|---|---|---|
| | Toward the | | Toward the |
| Tier | next tier | Toward tier 6 | reaction vessel |
| 1-into-6 monolithic | | | |
| component 1151 | | | |
| 1 | 83% | 17% | — |
| 2 | 80% | 20% | — |
| 3 | 75% | 25% | — |
| 4 | 67% | 33% | — |
| 5 | 50% | 50% | — |
| 6-into-18 monolithic | | | |
| component 1152 | | | |
| 6 | 67% | — | 33% |
| 7 | 50% (toward the | — | 50% |
| | reaction vessel) | | |

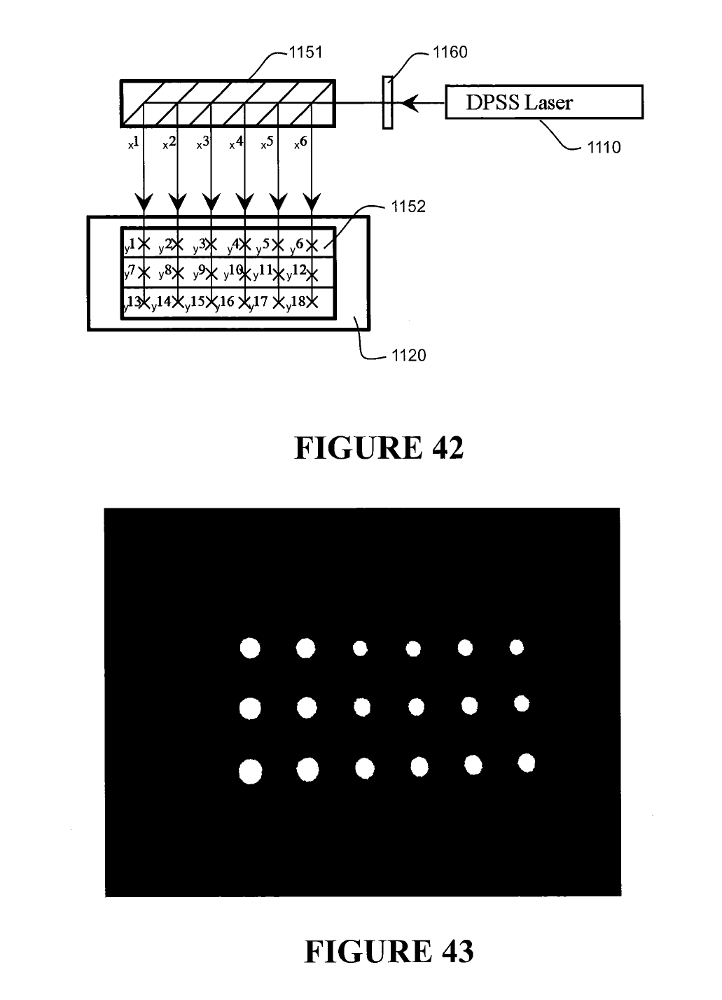

Referring to FIG. 42, the output from the laser 1110 was directed through the ND filter 1160, which attenuates the beam by a factor of ten. The attenuated beam was then directed to the 1-into-6 monolithic beam splitter component 1151. Each beam splitter of the 1-into-6 monolithic beam splitter component 1151 is configured to split an incoming beam according to the split ratios defined in Table 7. The six output beams from the 1-into-6 monolithic beam splitter component 1151 was then directed to the 6-into-18 monolithic beam splitter component 1152. The ‘O’ locates the eighteen beams (y1 to y18) as emanating from the page towards the viewer. Each beam splitter of the 6-into-18 monolithic beam splitter component 1152 is configured to split an incoming beam according to the split ratio defined in Table 7. The eighteen beams then pass through the synthetic diamond plate 1120, positioned above the 6-into-18 monolithic component 1152. Laser power measurements were taken at the laser output coupler, after the ND filter 1110, at the output from the 1-into-6 monolithic component 1151, at the output of the 6-into-18 monolithic component 1152, and after the diamond plate 1120 respectively.

The optical transmissivity of the diamond plate 1120 was first tested using the 532 nm laser, followed by the 473 nm laser.

Results

Using 532 nm System Laser

Table 8 shows how the laser energy incident on the 1-into-6 monolithic beam splitter component 1151 was partitioned in channels x1-x6 (depicted in FIG. 42).

TABLE 8
Power results of the split excitation beams from the 1-into-6
monolithic component 1151 for the 532 nm laser at 14.35 mW

| | Channel | | | | | | |
|---|---|---|---|---|---|---|---|
| | x1 | x2 | x3 | x4 | x5 | x6 | Total |
| Power mW | 2.59 | 2.31 | 1.47 | 1.36 | 1.48 | 1.59 | 10.80 |

Beam splitter efficiency was defined as the power incident on the beam splitter divided by the sum of the power over the output channels. The efficiency of the 1-into-6 monolithic component 1151 under 532 nm illumination was 10.8/14.35=0.7526 or approximately 75%

Table 9 shows how the laser channels from the 1-into-6 monolithic component 1151 were partitioned by the 6-into-18 monolithic component 1152 in channels y1-y18 (depicted in FIG. 42). Column ‘B’ is the power reading before the diamond plate 1120 and column ‘A’ is after the diamond plate 1120.

TABLE 9
Power results of the split excitation beams from the 6-into-18
monolithic component 1152 for the 532 nm laser at 14.35 mW

| | B | A | | B | A | | B | A | Total B | Total A | Trans |
|---|---|---|---|---|---|---|---|---|---|---|---|
| CH | mW | mW | CH | mW | mW | CH | mW | mW | mW | mW | % |
| y1 | 0.57 | 0.35 | y7 | 0.63 | 0.41 | y13 | 0.64 | 0.40 | 1.84 | 1.16 | 63.0 |
| y2 | 0.51 | 0.29 | y8 | 0.58 | 0.39 | y14 | 0.58 | 0.36 | 1.67 | 1.04 | 62.3 |
| y3 | 0.35 | 0.20 | y9 | 0.35 | 0.23 | y15 | 0.36 | 0.23 | 1.06 | 0.66 | 62.3 |
| y4 | 0.26 | 0.18 | y10 | 0.35 | 0.23 | y16 | 0.35 | 0.21 | 0.96 | 0.62 | 64.6 |
| y5 | 0.28 | 0.19 | y11 | 0.38 | 0.24 | y17 | 0.34 | 0.21 | 1.00 | 0.64 | 64.0 |
| y6 | 0.33 | 0.20 | y12 | 0.40 | 0.24 | y18 | 0.41 | 0.23 | 1.14 | 0.67 | 58.8 |
| Total | 2.30 | 1.41 | | 2.69 | 1.74 | | 2.68 | 1.64 | 7.67 | 4.79 | 62.5 |
| | | | | | | | | | | | ave |

The efficiency of the 6-into-18 monolithic component 1152 under 532 nm illumination was 7.67/10.8=0.71 or 71%. Efficiency of the overall beam splitter arrangement was 0.7526×0.71=0.5343 or approximately 53%.

The total system efficiency which includes losses in the diamond plate 1160 was 4.79/14.35=0.3338 or approximately 33%.

Using 473 nm System Laser

Table 10 shows how the laser energy incident on the primary tiers of the beam splitter arrangement was partitioned in channels x1-x6 (depicted in FIG. 42). The ND filter 1160 of OD1 was omitted for this experiment.

TABLE 10
Power results of the split excitation beams from the 1-into-6
monolithic component 1151 for the 473 nm laser at 100 mW.

| | Channel | | | | | | |
|---|---|---|---|---|---|---|---|
| | x1 | x2 | x3 | x4 | x5 | x6 | Total |
| Power mW | 17.0 | 14.80 | 9.1 | 9.4 | 10.0 | 9.6 | 69.9 |

The efficiency of the 1-into-6 monolithic component 1151 under 473 nm illumination was therefore 69.9/100.0=0.699 or approximately 70%.

Table 11 shows how the laser channels from the primary tiers were partitioned by the 6-into-18 monolithic component 1152 in channels y1-y18 (depicted in FIG. 42). Column ‘B’ is the power reading before the diamond plate 1120 and column ‘A’ is after the diamond plate 1120.

TABLE 11
Power results of the split excitation beams from the 6-into-18
monolithic component 1152 for the 473 nm laser at 100 mW

| | B | A | | B | A | | B | A | Total B | Total A | Trans |
|---|---|---|---|---|---|---|---|---|---|---|---|
| CH | mW | mW | CH | mW | mW | CH | mW | mW | mW | mW | % |
| y1 | 3.9 | 2.3 | y7 | 4.10 | 2.30 | y13 | 4.9 | 3.00 | 12.90 | 7.60 | 58.9 |
| y2 | 3.3 | 2.0 | y8 | 3.45 | 2.20 | y14 | 4.2 | 2.62 | 10.95 | 6.82 | 62.3 |
| y3 | 2.1 | 1.35 | y9 | 2.20 | 1.50 | y15 | 2.7 | 1.75 | 7.00 | 4.60 | 65.7 |
| y4 | 2.13 | 1.40 | y10 | 2.20 | 1.50 | y16 | 2.7 | 1.75 | 7.03 | 4.65 | 66.1 |
| y5 | 2.25 | 1.47 | y11 | 2.40 | 1.63 | y17 | 2.8 | 1.85 | 7.45 | 4.95 | 66.4 |
| y6 | 2.15 | 1.41 | y12 | 2.30 | 1.50 | y18 | 2.8 | 1.81 | 7.25 | 4.72 | 65.1 |
| Total | 15.83 | 9.93 | | 16.65 | 10.63 | | 20.1 | 12.78 | 52.58 | 33.34 | 64.1 |
| | | | | | | | | | | | ave |

The efficiency of the 6-into-18 monolithic component 1152 under 473 nm illumination was 52.58/69.90=0.752 or 75%. Efficiency of the overall beam splitter arrangement was 0.752×0.0.699=0.5258 or approximately 53%.

The total system efficiency which includes losses in the diamond plate 1120 was 33.34/100=0.3334 or approximately 33%.

FIG. 43 shows the output of the eighteen split excitation beams after the diamond plate 1120 on a white sheet of paper. Each of these beams will be directed into a respective reaction vessel chamber containing a sample for analysis.

Conclusions

The results show a variation of up to a factor of two in laser power partitioning per channel for both the 1-into-6 monolithic component 1151 and the 6-into-18 monolithic component 1152 under 532 nm and 473 nm laser operation. Individual beam splitter component efficiency was about 70-75%, due partially to the metallic coatings used in the beam splitters. This result was expected and of no significance for the 18-channel device as abundant laser power is available.

The variation is channel power is also due to the tolerances available for the reflectance of the metallic coatings and of no significance for the 18-channel device. Each of the 18 channels will require trimming of the optical power via a dedicated ND filter after the 6-into-18 channel monolithic beam splitter.

There were no observable wavelength dependent absorption effects when switching from the 473 nm laser to the 532 nm laser. If present, these effects were insignificant to the operation of the 18-channel device. Partitioning of laser energy does not appear to be a function of system laser power. The beam splitter efficiency was similar for both 532 nm and 473 nm system laser operation.

Transmission of the diamond plate 1120 was similar to 62.5% mean for 532 nm illumination and similar to 64.1% mean for 473 nm illumination. Some variation in transmission efficiency may be due to variation in the surface polish figures over the extent of the diamond plate 1120 in addition to local variation in impurity density which while small in optical grade synthetic diamond is nevertheless still present.

Experiment 5—Use of the Reaction Vessel Holder to Amplify by PCR DNA Targets in Multiple Samples Simultaneously

Introduction

The performance of the reaction vessel for amplifying DNA in multiple samples simultaneously was measured against a standard laboratory-based end-point PCR instrument supplied commercially from Applied Biosystems. This commercial system was the GeneAmp 9700 (GA).

The PCR assay used for this work amplified the Jellyfish Green Fluorescent Protein (GFP) sequence encoded in cloning vector eGFP-N1 from CLONTECH. PCR reactions were run in parallel on both the reaction vessel holder thermally cycled by the 1sHHD and the GA instruments. Comparison between the technologies was facilitated through agarose gel electrophoresis. All reactions shared the same PCR assay components and used the same sealing foil and thermal-cycling conditions. Each reaction was set up from the same master mix and was carried out on both instruments at the same time.

Methods

Instrumentation:

Two devices were used in the tests outlined below. These were the GeneAmp 9700 PCR machine (GA) from Applied Biosystems and the single sample hand held device (1sHHD) as described above with reference to FIGS. 7-10. The reaction vessel holder used in the 1sHHD device comprises a synthetic diamond plate.

Source of PCR Template:

Genomic DNA (gDNA) from a GFP transgenic mouse was used to generate template for this work. The gDNA was extracted from 22 mg of GFP transgenic mouse liver using the ZyGem prepGEM Tissue kit as per the manufacturer's instructions. This gave a transgenic gDNA sample with a concentration of 20 ng/μl.

The CLONTECH eGFP-N1 vector was used to construct the GFP transgenic mouse. PCR primers were designed to this vector to amplify the GFP DNA encoding sequence. The NCBI primer design tool, available on the NCBI website (http://ncbi.nlm.nih.gov) was used to design the forward and reverse primers for GFP. Primers are given in Table 12.

TABLE 12
PCR Primers

| Primer Name | Sequence |
|---|---|
| eGFP-Short | TTCAGCCGCTACCCCGACCA (SEQ ID NO: 1) |
| Forward | |
| eGFP-Short | CGGTTCACCAGGGTGTCGCC (SEQ ID NO: 2) |
| Reverse | |

The 20 μl PCR reaction used to make template for subsequent experiments contained the following mix of components: 10 μl SYBR Green I Master Mix (Roche, Cat No. 04 707 516001); 1 μl (20 pmol) of both Forward and Reverse Primer; 7 μl PCR quality H₂O; and 1 μl of a 10⁻⁶ dilution of the transgenic gDNA sample. The GFP amplicon was generated by PCR on the GeneAmp 9700 (Applied Biosystems) instrument using thermal cycle conditions 95° C. for five minutes followed by 40 cycles of 95° C. for 30 seconds, 60° C. for 30 seconds and 72° C. for 30 seconds. These three temperatures constitute a cycle: DNA denaturation at 95° C., primer annealing at 60° C. and primer extension at 72° C. The amplicon was checked by agarose gel electrophoresis (data not shown) to confirm production of the correct sized product. This amplicon was diluted by 10⁻⁴ to give the Initial DNA Template for the experiments described here.

PCR Method:

Each PCR reaction mix contained 5 μl SYBR Green I Master Mix (Roche, Cat No. 04 707 516001); 1 μl (20 pmol) of both Forward and Reverse Primer; 2 μl PCR quality H₂O; and 1 μl of DNA or H₂O. A Master Mix of PCR reagents consisting of enough reagents to perform multiple reactions was set up for each experiment. To do this, the volumes required for each component making up a 10 μl reaction were multiplied by the number of reactions needed for the experiment (in this case 6) plus one extra to allow for pipetting errors (7).

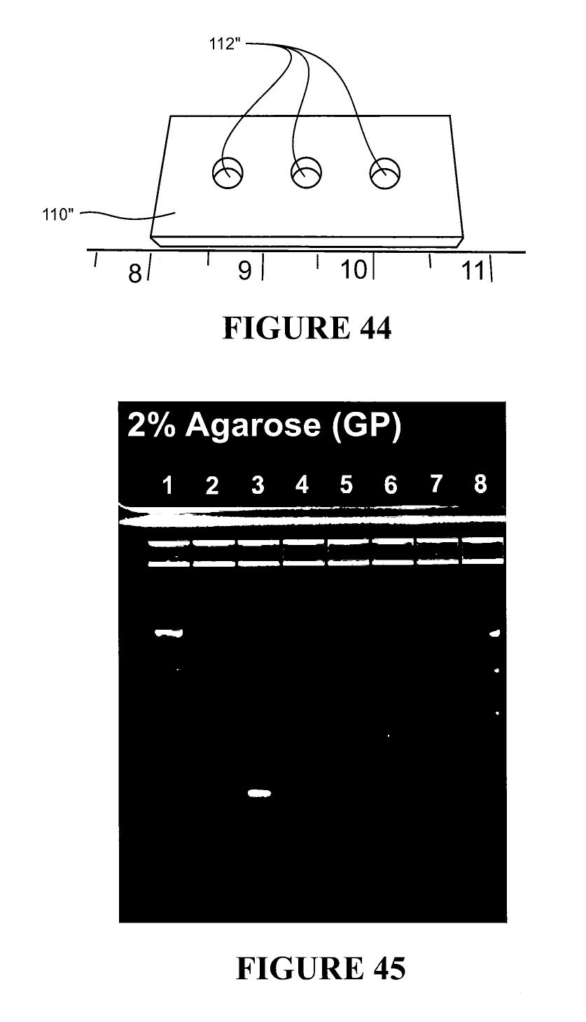

PCR reactions carried out using the GA used 200 μl plasticware domed tubes. Referring to FIG. 44, PCR reactions performed on the 1sHHD were undertaken in a custom-made white plastic plate 110″ with three independent chambers 112″, which was sealed with Roche sealing foil (Cat No. 04 729 757 001).

Identical thermal-cycle conditions were used on both devices to amplify the target GFP sequence from template. These conditions were 95° C. for 3 minutes followed by 40 cycles of 95° C. for 20 seconds, 60° C. for 20 seconds and 72° C. for 20 seconds. These three temperatures constitute a cycle: DNA denaturation at 95° C., primer annealing at 60° C. and primer extension at 72° C.

Results

Sufficient master mix for six reactions was prepared without the addition of DNA template. Ten microlitres of this mix was removed as a ‘no template’ control to be run on the GA. DNA template for five reactions was then added to the remaining master mix and the contents vortexed. An additional ten microlitres of the reaction mix was removed from the master mix and stored on ice. This was the ‘before amplification’ control for all reactions that had DNA added to them. Ten microlitre aliquots of the remaining master mix were added to the three chambers 112″ in the custom plastic plate 110″ for use with the 1sHHD and to a single 200 μl domed tube for amplification on the GA.

The ‘no template control’ and the reaction in the 200 μl tube were subjected to 40 cycles of amplification using the GA. The samples transferred to the custom plastic plate 110″ were placed onto the synthetic diamond plate of the 1sHHD. A drop of Immersion Oil (Olympus Optical Co. LTD Immersion Oil 8CC) was added between the custom plastic plate 110″ and the synthetic diamond plate to facilitate heat transfer to the PCR reaction. A weighted styrofoam insulator pad was placed on top of the custom plastic plate 110″ to prevent excessive heat loss from the top of the plate 110″. The 1sHHD was run in the open configuration and a box was placed over the instrument to reduce ambient air movement around the device during operation. The custom plastic plate 110″ was then subjected to 40 cycles of PCR amplification.

At the completion of thermal cycling, 5 μl aliquots of each reaction were subjected to end-point PCR analysis by electrophoresis on an agarose gel (E-Gel 2% Agarose GP, Life Sciences Cat No. G501802). The results are shown in FIG. 45. The 8 lanes show 1) 2 μl XIV Roche DNA size standard (Cat No. 11 721 933 001); 2) ‘no template control’ after amplification on the GA; 3) DNA amplicons after amplification on the GA; 4) Reaction mix with DNA before amplification; 5) Reaction mix with DNA after amplification on the synthetic diamond plate in the 1sHHD, in a first one of the chambers 112″ of the custom plastic plate 110″; 6) Reaction mix with DNA after amplification on the synthetic diamond plate in the 1sHHD, in a second one of the chambers 112″ of the custom plastic plate 110″; 7) Reaction mix with DNA after amplification on the synthetic diamond plate in the 1sHHD, in a third one of the chambers 112″ of the custom plastic plate 110″; 8) 2 μl XIV Roche DNA size standard.

FIG. 45 shows that DNA fragments of the expected sizes were amplified in all three independent chambers 112″ of the custom plastic plate 110″ at the same time using the same synthetic diamond plate in the same run. The ‘no template control’ showed the presence of background levels of DNA but this material did not correspond to the expected DNA fragment sizes shown in the template control in lane 3. Lane 4 clearly shows that, without heat transfer to the sample for the PCR amplification process, DNA could not be detected in the reaction mix by agarose gel electrophoresis. This shows that detection of DNA could only occur through the successful application of the PCR process.

Conclusion

The results show the successful amplification of multiple samples on a single reaction vessel holder (the synthetic diamond plate).

Summary of Sequences

| SEQ ID NO | Sequence | Type | Reference |
|---|---|---|---|
| 1 | Polynucleotide | Artificial, primer | eGFP-Short Forward |
| 2 | Polynucleotide | Artificial, primer | eGFP-Short Reverse |

The above describes a preferred embodiment(s) of the invention. Modifications and improvements may be made without departing from the scope of the invention.

It is not the intention to limit the scope of the invention to the abovementioned examples only.

For example, any of the described and shown embodiments may have one or more features of other embodiments.

Other modifications include those described in the Summary of the Invention section.

1-49. (canceled)

50. A device for molecule analysis of a sample in a reaction vessel, the device comprising:
a reaction vessel holder for receiving the reaction vessel, the reaction vessel for containing the sample and having at least one portion that is substantially optically transparent to light of at least a first range of wavelengths, the reaction vessel holder comprising a body arranged to thermally couple to and support the reaction vessel,
a thermal device, thermally coupled to the body, that heats and/or cools the reaction vessel holder and thereby the reaction vessel,
an excitation arrangement that generates one or more excitation beams to stimulate an emission of reaction light from the sample; and
a detector arrangement that detects the reaction light from the sample,
wherein the body comprises at least one transparent portion that is substantially optically transparent to the light of the at least a first range of wavelengths, such that the optically transparent portion of the reaction vessel is adapted to face the transparent portion of the body such that light of the first range of wavelengths to and/or from the sample in the reaction vessel pass through the transparent portion of the reaction vessel,
and wherein the excitation arrangement and the detector arrangement are positioned at or facing opposite sides of the reaction vessel such that an optical path of the excitation arrangement and an optical path of the detector arrangement are substantially collinear or parallel.

51. The device of claim 50, wherein the excitation arrangement and the detector arrangement are positioned on or facing opposite sides of the transparent portion of the body.

52. The device of claim 51, wherein the positioning of the excitation arrangement and the detector arrangement is such that a reaction vessel received by the reaction vessel holder is sandwiched between the excitation arrangement and the detector arrangement.

53. The device of claim 50, wherein a distance between the reaction vessel holder and the detector arrangement is up to 20 mm.

54. The device of claim 53, wherein the distance is between 10 mm and 20 mm.

55. The device of claim 54, wherein the distance is between 10 mm and 15 mm.

56. The device of claim 50, wherein the body has a thermal conductivity of about 25 Wm⁻¹K⁻¹ or higher.

57. The device of claim 56, wherein the body has a thermal conductivity of between about 1800 Wm⁻¹K⁻¹ and about 2100 Wm⁻¹K⁻¹.

58. The device of claim 56, comprising a thermal device thermally coupled to the body and positioned on the body laterally to the optical path.

59. The device of claim 58, wherein the thermal device is configured for thermoelectric cooling and heating employing the Peltier effect.

60. The device of claim 59, wherein the thermal device is a single thermoelectric cooling unit and the body is configured to thermally cycle at up to about 20° C. per second, or wherein the thermal device comprises two thermoelectric cooling units and the body is configured to thermally cycle at up to about 40° C. per second.

61. The device of claim 58, wherein the thermal device comprises two thermoelectric cooling units, and wherein the reaction vessel holder is physically mounted to each of the thermoelectric cooling units.

62. The device of claim 58, further comprising a temperature sensor that senses the temperature of the reaction vessel holder, wherein the temperature sensor is in electronic communication with a controller that controls an operation of the thermal device, wherein the thermal device heats or cools the reaction vessel holder.

63. The device of claim 50, wherein the reaction vessel holder is arranged to receive a plurality of reaction vessels, and wherein the reaction vessel holder has respective substantially optically transparent portions corresponding to the reaction vessels.

64. The device of claim 63, comprising a beam splitter arrangement for splitting one or more excitation beams from the excitation arrangement, wherein respective beams of said one or more excitation beams correspond with respective substantially optically transparent portions.

65. The device of claim 64, wherein the beam splitter arrangement provides for split excitation beams that are substantially parallel and/or substantially orthogonal to each other.

66. The device of claim 64, wherein the beam splitter arrangement comprises a beam steering device.

67. The device of claim 50, wherein the reaction vessel holder is arranged to physically mount to the thermal device.

68. The device of claim 50, comprising a reaction vessel securing member that removably secures the reaction vessel to the reaction vessel holder.

69. A method for detection of one or more molecules in a sample contained in a reaction vessel, comprising:
providing a reaction vessel having a portion that is substantially optically transparent to a light of at least a first range of wavelengths,
thermally coupling the reaction vessel to a reaction vessel holder comprising a body arranged to thermally couple to and support the reaction vessel, the body further comprising at least one transparent portion that is substantially optically transparent to the light of the at least a first range of wavelengths, such that the optically transparent portion of the reaction vessel is adapted to face the transparent portion of the body such that light of the first range of wavelengths to and/or from the sample in the reaction vessel pass through the transparent portion of the reaction vessel; and
guiding light to and/or from the sample in the reaction vessel through the transparent portion of the reaction vessel holder and through the transparent portion of the reaction vessel.
